(19) United States
(12) Patent Application Publication
(10) Pub. No.: US 2018/0371455 A1
(43) Pub. Date: Dec. 27, 2018

(54) Arrays of Intersecting Double Stranded Nucleic Acid Helices

(71) Applicants: Emory University, Atlanta, GA (US); Purdue University, West Lafayette, IN (US)

(72) Inventors: Yonggang Ke, Sandy Spring, GA (US); Travis Meyer, Atlanta, GA (US); Pengfei Wang, Decatur, GA (US); Jie Song, Atlanta, GA (US); Chengde Mao, West Lafayette, IN (US); Zhe Li, West Lafayette, IN (US)

(21) Appl. No.: 16/014,455

(22) Filed: Jun. 21, 2018

Related U.S. Application Data
(60) Provisional application No. 62/523,145, filed on Jun. 21, 2017.

(51) Int. Cl. C12N 15/10

(52) U.S. Cl. CPC C12N 15/1093

(57) ABSTRACT

This disclosure relates to intersecting double stranded nucleic acid helices with flexible intersections that form antijuction units. In a typical embodiment, the configuration of the intersecting double stranded nucleic acid helices can be altered by hybridization with an added single stranded segment or trigger strand reconfiguring the conformation of the array in a directional manor. In certain embodiments, the dynamic antijunction unit contains four nucleic double-helix domains, typically of approximately equal length and four dynamic nicking or intersecting flex points providing an array with the capability of switching between two or more stable conformations, e.g., through an intermediate open conformation.

### CROSS-REFERENCE TO RELATED APPLICATIONS

This application claims the benefit of U.S. Provisional Application No. 62/523,145 filed Jun. 21, 2017. The entirety of this application is hereby incorporated by reference for all purposes.

### STATEMENT REGARDING FEDERALLY SPONSORED RESEARCH OR DEVELOPMENT

This invention was made with government support under grants DMR-1654485 and CMMI-1437301 awarded by the National Science Foundation, and under grant N00014-15-1-2707 awarded by the Office of Naval Research. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 17176US_ST25.txt. The text file is 98 KB, was created on Jun. 21, 2018, and is being submitted electronically via EFS-web.

### BACKGROUND

Information relay at the molecular level is an essential phenomenon in numerous chemical and biological processes. A key challenge in synthetic molecular self-assembly is to construct artificial structures that imitate these complex dynamic behaviors in controllable systems. Thus, there is a need to identify synthetic structures that replicate complex dynamic behaviors.

The basic principle of DNA nanostructure design is to engineer structural information into the DNA sequences by programming complementarity between component DNA strands. Approaches for constructing DNA nanostructures largely fall into two major categories: DNA origami and DNA tiles. DNA origami is a “folding” method, in which a long “scaffold” strand (often M13 viral genomic DNA) is folded into a prescribed shape via interactions with hundreds of short, synthetic “staple” strands.

The DNA tile method assembles DNA structures by connecting small structural units, typically consisting of a small number of strands. The DNA origami approach has produced fully addressable structures up to several thousands of base pairs (bp). Similar-sized, fully addressable structures have also been fabricated with special types of DNA tiles, such as single-stranded tiles or DNA bricks.

Ke et al. report three-dimensional structures self-assembled from DNA bricks. Science, 2012, 338, 1177-1183. Jiang et al. report programmable DNA hydrogels assembled from multidomain DNA strands, Chembiochem, 2016, 17(12):1156-62. See also Ke, Designer three-dimensional DNA architectures, Curr Opin Struct Biol, 2014, 27:122-8, WO 2014/018675, and WO 2015/138231.

Feng et al. report a two-state DNA lattice switched by DNA nanoactuator. Angew Chem Int Ed Engl, 2003, 42(36):4342-6.

References cited herein are not an admission of prior art.

### SUMMARY

This disclosure relates to intersecting double stranded nucleic acid helices with flexible intersections that form dynamic antijunction units. In a typical embodiment, the configuration of the intersecting double stranded nucleic acid helices can be altered by hybridization with an added single stranded segment or trigger strand reconfiguring the conformation of the array in a directional manor. In certain embodiments, the dynamic antijunction unit contains four nucleic double-helix domains, typically of approximately equal length and four dynamic nicking or intersecting flex points providing an array with the capability of switching between two or more stable conformations, e.g., through an intermediate open conformation.

In certain embodiments, this disclosure contemplates method of initiating a transformation cascade with preassembled arrays. In certain embodiments, the transformation is initiated starting from one conformation by the addition of a trigger strand at selected locations of the array. Structural transformation from the selected sites propagates to the rest of the array in a stepwise manner, typically without additional trigger strands at other locations. Releasing the old trigger strands and adding new triggers can transform the array back to its initial conformation resulting in a reversible process. In certain embodiments, the triggering methods disclosed herein, which results in altered configurations, can be repeated for multiple rounds.

An object of this disclosure is to provide nucleic acid arrays wherein the propagation pathway follows prescribed routes, as well as to stop and then resume propagation by mechanically decoupling the antijunctions or introducing “block strands.” In certain embodiments, the kinetics of array transformation can be enhanced by elevated temperatures or adding an agent the interrupts hydrogen bonding, e.g., formamide. In certain embodiments, assembly and transformations are evaluated by atomic force microscopy and agarose gel electrophoresis.

In certain embodiments, the nucleic acid arrays disclosed herein can be used in methods for controlled, multistep, long-range transformation, assembled by interconnected modular dynamic units that can transfer their structural information to neighboring units. The dynamic behavior of the nucleic acid array can be regulated by external factors such as the shapes and sizes of the arrays, the initiation of transformation at selected units, and the engineered information propagation pathways. In certain embodiments, this disclosure relates to uses of the nucleic acid array construction in devices to detect, translate molecular interactions to conformational changes in nucleic acid structures, and trigger remotely subsequent molecular events.

In certain embodiments, this disclosure relates to double helix arrays of nucleic acids comprising intersections of four double stranded arms, wherein a first double stranded arm and a second double stranded arm share a first polynucleotide strand, wherein the second double stranded arm and a third double stranded arm share a second polynucleotide strand, wherein the third double stranded arm and a fourth double stranded arm share a third polynucleotide strand, and wherein the fourth double stranded arm and the first double strand arm share a fourth polynucleotide strand; and wherein the first double stranded arm and the second double stranded arm form a first ring, and the third double stranded arm and the fourth double stranded arm form a second ring. In certain embodiments, the double helix array disclosed herein comprises two, three, four, five, six, seven, eight, nine, ten, fifteen or more intersections.

In certain embodiments, the second ring comprises a segment that is single stranded. In certain embodiments, the second ring is located on the boundary of the array.

In certain embodiments, the second double stranded arm and the third double stranded arm form a third ring, and the first double stranded arm and the fourth double stranded arm form a fourth ring. In certain embodiments, the double helix array disclosed herein comprises five, six, seven, eight, nine, ten, fifteen or more rings.

In certain embodiments, the first polynucleotide strand substantially hybridizes with the array to form a double helix, and optionally comprises a terminal 5′ single stranded polynucleotide segment and/or a terminal 3′ single stranded polynucleotide segment that does not hybridize with the array.

In certain embodiments, the second polynucleotide strand substantially hybridizes with the array to form a double helix, and optionally comprises a terminal 5′ single stranded polynucleotide segment and/or a terminal 3′ single stranded polynucleotide segment that does not hybridize with the array.

In certain embodiments, the third polynucleotide strand substantially hybridizes with the array to form a double helix, and optionally comprises a terminal 5′ single stranded polynucleotide segment and/or a terminal 3′ single stranded polynucleotide segment that does not hybridize with the array.

In certain embodiments, the fourth polynucleotide strand substantially hybridizes with the array to form a double helix, and optionally comprises a terminal 5′ single stranded polynucleotide segment and/or a terminal 3′ single stranded polynucleotide segment that does not hybridize with the array.

In certain embodiments, the first polynucleotide strand substantially hybridizes with the array to form a double helix, and comprises a terminal 5′ single stranded polynucleotide segment that does not hybridize with the array and a terminal 3′ single stranded polynucleotide segment that does not hybridize with the array, and the array further comprises a fifth polynucleotide segment wherein the fifth polynucleotide segment that hybridizes with the terminal 5′ single stranded polynucleotide segment of the first polynucleotide strand and hybridizes with the terminal 3′ single stranded polynucleotide segment of the first polynucleotide strand.

In certain embodiments, the first polynucleotide strand substantially hybridizes with the array to form a double helix, and optionally comprises a terminal 5′ single stranded polynucleotide segment and/or a terminal 3′ single stranded polynucleotide segment that does not hybridize with the array, and further comprises a fifth polynucleotide segment wherein the fifth polynucleotide segment hybridizes with the terminal 5′ single stranded polynucleotide segment of the first polynucleotide strand or hybridizes with the terminal 3′ single stranded polynucleotide segment of the first polynucleotide strand and the fifth polynucleotide segment hybridizes with the terminal 5′ single stranded polynucleotide segment of the second polynucleotide strand or hybridizes with the terminal 3′ single stranded polynucleotide segment of the second polynucleotide strand.

In certain embodiments, a boundary of the array comprises terminal 5′ single stranded polynucleotide sequence and/or a terminal 3′ single stranded polynucleotide sequence comprising segments of poly-T.

In certain embodiments, this disclosure relates to aqueous solutions comprising an intersecting double helix array as disclosed herein and optionally formamide. In certain embodiments, the aqueous solutions are heated to above 40 degrees Celsius.

In certain embodiments, the array is configured into a tube structure or other three-dimensional structure, or other curved structure.

In certain embodiments, the array is linked through covalent bonding or hydrogen bonding or other non-covalent bonding to a ligand, receptor, drug, antibody, aptamer, other specific binding agent, or combinations thereof.

In certain embodiments, the array is linked through covalent or hydrogen bonding to a solid surface such as glass, plastic, metal, or particle. In certain embodiments, a plurality of arrays are separated into a plurality of particles, zones or wells, e.g., such that only one array is in each zone or well.

In certain embodiments, this disclosure relates to method of transforming the confirmation of a double helix array comprising mixing the double helix array as disclosed herein and an added single stranded nucleic acid which is configure to hybridize with the segment that is single stranded on the second ring under conditions such that the added single stranded nucleic acid bends or straightens the segment that is single stranded altering the conformation of the double helix array.

In certain embodiments, the added single stranded nucleic acid is conjugated to a detectable moiety, fluorescent moiety, ligand, receptor, drug, enzyme, antibody, aptamer, or other antigen binding moiety.

### BRIEF DESCRIPTION OF THE DRAWING

FIG. 1A illustrates a double helix array of nucleic acids comprising an intersection of four double stranded arms at a flex point (1), wherein a first double stranded arm (2) and a second double stranded arm (3) share a first polynucleotide sequence (4), wherein the second double stranded arm (3) and a third double stranded arm (5) share a second polynucleotide sequence (6), wherein the third double stranded arm (5) and a fourth double stranded arm (7) share a polynucleotide sequence (8), and wherein the fourth double stranded arm (7) and the first double strand arm (2) share a fourth polynucleotide sequence (9); and wherein the first double stranded arm (2) and the second double stranded arm (3) form a first ring (10), and the third double stranded arm (5) and the fourth double stranded arm (7) form a second ring (11). The second ring (11) comprises a segment that is single stranded (12). The second double stranded arm (3) and the third double stranded arm (5) form a third ring (13), and the first double stranded arm (2) and the fourth double stranded arm (7) form a fourth ring (14). The arrows illustrate individual polynucleotides. The light gray and dark arrows are configured to hybridize with each other. As referred to herein, “sharing” a polynucleotide refers to hybridizing such that a double helix having the polynucleotide is formed on both of the arms as illustrated in the bends and corners shown in FIG. 2A.

FIG. 1B illustrates anti junctions—an antijunction unit consists of four duplexes of same length. Each arm is 0.5×n turns (n=1, 2, 3, 4 . . . ).

FIG. 2A illustrates a dynamic DNA antijunction unit can switch between two stable conformations, through an unstable open conformation.

FIG. 2B shows different diagrams for a DNA antijunction units: stable conformations “1st” and “2nd,” and unstable conformation “intermediate.”

FIG. 2C illustrates transformation of an antijunction unit can be induced by addition of a trigger strand. The information is passed from the converted unit to its closest neighbors, causing them to undergo subsequent transformation. A trigger strand removes a mobile nick point from anti junction unit.

FIG. 2D shows a strand diagram of an interconnected 2D DNA relay array. Three trigger strands are added to three units in the upper left corner of the array to initiate the transformation.

FIG. 2E illustrates the information of transformation propagates along prescribed pathways, causing the units to convert sequentially in this molecular array.

FIG. 2F illustrates the design of arrays. A 6 units by 4 units 32-bp relay array containing 24 32-bp antijunctions. On the right, the strand extensions at the edges represents poly-T sequences. Left strand diagram of a 6 units by 4 units 32-bp relay array consisting of DNA-origami antijunctions. The black strand is the scaffold. Unpaired scaffold loops are placed at the edges of the array. The 1^{st} (designated Red) confirmation on top with the corresponding 2^{nd} (designated Green) conformation on the bottom.

FIG. 3A illustrates multiple conformations that result from assembly of a DNA-brick relay array: “1^{st} array” conformation (designated Red), “2nd array” conformation (designated Green), and “mixed array” conformations, which contains units with 1^{st} conformation, 2^{nd} conformation, and intermediate conformation from one-pot assembly of 42-bp DNA-brick relay arrays. The intermediate units (corresponding to a higher-energy, unstable state) form diagonal seams that bridge together the 1^{st} confirmation units and the 2^{nd} confirmation units.

FIG. 3B illustrates a proposed, simplified energy landscape of assembly. The units assemble into the two global energy minima corresponding to the 1st array conformation and the 2nd array conformation, and local energy minima corresponding to the mixed array conformations.

FIG. 3C illustrates assembly of the 20×4 42-bp DNA-brick relay array results in two (1^{st} confirmation array and 2^{nd} confirmation array) dominant products in the agarose gel.

FIG. 3D shows AFM images of mixed array conformations of the 20×4 42-bp DNA-brick relay array.

FIG. 3E shows the 20×8 42-bp DNA-brick relay array is used as a “canvas” to generate relay arrays of different sizes and aspect ratios.

FIG. 3F shows percentages of 1^{st} array conformation, 2nd array conformation, and mixed array conformation of m×n relay arrays.

FIG. 3G shows numbers (n) of 1^{st} triggers (Rn) and 2ng triggers (Gn) shift the assembly result of the 11×4 42-bp DNA-brick relay array.

FIG. 3H shows removal of units from the 11×4 42-bp DNA-brick relay array alters the assembly result.

FIG. 3I shows a combination of unit removal and addition of eight 1st triggers affects the assembly result. Scale bars, 50 nm.

FIG. 3J shows preferred assembly results of the 11×4 42-bp DNA-brick relay arrays by programming the four base pairs at four-way junctions.

FIG. 4A illustrates section of the 11×4 52-bp DNA-origami relay array. Black strands denote the scaffold. Grey strands denoted the staples.

FIG. 4B illustrates toehold extensions added to both ends of a staple strand.

FIG. 4C illustrates a lock strand hybridized with the two toeholds to lock the unit in the 1^{st} conformation.

FIG. 4D illustrates control of the transformation pathway using shape design.

FIG. 4E illustrates transformation of a closed design is more cooperative. A stable conformation in which all units are partially open was observed at 40° C.

FIG. 5 shows a cross-section of a strand diagram and illustrates three-dimensional 42-bp DNA-brick relay array. The crossovers sequences connect helices horizontally and helices vertically. In a one-pot assembly, the design yielded detectable structures in TEM images. The TEM images show two orientations (helices parallel to the TEM grids) of each structure.

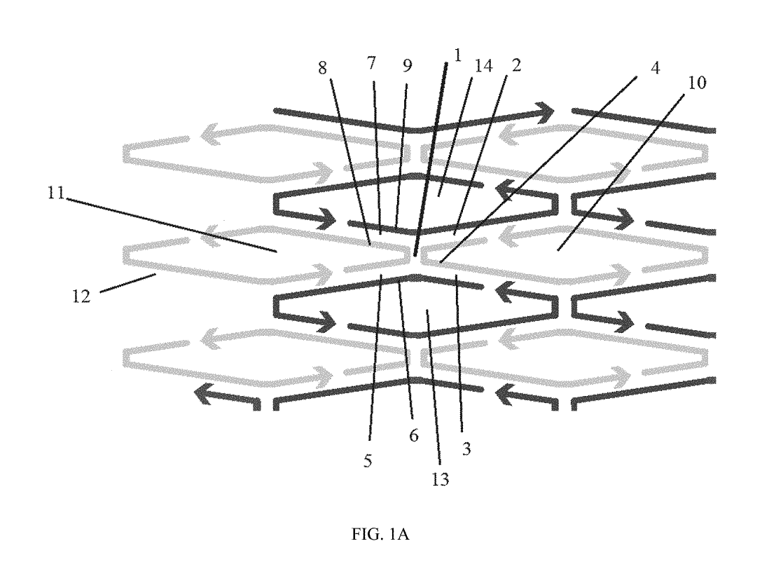

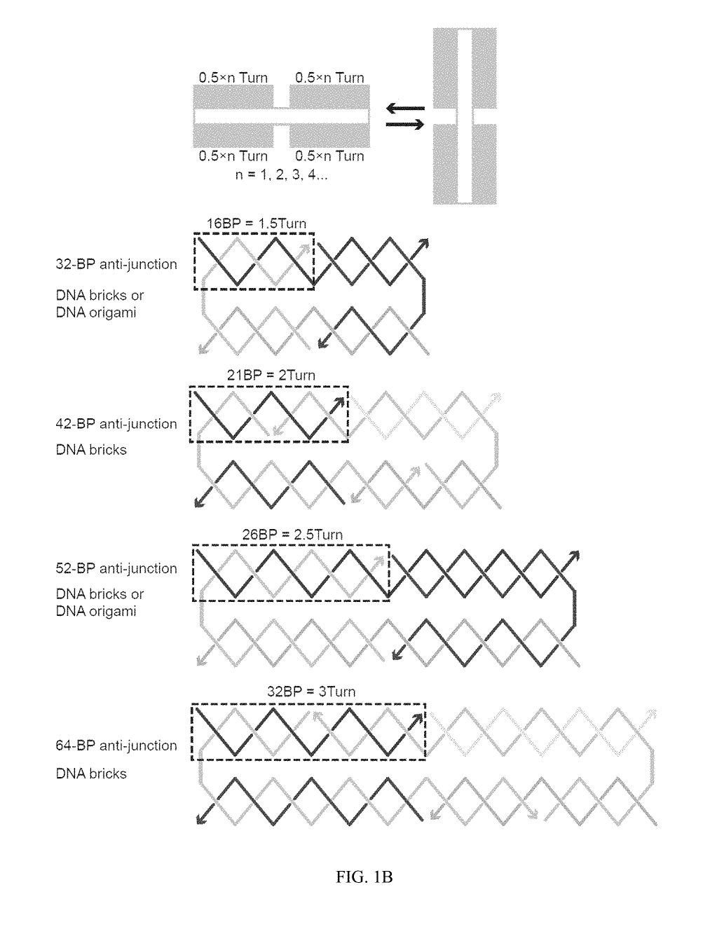

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Prior to describing the various embodiments, the following definitions are provided and should be used unless otherwise indicated. Further, headings provided herein are for convenience only and do not interpret the scope or meaning of the claims.

Unless the context requires otherwise, throughout the specification and claims which follow, the word “comprise” and variations thereof, such as, “comprises,” “comprising” “including,” “containing,” or “characterized by,” are to be construed in an open, inclusive sense, that is, as “including, but not limited to” and does not exclude additional, unrecited elements or method steps. By contrast, the transitional phrase “consisting of excludes any element, step, or ingredient not specified in the claim. The transitional phrase “consisting essentially of limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of or “consisting essentially of.”

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

In certain embodiments, the intersecting double helix arrays disclosed herein are produced from individual polynucleotides or DNA origami. The produce double helix arrays may be used as a building material to make nanoscale shapes and devices using handle domains, molecular latches, bound moieties, and combinations thereof. In general, DNA origami contain one or more long, “scaffold” DNA strands and a plurality of rationally designed “staple” DNA strands. The sequences of the staple strands are designed such that they hybridize to particular portions of the scaffold strands and, in doing so, direct the scaffold strands into a particular shape. Methods useful in the making of DNA origami structures can be found, for example, in Rothemund, P. W., Nature 440:297-302 (2006); Douglas et al., Nature 459:414-418 (2009); Dietz et al., Science 325:725-730 (2009); and U.S. Pat. App. Pub. Nos. 2007/0117109, 2008/0287668, 2010/0069621 and 2010/0216978, each of which is incorporated by reference in its entirety. Staple design can be facilitated using, for example, CADnano software, available at http://www.cadnano.org.

### Handle Domains

In certain embodiments, the intersecting double helix arrays disclosed herein include elements bound to a surface of its structure (e.g. molecular latches, handles, tethered moieties, etc.). Such elements can be precisely positioned on the array through the use of 5′ and 3′ polynucleotide overhangs, e.g., extended staple strands that include a domain having a sequence that does not hybridize to the scaffold strand. The additional elements can be directly or indirectly attached to such polynucleotides and staples. As used herein, the term “directly bound” refers to a nucleic acid that is covalently bonded an entity. In contrast, the term “indirectly bound” refers to a nucleic acid that is attached to an entity through one or more non-covalent interactions.

In some embodiments, the intersecting double helix arrays includes a polynucleotide having extensions capable of binding to a moiety (termed a “handle” domain). In such embodiments, such polynucleotide can be synthesized to have at least two domains, a hydrogen bonding domain and a handle domain. The hydrogen bonding domain is a sequence of the polynucleotide that hybridizes to the double helix array to contribute to the formation and stability. The handle domain contains additional nucleic acid sequence that is not necessary for the creation of the array.

In some embodiments, the handle domain can be directly bound to a moiety. For example, the handle domain can be synthesized with a particular moiety attached, or covalently bonded to a moiety prior to or following its incorporation into the array structure.

In some embodiments, the handle domain can be indirectly bound to a moiety. For example, before, during or after the formation of the double helix array, the handle sequences can available to be hybridized by oligonucleotides having a complementary DNA sequence. Thus, such polynucleotides can be indirectly bound by hybridizing the handle domain to another nucleic acid that has a nucleic acid sequence complementary to the handle and that is itself either directly or indirectly bound to a particular moiety.

The polynucleotides of intersecting double helix arrays can be selected such that a handle domain is positioned on any surface of the array, including inner surfaces and outer surfaces. In certain embodiments, the polynucleotides are selected such that a handle domain is positioned on an inner surface of the double helix array, e.g., when the double helix is folded into a tube structure. Handle positioned on an inner surface of double helix array and moieties bound by such handles are sequestered, and therefore sterically precluded from interacting with external element. Inner surface positioned handles are useful, for example, for preventing bound biologically active moieties from interacting with elements inside the tube structure.

In certain embodiments, the polynucleotides are selected such that a handle domain is positioned on an outer surface of the double helix array, e.g., when the double helix is folded into a tube structure. Such handles can be bound by, for example, molecular entities useful for delivery, detection or recapture of the double helix array. In some embodiments, for example, such outer handles can be bound by polyethylene glycol (PEG), carrier proteins, or other moieties capable of protecting the double helix array from a physiological environment. In certain embodiments, such outer handles can be bound by a detectable moiety, such as a fluorophore or a quantum dot. In certain embodiments, such outer handles can themselves facilitate the recapture of the double helix array, or can be bound by moieties that facilitate the device's recapture, such as antibodies, epitope tags, biotin or streptavidin.

### Molecular Latches

In some embodiments, the polynucleotides of the intersecting double helix arrays are selected such that the array held in a particular conformation by a molecular latch. In general, such latches are formed from two or more polynucleotide stands, including at least one polynucleotide strand having at least one stimulus-binding domain that is able to bind to an external stimulus, such as a nucleic acid, a lipid or a protein, and at least one other polynucleotide strand having at least one latch domain that binds to the stimulus binding domain. The binding of the stimulus-binding domain to the latch domain supports the stability of a present conformation of the array. The contacting of one or more of the stimulus-binding domains by an external stimulus to which it can bind displaces the latch domain from the stimulus-binding domain. This disruption of the molecular latch weakens the stability of the present conformation and may cause the array to transition to another conformation. In certain embodiments, this conformational change may result in previously sequestered moieties becoming externally presented and thereby rendering them capable of exerting a biological effect.

In certain embodiments, the polynucleotide of intersecting double helix arrays are selected such that the array includes multiple molecular latches. For example, in certain embodiments, the array includes two molecular latches. In some embodiments the various molecular latches may recognize different external stimuli, while in certain embodiments they recognize the same external stimuli. In certain embodiments, multiple external-stimuli binding domains and/or latch domains may be present on a single polynucleotide. In some embodiments, a stimuli-binding domain or a latch domain may span multiple polynucleotides which come together when the stimulus binds. In some embodiments, a stimuli-binding domain may bind multiple latch domains, or multiple stimuli-binding domains may bind a single latch domain.

The external stimulus to which the stimulus-binding domain can be any type of molecule including, but not limited to, a protein, a nucleic acid, a lipid, a carbohydrate and a small molecule. In certain embodiments, the external stimulus is expressed by a particular population of cells to be targeted by the array. In such embodiments, the external stimulus may be present on or near the surface of the targeted cell population. For example, in certain embodiments the external stimulus is a cancer cell-specific antigen. In some embodiments the external stimulus is a molecule that is able to specifically bind to a particular population of cells. For example, the external stimulus can be a moiety bound to an antibody specific for an antigen expressed by a particular population of cells (e.g. a cancer cell-specific antigen).

In certain embodiments, the external stimulus is a nucleic acid. In such embodiments, the stimulus-binding domain can have a nucleic acid sequence that is able to hybridize to at least a portion of the external stimulus. In certain embodiments, the stimulus-binding domain may include a sequence that is perfectly complementary to the sequence of the external stimulus or a portion of the sequence of the external stimulus. In some embodiments, however, the sequence of the stimulus-binding domain is less than perfectly complementary to the sequence of the external stimulus, but is still able to hybridize to a sequence of the external stimulus under certain conditions. Thus, in certain embodiments the sequence of the stimulus-binding domain is at least 10%, 20%, 30%, 40%, 50%, 60%, 65%, 70%, 75%, 80%, 85%, 90% or 95% complementary to a sequence of the external stimulus.

In some embodiments, the stimulus-binding domain further comprises a single-stranded toehold when bound to the latch domain. In some embodiments, the stimulus-binding domain comprises a non-nucleic acid moiety that is able to bind to an external stimulus. For example, in certain embodiments the stimulus-binding domain comprises an antibody, an antibody fragment, a protein or a peptide that is able to bind to an antigen or ligand present on or near the surface of a targeted cell. In such embodiments, the latch domain would comprise a moiety (such as an antibody, an antibody fragment, a protein or a peptide) that is also able to bind to the stimulus-binding domain. As with the aptamer domains, the binding of the stimulus to the stimulus-binding domain causes the displacement of the latch domain from the stimulus-binding domain. This displacement can either be a direct displacement (e.g., the stimulus and the latch domain bind the same epitope of the stimulus-binding domain), or can be indirect displacement (e.g. the binding of the stimulus to the stimulus-binding domain causes a conformational change in the stimulus-binding domain such that the latch domain is no longer able to bind).

In some embodiments, the intersecting double helix array may further include a “support” polynucleotide having a single-stranded toehold domain. A locking polynucleotide may be selected such that the presence of the locking polynucleotide on the array, e.g., by binding to the toehold domain, prevents transition of the array from one conformation to another conformation (e.g. from a first configuration to a second configuration or from a second configuration to a first configuration). In some embodiments, the intersecting double helix array may include a plurality of locking polynucleotides and supporting toehold domains. For example, in some embodiments the array will include 2, 3, 4, 5, 6, 7, 8 or more locking polynucleotide. Further contacting a locking polynucleotide with an oligonucleotide having a complementary sequence displaces the locking polynucleotide form the toehold and the array, thereby “unlocking” the device and permitting it to transition to another conformation if provided with the appropriate external stimulus. In certain embodiments, the single-stranded toehold domain of the locking polynucleotide extends from the inner surface of the array. In some embodiments, the single-stranded toehold domain of the locking polynucleotide extends from the outer surface of the array.

In some embodiments, the polynucleotides of the intersecting double helix arrays are selected such that the array emits one or more different detectable signals depending upon its conformation. For example, the array may include at least one polynucleotide bound to a fluorophore (a “signal polynucleotide “) and at least one polynucleotide bound to a quencher (a “quencher polynucleotide”). The binding of polynucleotides to fluorophores or quenchers can be either direct or indirect. The polynucleotide strands of the double helix array can be selected such that the transition from one conformation (e.g., a first configuration) to another conformation (e.g., a second configuration) changes the distance between at least one fluorophore and at least one quencher, thereby changing a signal output of the device.

### Bound Moieties

In certain embodiments, polynucleotide strands of the intersecting double helix array are selected such that one or more handle domains are positioned on the inner surface of the array. In certain embodiments, at least some of those handle domains are bound by a moiety. Such a moiety can be attached to the handle domain using any method known in the art. For example, the moiety can be covalently bonded to handle domain. The moiety can also be indirectly attached to the handle domain by, for example, hybridizing to the handle domain of a polynucleotide strand, as described above.

In certain embodiments, the intersecting double helix array includes a plurality of internally positioned handle domains. In such embodiments, the handle domains can all bind to a single type of moiety, or can bind to a plurality of distinct moieties. In some embodiments, the handle domains bind to a plurality of distinct moieties in a predefined a stoichiometric ratio. Such stoichiometric and spatial control of moiety binding is useful in, for example, the synergistic delivery of multiple biologically active moieties for combinational drug therapy.

In certain embodiments, the moiety bound to the handle of the intersecting double helix array may include an antibody. As used herein, the term “antibody” includes full-length antibodies and any antigen binding fragment (i.e., “antigen-binding portion”) or single chain thereof. The term “antibody” includes, but is not limited to, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

As used herein, the phrase “antigen-binding portion” of an antibody, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include (i) a Fab fragment, a monovalent fragment consisting of the V_{H}, V_{L}, CL and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{H} and V_{L} domains of a single arm of an antibody, (v) a dAb fragment (Ward et al., (1989) Nature 341:544 546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); see e.g., Bird et al. (1988) Science 242:423 426; and Huston et al. (1988) Proc. Natl. Acad. Sci. USA 85:5879 5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The present intersecting double helix arrays permit use of thermally labile moieties as payload, provided that the array assembled in a confined orientation includes an opening large enough for the moiety to enter the array, e.g., tube and become bound. Assembly may utilize a temperature high enough to denature DNA. Denaturation may start at temperatures as low as 50 or 60 degrees Celsius. Denaturation is typically carried out at a temperature in the range of 60-95 degrees Celsius. An annealing mixture can optionally be added later, once the reaction has cooled.

### Design of DNA Relay Arrays

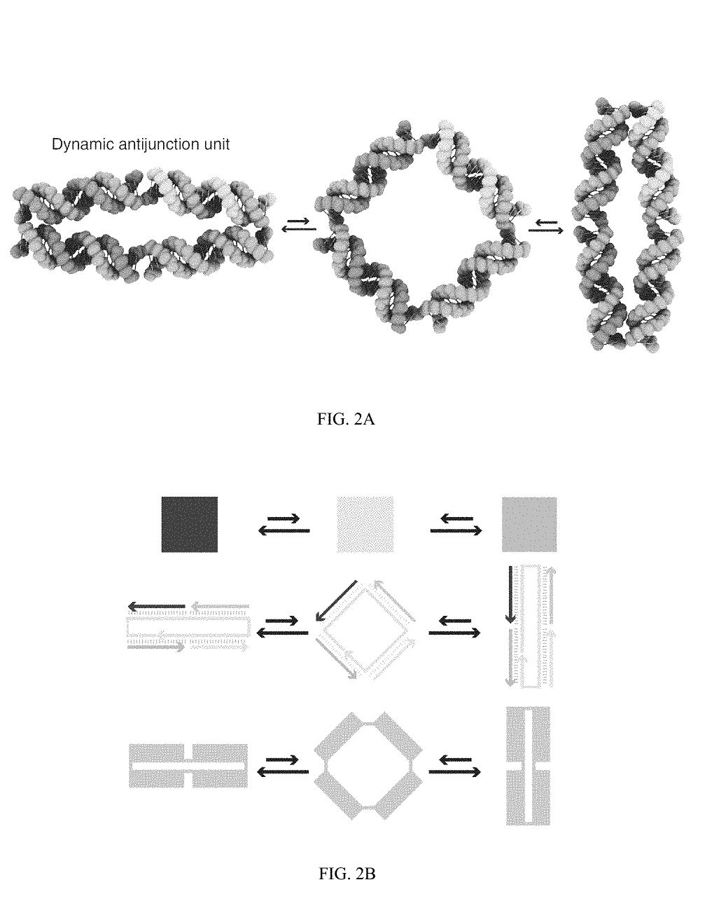

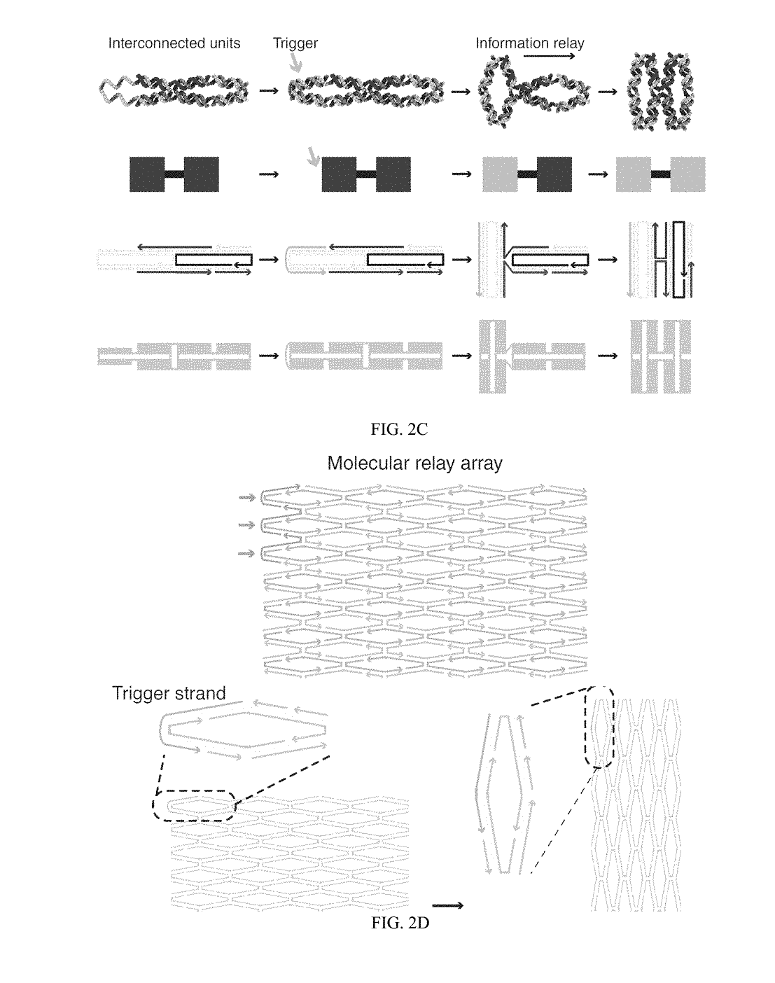

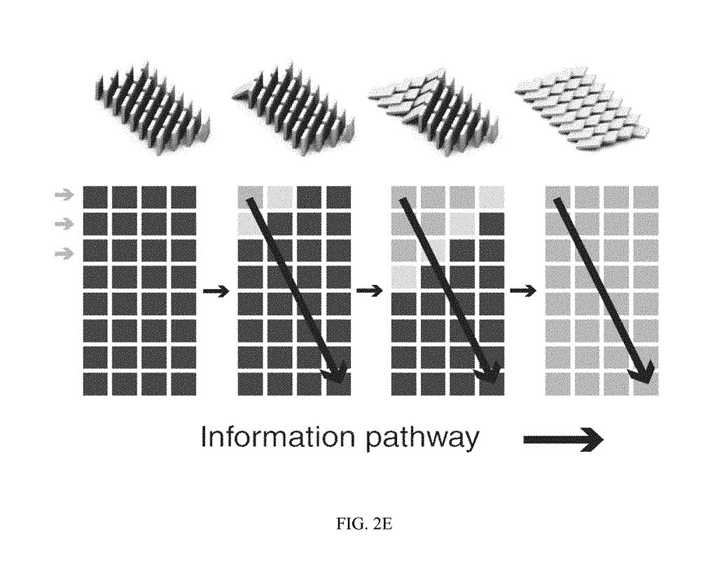

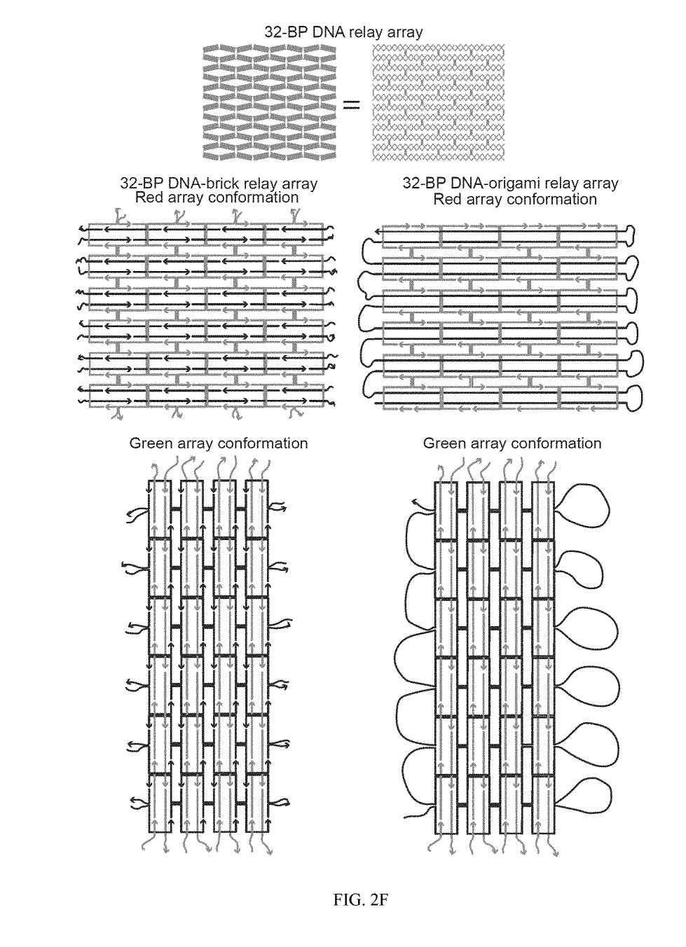

Small dynamic DNA units called “antijunctions” were used to build large, scalable, reconfigurable DNA structures. An antijunction unit contains four DNA duplex domains of equal length and four dynamic nicking points (FIG. 2A and 2B; note that the strand in gray contains a static nick, which does not change during reconfiguration). This small construct can switch between two stable conformations driven by base-stacking, through an unstable open (including partially open) conformation. Each duplex is 0.5×n turns (n=1, 2, 3, 4 . . . ) in length. An antijunction is classified by the distance between two opposite dynamic nicking points (i.e., a 42-bp antijunction). In a connected network, the conformational information of an antijunction can pass to its closest neighbors, introducing subsequent conformational change of the neighboring antijunctions (FIG. 2C). The transformation of an individual antijunction unit can be induced by adding a trigger DNA strand that forms a continuous duplex on one edge of the unit. The design feature of trigger strands is that each trigger strand removes a mobile nick point from an antijunction unit. After the conformational switch of the triggered unit, the interface between the two neighboring units becomes a high-energy open conformation, leading to a transformation in the neighboring unit to the same conformation as the already transformed unit. This process is driven by the reduction of free energy, caused by the formation of an additional base-stacking interaction at the connection point.

A 2D molecular DNA relay array was built via self-assembly of the antijunctions (FIG. 2D). A relay array can transform from one array conformation (e.g., antijunctions 1^{st} conformation) to another array conformation (e.g., antijunctions are in the 2^{nd} conformation). The array transformation follows specific pathways, depending on the array's geometry and binding locations of trigger strands. For instance, if the trigger strands were added to the units (FIG. 2D and 2E) at a corner, the relay would undergo a step-by-step conversion from a 1^{st} array conformation to a 2^{nd} array conformation via a diagonal pathway (FIG. 2E).

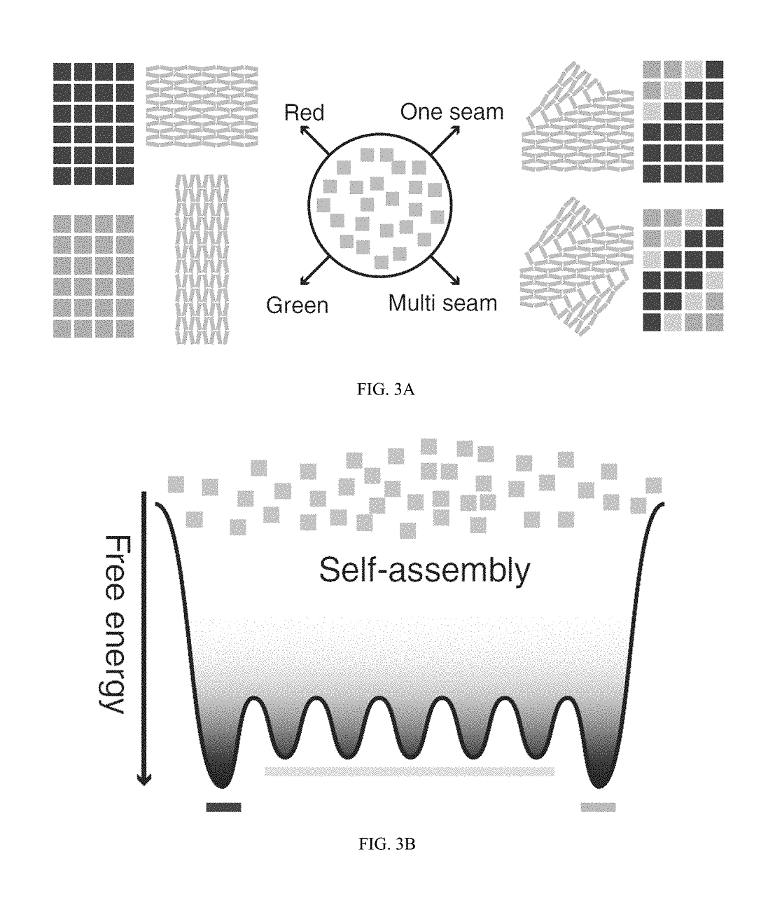

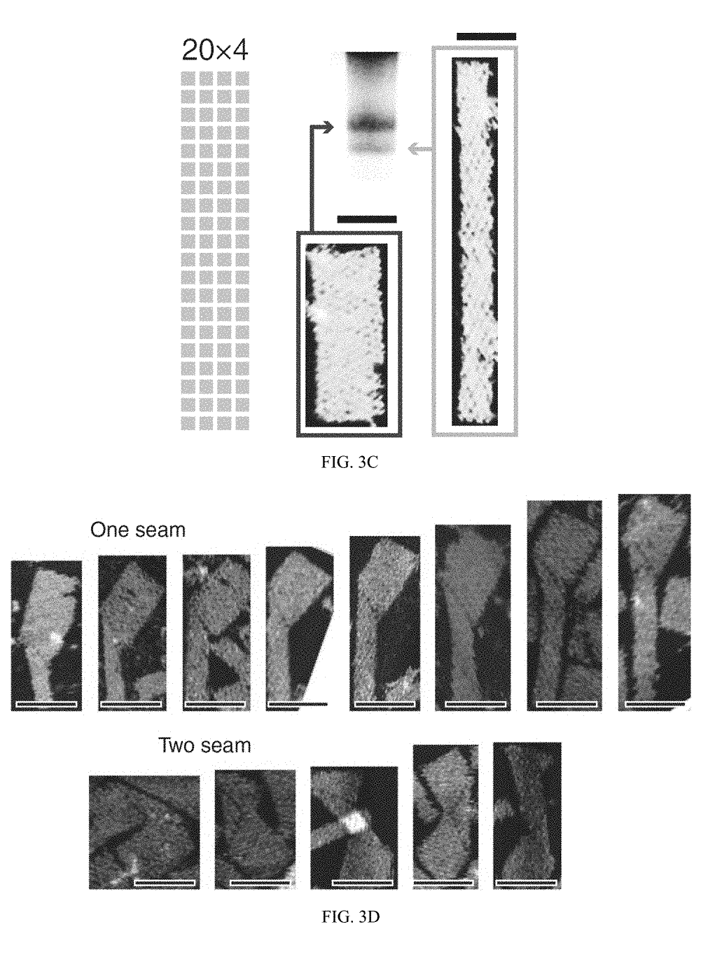

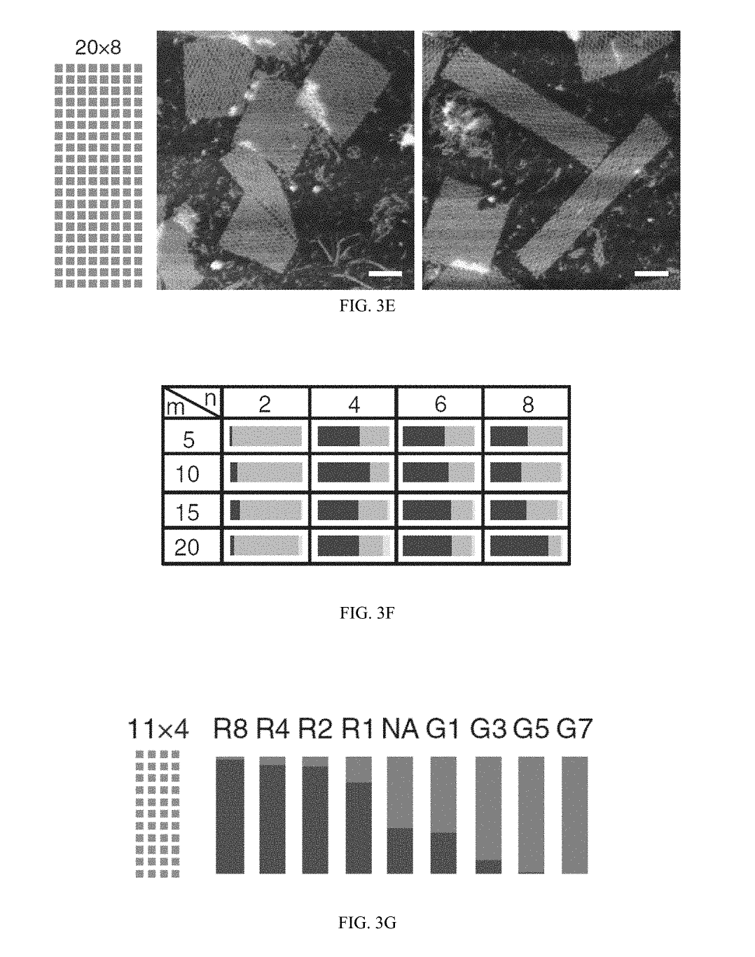

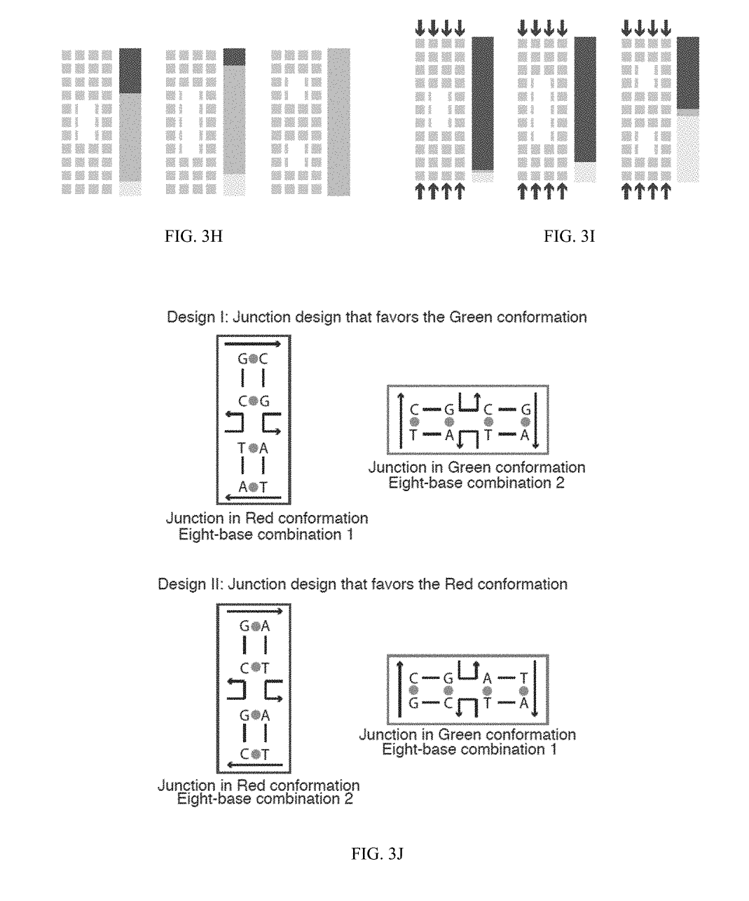

DNA relay arrays can be constructed with both noncanonical DNA bricks (single-stranded modular DNA units) and DNA origami (FIG. 2F). Owing to the constraint of the continuous scaffold, a DNA-origami antijunction must be an odd-number-turn antijunction (e.g., 32-bp antijunction). In comparison, a DNA-brick antijunction can be either an odd-number-turn antijunction (e.g., 32-bp antijunction or an even number-turn antijunction (e.g., 42-bp antijunction). One of the array conformations of the DNA-brick arrays is arbitrarily assigned as the 1^{st} array conformation (designated Red), and the other is assigned as the 2^{nd} array conformation (designated Green) (FIG. 3A). For DNA-origami relay arrays, the conformation where the scaffold does not cross between DNA helices within the array is assigned as the 1^{st} array conformation, and the other conformation is assigned as the 2^{nd} array conformation.

### One-Pot Assembly of DNA-Brick Relay Arrays

The transformation pathway of a DNA relay array is expected to be dictated by the stable conformations corresponding to local energy minima. To investigate these local energy-minimum states, the self-assembly of rectangular 42-bp DNA-brick relay arrays via were studied by one-pot isothermal assembly. The results revealed that the most dominant conformations were the 1^{st} array conformation and the 2^{nd} array conformation. In addition to the two dominant conformations, which should correspond to the two lowest-energy states, many mixed conformation arrays were observed that consist of both regions of 1^{st} array conformation antijunctions and regions of 2^{nd} array conformation antijunctions. The 1^{st} array conformation antijunction regions and the 2^{nd} array conformation antijunction regions were always bridged by a diagonal seam(s) that contains open intermediate antijunctions. These intermediate antijunctions are unstable by themselves, but can exist in an array structure when they are flanked by 1^{st} array conformation antijunctions and 2^{nd} array conformation antijunctions (FIG. 3A). These arrays with mixed antijunction conformations are called “mixed array” conformations, which correspond to local energy minima in the assembly (FIG. 3B).

Native agarose gel electrophoresis of a rectangular 20 unit by 4 unit (20×4) 42-bp DNAbrick relay array revealed two product bands that correspond to the 1^{st} array conformation and the 2^{nd} array conformation, respectively (FIG. 3C). The 2^{nd} array conformation showed greater mobility than the 1^{st} array conformation. The mixed array conformations contain many different shapes that do not migrate as a single band, but were observed in atomic force microscopy (AFM) images of unpurified samples (FIG. 3D). An 11×4 42-bpDNA-brick relay array was used to test optimal assembly conditions. The best yield was observed when the array was assembled at 51.3° C. isothermally, in a Tris-EDTA (TE) buffer containing 10 mM, MgCl₂. Addition of single stranded poly-T extensions around the boundary of the array further improved the yield, presumably due to the poly-T's function of mitigating unwanted aggregation.

### Regulation of Assembly of DNA-Brick Relay Arrays

To understand how size and aspect ratio affect the assembly of DNA relay array, the one-pot assembly of a group of rectangular 42-bp DNA-brick relay arrays was tested. The largest structure is a

20×8 DNA-brick array consisting of randomly generated ˜14,000 bp (FIG. 3E). In total, 16 DNA brick relay arrays with different sizes and aspect ratios were generated by using the 20×8 relay array as a molecular canvas (FIG. 3F). Native agarose gel electrophoresis and AFM images each four-way junction. The assembly results showed a strong correlation between the junction sequences (and thus the base stacking) and the 1^{st}/2^{nd} confirmation (R/G) ratios. The 2^{nd} array was the dominant conformation (R/G=0.24) for design I, while the 2^{nd} array was the dominant conformation (R/G=3.0) for design II (FIG. 3J).

### Transformation of DNA-Origami Relay Array

To identify a suitable design for our study of DNA array transformation, different DNA brick relay arrays and DNA-origami relay arrays were compared. Overnight room-temperature incubation of 1^{st} trigger strands with a preassembled 11×4 42-bp DNA-brick relay array, an 8×5 52-bp DNA-brick relay array, and a 10×4 64-bp DNA-brick relay array did not convert a noticeable percentage of 2^{nd} array conformation to 1^{st} array conformation. The arrays were then incubated at higher temperatures or in solutions containing higher concentrations of formamide to accelerate the transformation kinetics, but the DNA-brick relay array started to show damage at 55° C. or 40% formamide before noticeable transformation was observed.

DNA-origami relay arrays are more resilient to denaturing conditions. An 11×7 32-bp DNA origami relay array and an 11×4 52-bp DNA-origami relay array we assembled with a p7560 scaffold. Both arrays resulted in only the 1st array conformation. It is unknown how the scaffold (about half the molecular weight of the whole array) might affect the formation and transformation of the DNA-origami relay array. The 52-bp DNA-origami relay array was selected for real-time transformation, as subsequent studies revealed that this relay array is easiest to transform and is convenient for image analysis.

Transformation of preassembled 11×4 52-bp DNA-origami relay array in the 1^{st} conformation was initiated by the addition of 22, 2^{nd} trigger strands. The arrays initially transformed to mixed conformations at 45° C., then to ˜100% 2^{nd} array conformations at 55° C. Analysis of the mixed array conformations revealed that the dominant pathway of transformation is diagonal, consistent with the results for DNA-brick relay arrays. Based on the results of the transformation from the 1st array conformation to the 2^{nd} array conformation, an assumption was made that the 1st unit conformation was slightly favored over the second unit conformation in the p7560 DNA-origami relay array, resulting in a tilted energy landscape for the transformation process.

Real-time AFM was used to study the in situ single-molecule transformation of the DNA relay arrays. Within a 30-min scan, multiple array transformations were observed. The transformation appeared to be a stochastic process: Most of the transformations started from a corner and propagated through a diagonal pathway, while a smaller number of transformations were initiated from the edges and propagated along a “swallowtail” pathway. In rare cases, transformation of an array was also observed being initiated at multiple locations.

The transformation of the 52-bp DNA-origami relay array is reversible. Using modified trigger strands with toehold extensions and corresponding release strands, multiple conversions between the 1^{st} array conformation and the second array conformation were demonstrated. The 2nd array conformation has two product bands: The upper band and the lower band correspond to the folded structure [due to the tension generated by the free scaffold] and open structure, respectively. The “open” 2nd conformation is more dominant than the “folded” 2^{nd} conformation after the initial assembly. However, after the array's transformation to 1^{st}, and then back to 2nd, nearly 100% of arrays turned into the folded 2^{nd} conformation.

The kinetics of the transformation can be accelerated by either increasing temperatures or using higher concentrations of formamide likely because of the reduced energy needed to break the base-stacking interactions under these denaturing conditions [e.g., increasing the temperature reduces the base stacking energy from an average of −5.2 kJ/mol at 32° C. to −2.9 kJ/mol at 52° C.]. The real-time transformation was acquired by using DNA-origami relay arrays in a 10% formamide solution. The real-time transformation of an 11×4 32-bp DNA origami array was observed at 65° C. using a temperature-controlled AFM. However, the AFM images are much noisier at such a high temperature. The kinetics of transformation can also be increased by the mechanical disturbance induced by the AFM tip. The transformations occurred at higher frequencies when the sample was subjected to the contact force from the AFM tip. This AFM tip-enhanced transformation was confined to only the scanned area. This phenomenon may provide a means to manipulate our dynamic DNA arrays at specific locations.

### Regulation of Transformation in DNA Relay Arrays

With a better understanding of the factors affecting transformation, a study was extended to control the transformation of the DNA-origami relay arrays. Transformation could be initiated at selected locations, blocked, and controlled using arrays with different shapes. The transformation can be initiated at prescribed locations on the 11×4 52-bp DNA origami relay array. Using real-time AFM, initiation of transformation was demonstrated from a corner or from the middle of an edge with five 2^{nd} trigger strands and subsequent propagation of the new conformation. Both the number and locations of trigger strands affect the initiation of array transformation and the degree of transformation. With three or fewer trigger strands added in a corner, the transformation process could not be initiated. Increasing the number of trigger strands to six or eight triggered partial conversion to mixed array conformations, with a small number of 2^{nd} array conformations. The addition of 11 2^{nd} trigger strands led to full transformation to the 2^{nd} conformation for most of the arrays. Adding trigger strands to the corners appeared to be more effective at inducing transformation than adding the same number of triggers to the edges. In addition, adding both 1^{st} trigger and 2^{nd} trigger strands can lead to transformation to specific mixed array conformations. The transformation efficiency of preassembled arrays were compared using a one-pot assembly in the presence of triggers. The results showed that conversion was typically more complete under the one-pot assembly condition.

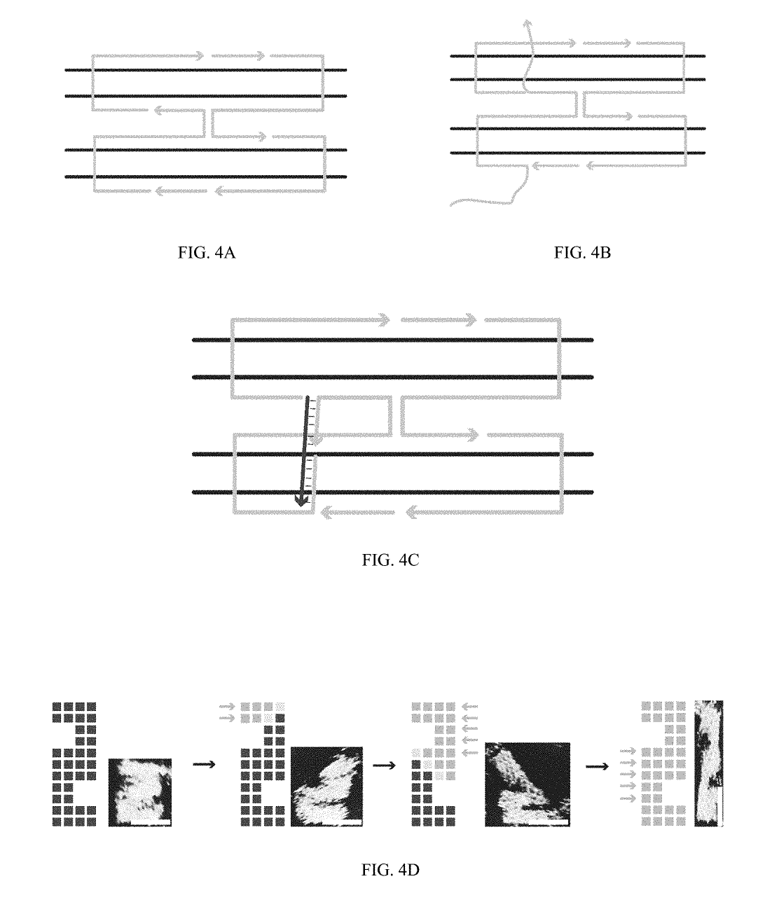

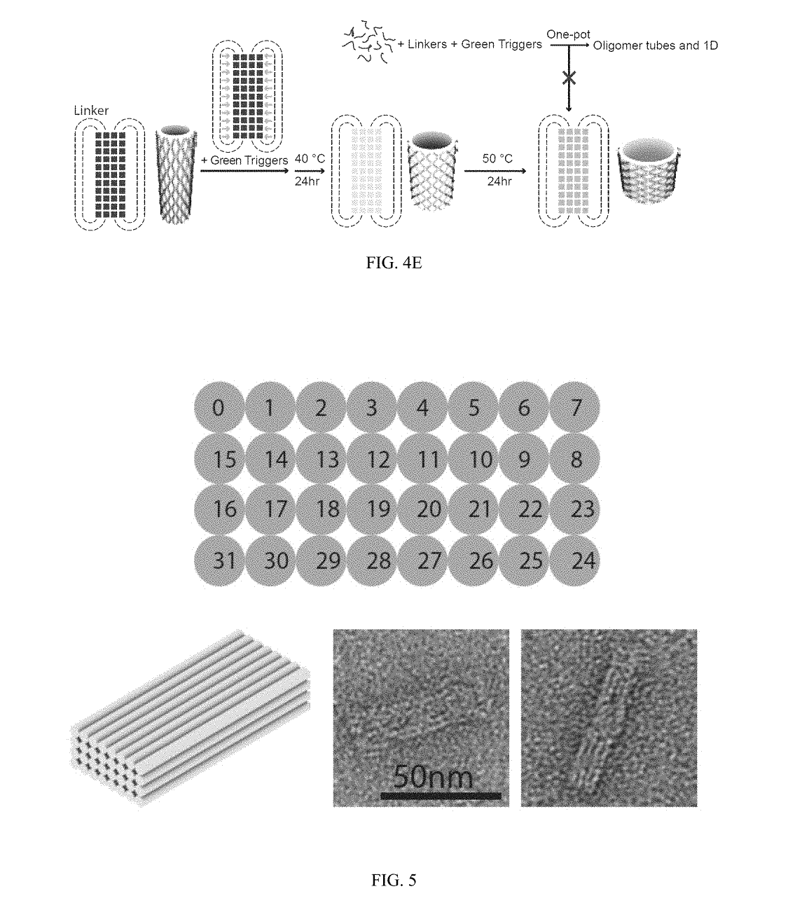

Two strategies were explored to turn off or turn on transformation at selected locations by blocking or resuming the information pathways between units. In the first approach, an “off” function was created by removing one unit from the relay array, which creates a local energy minimum that traps the array transformation. Reintroduction of the missing unit enables the transformation to escape the trap and proceed. Blocking and resuming of transformation at different locations on the array using this strategy was demonstrated. In a second approach, the “off” function can also be achieved by using a “lock” strand (FIG. 4A-C). This strand binds to single-stranded DNA extensions from two neighboring units, effectively locking the units into a fixed conformation (FIG. 4C).

Information relay in a DNA relay array can also be programmed by removal or addition of antijunction units. Using the 11×4 52-bp DNA origami relay array as a canvas, a “2” shaped array was produced by removing eight units. (FIG. 4D). After the initiation of transformation at the top corner, this array transformed in a three-step process: The addition of two initial corner triggers transferred the array only up to the top-right corner; five additional triggers were added to the top-right corner to push the transformation about halfway through the array; finally, the array transformation was completed after the subsequent addition of another five triggers. Each step was verified in AFM images.

Spontaneous transformation of both DNA brick relay array and DNA-origami relay array without addition of triggers was occasionally observed during the AFM scan in the presence of 10 to 30% formamide. The transformation of DNA-origami relay arrays is influenced by connecting the arrays into monomer tubes, and into oligomer 1D chains and tubes. The 1^{st} triggers were substituted for connector strands that link the top and bottom edges of the 11×4 52-bp DNA-origami relay array. The relay array assembly with the connectors resulted in a 1^{st}-conformation nanotube, which could convert to a 2^{nd}-conformation tube after addition of 22, 2^{nd} trigger strands (FIG. 4E). In comparison to the 2D relay arrays, unit conversion in the nanotube was more cooperative. A two-step process was observed: first, the tube was converted to a stable state in which most units appeared partially open at 40° C.; then, the tube was fully converted to a 2^{nd}-conformation tube at 50° C. (FIG. 4E). By contrast, one-pot assembly of the array with both the connectors and 2^{nd} triggers did not produce 2^{nd}-conformation tubes.

### Reconfiguration of DNA Molecular Arrays Driven by Information Relay

Described herein are programmable molecular information cascades formed by DNA arrays, which simulate some of the key aspects of complex biological signaling cascades, such as initiation, propagation, and regulation observed in signaling cascades initiated by T cell receptor binding. These large, scalable DNA arrays are analogous to a molecular “domino array”: the step-by-step transformation propagates through the interconnected DNA units via specifically prescribed pathways. The reversible transformation of a DNA array can be initiated at designated locations, and follows pathways precisely controlled by programming the shape of the array or by adding molecular switches that block and then resume the information relay between units. This dynamic behavior can be regulated by the shapes and sizes of arrays, by external factors (e.g., temperature), by the initiation of transformation at selected units, and by the information propagation pathways.

In certain embodiments, this disclosure contemplates extending the DNA relay arrays to 3D spaces, larger sizes, more intricately shaped designs, and more complex dynamic behaviors. In FIG. 5, a small 3D DNA-brick relay array design is shown, which consists of two sets of dynamic junctions, perpendicular to each other. This 3D DNA-brick relay array produced three detectable conformations, confirmed by TEM images. Canonical non-reconfigurable 3D DNA-brick structures were produced up to ˜12,000 bp as reported in Ke et al. Science 338, 1177-1183. Assuming that reconfigurable structures with a similar size could be made, the structure would contain ˜142 42-bp antijunction units. Such large, intricate structures would enable demonstration of complicated transformation in 3D space. Hierarchical assembly of multiple DNA relay arrays can lead to construction of larger and more intricate designs.

The transformation propagation in the DNA relay arrays resembles crucial features of allosteric mechanisms that are observed in biological systems. Therefore, the arrays may serve as model structures to investigate and validate underlying mechanisms of allostery, or be used to design and construct allosteric metamaterials. The DNA relay arrays may also be used as a platform to analyze biomolecular interactions at a single-molecule level, by translating and amplifying the molecular interactions to conformational changes in the DNA relay arrays or to subsequent molecular events (e.g., chemical reactions, fluorescence resonance energy transfer, etc.). Rational sequence design may be used in DNA brick relay arrays to study the binding energy of the junctions, which could enable more sophisticated control of the assembly and transformation of the DNA relay arrays.

### EXAMPLES

### Isothermal Assembly

The folding of DNA origami or DNA bricks is highly cooperative. The folding rates of DNA origami or DNA bricks is elevated at constant temperature T_{fold}, whereas deviating from this optimal T_{fold} slows or entirely inhibits structure formation. Therefore, both DNA origami and DNA bricks can be assembled at constant temperature. However, initial denaturation of DNA strands with a heat shock (e.g. 5 min at 90° C.) is still necessary to resolve secondary structures in the template scaffold for DNA origami. Different DNA nanostructures may have different optimal T_{fold}, so it is necessary to determine the optimized isothermal condition for any new DNA nanostructure. To screen for the T_{fold}, DNA origami or DNA bricks can be folded at different constant temperatures (e.g. fold samples at every elevated 2° C. from 45° C. to 70° C.), then characterize the samples by agarose gel. Successful folding of samples will have clear bands. The gel bands can be further purified and the structure can be characterized by AFM. For 42-bp DNA-brick array, the optimized isothermal condition was 53° C. with 1 mM EDTA and 10 mM MgCl₂ for 18 hours.

### Thermal Annealing Assembly

To form DNA origami or DNA brick structures with high yield, the DNA strand components are typically annealed in an assembly favoring buffer by heating the mixture to a high temperature followed by incremental cooling over the course of several hours up to several days. For 52-bp and 64-bp DNA-brick arrays, an annealing process (95° C. for 5 mins, from 85° C. to 24° C. at the rate of 20 mins/° C.) was used with 50 mM Tris, 1 mM EDTA and 10 mM MgCl₂. For DNA origami relay arrays, the mixture of staple strands (final concentration: 100 nM of each strand) and the scaffold (final concentration: 10 nM) were mixed in 1×TE buffer, supplemented with 10 mM MgCl₂. The samples were then annealed for 10 hours using the following thermal annealing protocol: 95° C. for 5 mins, from 85 to 24° C. at the rate of 10 mins/° C.

### Purification

After folding the DNA nanostructures, excess staple DNA should be removed before AFM or TEM imaging. We include three techniques for DNA origami or DNA bricks purification and quality control in this protocol. PEG precipitation uses the depleting agent poly(ethylene glycol) (PEG) to precipitate the DNA samples and separate the folded objects from staple strands. PEG precipitation can concentrate the samples while adjusting the buffer and it is suitable for high throughput sample purification. Agarose gel electrophoresis is a common method for DNA nanostructure quality control and sample purification. The agarose gel separates DNA samples based on their electrophoretic mobilities. The quality of the gel bands provides a first impression of folding quality, and shows whether dimerization of DNA nanostructures occurred. The target DNA molecular arrays can be efficiently separated from the free staple DNA by excising a gel slice containing the species, followed by gel-extraction centrifugal filtering. Increasing the agarose gel concentration to ˜2.5% enables the separation DNA molecular arrays adopting different conformations. Separation of DNA nanostructures with molecular weight cut-off membranes offers residual-free separation but only works for smaller volumes compared with PEG precipitation due to limits in the size of filtration membrane. DNA molecular arrays are purified by filter with 100 kDa MWCO. Generally, ultrafiltration with molecular cut-off filters is an attractive method to separate DNA origami from free DNA strands quickly (≈30 min) and efficiently. Typically, the purification process can be accomplished with 3-6 centrifugations at 3000 g for 10 min. The DNA origami or DNA brick samples can be collected after flipping the filter into a new tube and further centrifugation.

### Regulation of DNA Array Transformation

Because the two dominant conformations of DNA relay arrays correspond two local minimum energy states, transformation of the DNA relay array from one conformation to another needs to overcome the energy barrier between the two states. Both elevated temperature and addition of formamide can assist in this process. Different DNA molecular arrays were identified using the two methods. Unfortunately, DNA brick arrays cannot be transformed with either elevated temperature or formamide. However, the 11×4 52-bp DNA origami relay array with a p7560 m13 scaffold can be successfully transformed with trigger strands in 55° C. or 10-40% formamide. The kinetics of the transformation can be accelerated by either increasing temperatures or using higher concentrations of formamide, likely due to the reduced energy needed to break the base-stacking interactions under these denaturing conditions. With a temperature-controlled Bruker AFM instrument, one can observe the transformation of a DNA relay array in real time. Alternatively, the denaturing agent formamide decreases DNA melting temperatures linearly by approximately 0.6° C. per % formamide in the buffer. The transformation with formamide can be achieved at room temperature, and thus it is more convenient for real-time imaging of DNA array transformation. In addition, the transformation can also be accelerated by AFM tip-enhanced scanning as the contact force from AFM tip provides mechanical energy which can aid in transformation.

With real-time AFM, one can track the information relay propagation pathway. The trigger strands can be added selectively at the corner or middle of the 11×4 52-bp DNA origami relay array. Besides the position of the trigger strands, the number of trigger strands can also influence the kinetics of the transformation. The transformation will not start with only three or fewer trigger strands.

The process of transformation is reversible and tunable. Employing modified trigger strands with toehold extensions and release strands, one can reversibly switch between red array conformation and green array conformation. In addition, we used two methods to turn on or off the transformation at desired locations. The first method is to remove one unit from the relay array to stop the transformation propagation. Interestingly, adding the missing unit resumed the transformation. In the second method, a “lock” strand binds to single-stranded DNA extensions from two neighboring units, thus preventing the propagation. The relay array can assemble in a red conformation tube by linking the top and bottom edges of DNA origami relay array. The nanotube can further convert into green conformation tube by adding green trigger strands.

Sequences of the 10 x 4 64-bp DNA-brick
relay array.
2,90 core
(SEQ ID NO: 1)
TACGGTTTTAGCATCAGAGGGTCCGGAACTAAGAAGGAGGTGATCTCG
TTGCTTTATGTCTTGA
2,154 core
(SEQ ID NO: 2)
GGATCACATTATCGCCGTTCTTGAACCCGTGTATCCATACTCTCGGAC
ACGCTATCGCGATACT
2,218 core
(SEQ ID NO: 3)
CTCAGGGACCCATAATTACGTCCGGCGGCGATGTCCAACCCGTGGTGG
CACTTGAACTTAAAGT
3,59 core
(SEQ ID NO: 4)
GAGTCATATGGCCCTGTCAACTGAAAGACCGGACGTGCGACGTCGTTT
GGATTTGCACGCTTCG
3,123 core
(SEQ ID NO: 5)
CACAACCGGACGTTCATGGAATTAGGAAGACCCGGGACCCCATGTCAG
ATAGAGTAGGTTGCTA
3,187 core
(SEQ ID NO: 6)
TCTATCTTAATCCTCCTGAGCGAATAGTTAAATACCCGCCACAGGGCC
GTGAACAAAGTGCAAG
4,90 core
(SEQ ID NO: 7)
TTGGCGTTCACTTGGCTTAGCTCCTAGAGTTTGCCTCGGTAGGTTAGA
CCGTACTCCATGATCA
4,154 core
(SEQ ID NO: 8)
TTGACGTGTAAGTCGGCCTGCTCGATCTACACGATGAAGCCCTCCCAA
TTAATAAGGCCCAACC
4,218 core
(SEQ ID NO: 9)
AGCATTAAACAGGTTATCACCTTTTCACTCCTGGACACGAGGCGGCGT
TTTCCATTGGACGGCC
5,59 core
(SEQ ID NO: 10)
GTGCACATCAGAGATGCTGGGAGAGATGCATGTCTGAGAACGGTTTTT
GGATACATCCAAACTG
5,123 core
(SEQ ID NO: 11)
CCCGTAGCATGGGGAGACGCAGAGAACGCAGTGAGCTGATTAGAAATA
GGGGACTCCGATCCAT
5,187 core
(SEQ ID NO: 12)
GCAGTGCCACAATTCGTTACCGAGTTCTTTAAACTCTCTCTATGGAGG
AAGAGGTGCCACCGTT
6,90 core
(SEQ ID NO: 13)
ATATCTGCCTAAGCTAGGAGCTCTAGCGATGGGGATTAGTGATGGCGG
TCTTTGGATCCTTATT
6,154 core
(SEQ ID NO: 14)
TCTAAACGCAGGGCCACCAGATACCCTACCACGCACCTGTGGCACATT
CTAGATCCCTGCGGTT
6,218 core
(SEQ ID NO: 15)
TAGGGGTGTGGAGTGCGACAGTTTGAATCGATATCTATCACAACCTTT
GGCAGCAGCACTGCCA
7,59 core
(SEQ ID NO: 16)
TGCGACAAGGTACACGGTTCTAAGGTTGGCGACATTTAATTAAATATG
CAAGCTCTAAATCGTC
7,123 core
(SEQ ID NO: 17)
ATCTCCTTTTTACCCATGTCTCTCTATGTCTCCAACATTTATTCGTGT
GCTTTCCATACATGCT
7,187 core
(SEQ ID NO: 18)
ACTCTTCGAAAATTTCGACCTCACCACTTTACCTTTTAAAAGCAGTTT
AGCTTCCCCACAGCTT
8,90 core
(SEQ ID NO: 19)
GGTGTCTTTCACCAATGTGTTCTGACCCCGTCACGCCAGGGACAGCGA
TTCTAGGAAACGTAAT
8,154 core
(SEQ ID NO: 20)
CTGCGTTAACGTCAACTCATTAGCGGACTATTGCCGTGTCTAACTTAC
GCAAAACGAATTGCTT
8,218 core
(SEQ ID NO: 21)
CACCTTACACCGTCAAGTGTCTTTATCCGCCTGTACCATTTGCACTGG
ATCGGTTGAGGGGAAG
9,59 core
(SEQ ID NO: 22)
GATTACCGTACGGTTTGTAATTTCCCTCCTCTCGAACATTAAAAACTT
CACACACCTTTGACAG
9,123 core
(SEQ ID NO: 23)
GCGACCAAAGTAGCTGACTCCTCAATGTAAGACAATTACTTGTGTAGT
AAAGTACCACGGGTAG
9,187 core
(SEQ ID NO: 24)
CCGTTTTCAATGCTGGACATCCGTTTCGTACTAGACCTGAGAACGCGC
TAAGGGGCCGATTAGT
10,90 core
(SEQ ID NO: 25)
TCTAGCAACGCCATCCTGGCGATAAATCTGTAGGACACTGAAACCGTT
GAAAAACACAGCGCCT
10,154 core
(SEQ ID NO: 26)
GTCTTAGATGAGCACGGGAGGTAAGCTGAGCCTACTCAATTTCCATGG
TGTACCCACATCAGCT
10,218 core
(SEQ ID NO: 27)
GAACATTTGATCCGGGTAGCCCCCACCATTCCTAGACTAATTGAATAT
GTGGTGCTCTTCACTT
11,59 core
(SEQ ID NO: 28)
CGTTGCTGTCGGCCGTGTATCGAACTATCACTAAGGCTGTGGGCATGG
GCGGCCTTAGACAGAG
11,123 core
(SEQ ID NO: 29)
CACGTGGAGGTAAGGCAGTCGACCACACGTACAACACGGAGTTCCTGT
GCATGAAACCCCACGC
11,187 core
(SEQ ID NO: 30)
TAAGGAGCCCCGTCCGACGACTCATGAACTTTAAGTGAATTTATGTTC
CCTTGCGTTAGCAACG
12,90 core
(SEQ ID NO: 31)
CGCGAGGGCACGCCGGTGAAGAACGCCAATAGACCTACTTGTTTCACA
CGTAACGGTACGTAGG
12,154 core
(SEQ ID NO: 32)
GATATGGACAAACAACTGCCCCCGGGCACCCTACAAGGGTGCACGCGA
TTATTCCACTGTGTCG
12,218 core
(SEQ ID NO: 33)
TCATGGCACTTAAGGCAATATAGGCGTATTAGCAAGGGAATCCCTGCT
CTTCCGTTGAAAACTT
13,59 core
(SEQ ID NO: 34)
GTAACAGATGTCACAGAATCGCCCCTCGTTATTCATACGGAGATACCA
GACGAGACCCGGCCTC
13,123 core
(SEQ ID NO: 35)
TTCAGTAGACCTGCTTGGGTAACATAACAAATTAGAATTCCTTCCTGT
CGGCTGCATGGTGGTT
13,187 core
(SEQ ID NO: 36)
TCATCAGGCGGTTTCGTTTTCGATCGGGGTAAAAGAGATAAGTTCAAC
ATGAATCTCGTCGTCA
14,90 core
(SEQ ID NO: 37)
AGGGCTGCCACGATTATCCCTACGACCGGTTACTGACCAGGGTTACAA
GCCATACATACTGGAG
14,154 core
(SEQ ID NO: 38)
TTGTTAAAAAATCCAACCGTGAACGTTAATGCGGGGGGTGAGTGCAAT
CGATACCTCATAGAAT
14,218 core
(SEQ ID NO: 39)
ACCCTTCGTGACGCTCTCGGCTGTTTTTGATATTTTGGCGGAGGAAAT
GCCGTGAATTCGTCTG
15,59 core
(SEQ ID NO: 40)
GATCCATTTCCGAGCGCTTTCCCGACGCGCGCCACGTTCTTCGGGGTC
AAACCTATAAAAGAAC
15,123 core
(SEQ ID NO: 41)
TAGATAAATATGGTGTGAAGATGACTATTTGACAACCTCCACATAAGT
ATTGTTCTCCAGTACC
15,187 core
(SEQ ID NO: 42)
CACTCCGACAATCAGTATAGGAACTCGATGACCTGTCCAAGGAAGGCG
CATATCCAGTAACATC
16,90 core
(SEQ ID NO: 43)
CAAAAACCCCCCCTGATGCCGACGGGCGGCATCTCTCGAAGCGCTATA
CGCTGTCATAAGTTCC
16,154 core
(SEQ ID NO: 44)
TGATGTAAATGGTATATTGCTATCTTAGGGCCGGGAGCTCGACGTGGT
CATTCAAATAAGTCGA
16,218 core
(SEQ ID NO: 45)
GCCAACATATTCTAATCGAGCCGACTGGCGACCGCTGCCAACGCTCAT
CGTGTTGCGTCAGGAT
17,59 core
(SEQ ID NO: 46)
CGCTATCTCCATGGCTTTGTCCTATTGTGTACTAGTCCACATGGTCCG
CGTTGCATCTCACAGT
17,123 core
(SEQ ID NO: 47)
TTCGTGACTAATCGATATTCCGGCCCAGCGTACGGGATAAGAACCAAC
AATGTGGCCCAACGGT
17,187 core
(SEQ ID NO: 48)
CCTCGCCAGCCAGCAAAGAGCCCGGAAAGAAGTCTCATTTTAAGCATC
TATGCTTCAGAATATC
18,90 core
(SEQ ID NO: 49)
CACAACACAGCGGCATGACGGCATGCTCAGACAAGTCATTCACAAATT
GAAGGCGATCCATAGC
18,154 core
(SEQ ID NO: 50)
ACATCGTTGCTGACTACTATAGGTCAGACGAATAAGAACGGCTCCCCC
CGAGAAGATTGGTACG
18,218 core
(SEQ ID NO: 51)
GTTGGGCAATTACTAGACTTCGGGTCAAGGGTCTTAGGAGACGCTATT
TCCATTTGAATTGTAA
19,59 core
(SEQ ID NO: 52)
ATGAAATATTCTTTCAAAACCACGAAACCATCAACGTCAGGCACAACT
GTCAGAACCTGAGGAA
19,123 core
(SEQ ID NO: 53)
GTGGAAGGTGCCCATGTGCCCACATTGGAACGTCTGCATTGGCCCATG
TCTCAAGCTCAGCCCC
19,187 core
(SEQ ID NO: 54)
AGGTGAAGATATCTGGACCCAATCGGGTAATCGGCTAATGAGTCACCC
GCGCTTTTGTCACTCG
3,7 boundary
(SEQ ID NO: 55)
TTTTGGGCCTCCGTAGTGTGATACGAATCACAATACTCGCATCCATGC
CATCACCAAGGCGCCACTGGTTTT
5,7 boundary
(SEQ ID NO: 56)
TTTTCATTGGGGGCAGGTTATCGTCAGCGTGAGATTCTCACACAAATC
CCATAAACCATTATGAGATGTTTT
7,7 boundary
(SEQ ID NO: 57)
TTTTCGTGACACTATGAGAATGAACGTTTATGAAAAGTACTAACCACG
AGGCTTCGCTCCGACCTCTATTTT
9,7 boundary
(SEQ ID NO: 58)
TTTTGATCCAATGTTAGACAGGATTTCCCTCCAACCATTGAGCCATCC
AGTCGGAGAGCCAGGGCAAGTTTT
11,7 boundary
(SEQ ID NO: 59)
TTTTGGTTAGACCCAGTGTCGGAAATCTAAGTTGTCATCTTCAGTTGG
AAGAGATCAGGCGTTCGATCTTTT
13,7 boundary
(SEQ ID NO: 60)
TTTTTCAGAGCGTCGCCCAAATTTATGGCGCTCGCGGATATGTGTCGT
AGCCACCACGCTGATTCCATTTTT
15,7 boundary
(SEQ ID NO: 61)
TTTTTCACACAGTCCAATCGGAACCGTGAACCCTCAGCATATTACGAT
AAAGATTTATGTCCCTGCTATTTT
17,7 boundary
(SEQ ID NO: 62)
TTTTCAGAGGGCAAATCCAGGCGTGTCTGAATAAGGAACGATACTACA
ACTTTAGGGGACCTCGGCAATTTT
19,7 boundary
(SEQ ID NO: 63)
TTTTAGCAACCTATCTGTCTGTCTATAAAGGCTCAAGAGGTGCGGGGT
GCTTACGCCATCAGCGCCAGTTTT
2,270 boundary
(SEQ ID NO: 64)
TTTTAATATTAACAGCGCCGACATCTTCTTTCACCTTAAATGTAATTC
GTACAACAGCTCACGTCAATTTTT
4,270 boundary
(SEQ ID NO: 65)
TTTTTGAGAGACCCCCAGATGAAAATGTTATCTCTTGCTACCCTGTGC
TTAACAGTATACCTCATGGCTTTT
6,270 boundary
(SEQ ID NO: 66)
TTTTAATGCCCTTGGAGTGCATCGATTTGGAGTATTAAAAACAGTCGT
GAAACGACTCAGGCGCCAAGTTTT
8,270 boundary
(SEQ ID NO: 67)
TTTTGTCTATACTGGGGGCGACGATGATGGTTAAATCTCGGAAACATT
CATATGGGGAGTTAGGCTCCTTTT
10,270 boundary
(SEQ ID NO: 68)
TTTTGCTCGAGTAATAGTGTCATAACGCGTAAAGATCTAACGCCAGAA
TACGGTCTACGTACAGAGACTTTT
12,270 boundary
(SEQ ID NO: 69)
TTTTTGGAAGGAATATGGCCCAAGACCAGTCGAGACCACCCGAACGGC
CCTCATCCACGATTTTCACTTTTT
14,270 boundary
(SEQ ID NO: 70)
TTTTTCCCGTGAGGGGCCGAAACGCAACTAGCGGGGGCACGAAAACAG
CACAACCGCCTTGGGTTCGATTTT
16,270 boundary
(SEQ ID NO: 71)
TTTTGCTGCGTAAACGGAAACATCGCTACGCGGGAACCCCTCATATAC
TCTTCGATGGCGCGCACCCCTTTT
18,270 boundary
(SEQ ID NO: 72)
TTTTCTCCCTACCTATTCGGTACGGCGGATGATGCGGATCTTTTTCCA
AGCCGCAGTAAAAATGCGATTTTT
1,7 boundary
(SEQ ID NO: 73)
TTTTCGCCGTAAGCGCTGGAGGCGGTGTGGGTGACG
1,43 boundary
(SEQ ID NO: 74)
ATTATAATGTCAATTCACTTCCTAGAATGCGGGATACGGGCCTCACAT
CCGTAGTCGAAGTTAA
1,107 boundary
(SEQ ID NO: 75)
TCAGTAGACGAACTCCTTTGGGCGGAGGACAGGAACGTGAAGCACGAT
CAACCGCCCTCATCTG
1,171 boundary
(SEQ ID NO: 76)
GTCAGTTCGTAGTCACTGCCCACATTAGGCTCTCCGACATCCATACCT
GTGTGCTTGTGCGAAC
1,235 boundary
(SEQ ID NO: 77)
ACTCCTAAGCTACCCAAAGAAATGATAAATGGTTTT
20,42 boundary
(SEQ ID NO: 78)
TTATACAGGGGTAGTCGGATACCTCGAAGGCCTTTT
20,106 boundary
(SEQ ID NO: 79)
GTTCGGCATTGCACCCAACACAGTGTTGGACCGTGCCGACATGAACTG
TCGAACACGTTGCCCC
20,170 boundary
(SEQ ID NO: 80)
GTTTAATAGTTGTGTAGAGCTGGACGTGGAACAAGACCTGGCGCTGCT
CAGACGTCACATGTAG
20,234 boundary
(SEQ ID NO: 81)
GTTTCTCGGCCCATCAACGACAGCTCCCAGTAGGGGCTTGTGTCTGAT
TCACGGTAGTACCACG
20,270 boundary
(SEQ ID NO: 82)
TTTTGCCAATACGTACCATCTAACTTAAAAGTCAAT
1,7 red trigger
(SEQ ID NO: 83)
TTTTCGCCGTAAGCGCTGGAATTATAATGTCAATTCACTTCCTAGAAT
GCGG
1,59 red trigger
(SEQ ID NO: 84)
GAGGGTCCGGAACTAAGAAGGAGGTGATCTCGTCAGTAGACGAACTCC
TTTGGGCGGAGGACAG
1,123 red trigger
(SEQ ID NO: 85)
GTTCTTGAACCCGTGTATCCATACTCTCGGACGTCAGTTCGTAGTCAC
TGCCCACATTAGGCTC
1,187 red trigger
(SEQ ID NO: 86)
TACGTCCGGCGGCGATGTCCAACCCGTGGTGGAAGAAATGATAAATGG
AATATTAACAGCGCCG
20,90 red trigger
(SEQ ID NO: 87)
TTATACAGGGGTAGTCACGACAGCTCCCAGTAAGCAACCTATCTGTCT
TCTCATTTTAAGCATC
20,154 red trigger
(SEQ ID NO: 88)
GTTCGGCATTGCACCCATGAAATATTCTTTCAAAACCACGAAACCATC
TCGAACACGTTGCCCC
20,218 red trigger
(SEQ ID NO: 89)
GTTTAATAGTTGTGTAGTGGAAGGTGCCCATGTGCCCACATTGGAACG
CAGACGTCACATGTAG
20,270 red trigger
(SEQ ID NO: 90)
TTTTAGGTGAAGATATCTGGACCCAATCGGGTAATCTCACGGTAGTAC
CACG
4,26 green trigger
(SEQ ID NO: 91)
GTGTGCTTGTGCGAACACTCCTAAGCTACCCAGAGTCATATGGCCCTG
ACCAAGGCGCCACTGG
6,26 green trigger
(SEQ ID NO: 92)
TACCCGCCACAGGGCCTAAATGTAATTCGTACGTGCACATCAGAGATG
GGGCCTCCGTAGTGTG
8,26 green trigger
(SEQ ID NO: 93)
ACTCTCTCTATGGAGGGCTACCCTGTGCTTAATGCGACAAGGTACACG
CATTGGGGGCAGGTTA
10,26 green trigger
(SEQ ID NO: 94)
CTTTTAAAAGCAGTTTAAAAACAGTCGTGAAAGATTACCGTACGGTTT
CGTGACACTATGAGAA
12,26 green trigger
(SEQ ID NO: 95)
AGACCTGAGAACGCGCCTCGGAAACATTCATACGTTGCTGTCGGCCGT
GATCCAATGTTAGACA
14,26 green trigger
(SEQ ID NO: 96)
AAGTGAATTTATGTTCCTAACGCCAGAATACGGTAACAGATGTCACAG
GGTTAGACCCAGTGTC
16,26 green trigger
(SEQ ID NO: 97)
AAGAGATAAGTTCAACCACCCGAACGGCCCTCGATCCATTTCCGAGCG
TCAGAGCGTCGCCCAA
18,26 green trigger
(SEQ ID NO: 98)
CTGTCCAAGGAAGGCGGCACGAAAACAGCACACGCTATCTCCATGGCT
TCACACAGTCCAATCG
19,11 green trigger
(SEQ ID NO: 99)
CCCCTCATATACTCTTGTGCCGACATGAACTGCAGAGGGCAAATCCAG
2,266 green trigger
(SEQ ID NO: 100)
CAACCGCCCTCATCTGGTGAACAAAGTGCAAGTGAGAGACCCCCAGAT
3,251 green trigger
(SEQ ID NO: 101)
CGGGACCCCATGTCAGCACTTGAACTTAAAGTAGCATTAAACAGGTTA
AATGCCCTTGGAGTGC
5,251 green trigger
(SEQ ID NO: 102)
GAGCTGATTAGAAATATTTCCATTGGACGGCCTAGGGGTGTGGAGTGC
GTCTATACTGGGGGCG
7,251 green trigger
(SEQ ID NO: 103)
CAACATTTATTCGTGTGGCAGCAGCACTGCCACACCTTACACCGTCAA
GCTCGAGTAATAGTGT
9,251 green trigger
(SEQ ID NO: 104)
CAATTACTTGTGTAGTATCGGTTGAGGGGAAGGAACATTTGATCCGGG
TGGAAGGAATATGGCC
11,251 green trigger
(SEQ ID NO: 105)
AACACGGAGTTCCTGTGTGGTGCTCTTCACTTTCATGGCACTTAAGGC
TCCCGTGAGGGGCCGA
13,251 green trigger
(SEQ ID NO: 106)
TAGAATTCCTTCCTGTCTTCCGTTGAAAACTTACCCTTCGTGACGCTC
GCTGCGTAAACGGAAA
15,251 green trigger
(SEQ ID NO: 107)
CAACCTCCACATAAGTGCCGTGAATTCGTCTGGCCAACATATTCTAAT
CTCCCTACCTATTCGG
17,251 green trigger
(SEQ ID NO: 108)
CGGGATAAGAACCAACCGTGTTGCGTCAGGATGTTGGGCAATTACTAG
TCCATTTGAATTGTAA
Sequences of the 11 x 7 52-bp DNA-origami relay
array (Scaffold p7560).
1,40 core
(SEQ ID NO: 109)
AACGCCATCAAAAGTATAAGCAAATATTTAAATTGTAAATCAGAGCAT
AAAG
1,72 core
(SEQ ID NO: 110)
ACATTAAATGTGAAATCATATGTACCCCGGTTGATAATCCTTTTGCGG
GAGA
1,104 core
(SEQ ID NO: 111)
GCGGATTGACCGTGTCTGGAGCAAACAAGAGAATCGATGCTCATATAT
TTTA
1,136 core
(SEQ ID NO: 112)
ACCGTGCATCTGCAGGGTAGCTATTTTTGAGAGATCTACGGGTGAGAA
AGGC
2,55 core
(SEQ ID NO: 113)
AAACAGGAAGATTATAATTCGCGTCTGGCCTTCCTGTAGTGCTGCAAG
GCGA
2,87 core
(SEQ ID NO: 114)
AAACTAGCATGTCGCGAGTAACAACCCGTCGGATTCTCCGGCGATCGG
TGCG
2,119 core
(SEQ ID NO: 115)
TCATTGCCTGAGAAATGGGATAGGTCACGTTGGTGTAGAGGAAACCAG
GCAA
3,40 core
(SEQ ID NO: 116)
CTAAATCGGTTGTCTACTAATAGTAGTAGCATTAACATCTCAGAAGCA
AAGC
3,72 core
(SEQ ID NO: 117)
AGCCTTTATTTCACTGTTTAGCTATATTTTCATTTGGGGTCAAATATC
GCGT
3,104 core
(SEQ ID NO: 118)
AATGCAATGCCTGAACGAGTAGATTTAGTTTGACCATTACTCCAACAG
GTCA
3,136 core
(SEQ ID NO: 119)
CGGAGACAGTCAAGTACGGTGTCTGGAAGTTTCATTCCAGTCATTTTT
GCGG
4,55 core
(SEQ ID NO: 120)
GGTGGCATCAATTACCAAAAACATTATGACCCTGTAATAAGAAAAGCC
CCAA
4,87 core
(SEQ ID NO: 121)
AATGGTCAATAACACGCAAGGATAAAAATTTTTAGAACCAACGGTAAT
CGTA
4,119 core
(SEQ ID NO: 122)
TCCCAATTCTGCGAGTAATGTGTAGGTAAAGATTCAAAAAAAGGCTAT
CAGG
5,40 core
(SEQ ID NO: 123)
GGATTGCATCAAACCCTCAAATGCTTTAAACAGTTCAGATTGGGCTTG
AGAT
5,72 core
(SEQ ID NO: 124)
TTTAATTCGAGCTAGACTGGATAGCGTCCAATACTGCGGCGATTTTAA
GAAC
5,104 core
(SEQ ID NO: 125)
GGATTAGAGAGTAGAGAGGCTTTTGCAAAAGAAGTTTTGTACGTTAAT
AAAA
5,136 core
(SEQ ID NO: 126)
ATGGCTTAGAGCTGCAACACTATCATAACCCTCGTTTACATCAGTTGA
GATT
6,55 core
(SEQ ID NO: 127)
ATTCATTGAATCCAAGATTAAGAGGAAGCCCGAAAGACTCGCGAGCTG
AAAA
6,87 core
(SEQ ID NO: 128)
TAGTAAAATGTTTTCAAAGCGAACCAGACCGGAAGCAAAGATACATTT
CGCA
6,119 core
(SEQ ID NO: 129)
AAACCAAAATAGCCCTTTAATTGCTCCTTTTGATAAGAGTATAACAGT
TGAT
7,40 core
(SEQ ID NO: 130)
GGTTTAATTTCAAACCCAAATCAACGTAACAAAGCTGCTGGGTAGCAA
CGGC
7,72 core
(SEQ ID NO: 131)
TGGCTCATTATACAGGCTGGCTGACCTTCATCAAGAGTAGTTTCCATT
AAAC
7,104 core
(SEQ ID NO: 132)
CGAACTAACGGAAAACTGACCAACTTTGAAAGAGGACAGAAACGAAAG
AGGC
7,136 core
(SEQ ID NO: 133)
TAGGAATACCACATCCATGTTACTTAGCCGGAACGAGGCATTATACCA
AGCG
8,55 core
(SEQ ID NO: 134)
CCGGATATTCATTCTTTAATCATTGTGAATTACCTTATGAATCGTCAT
AAAT
8,87 core
(SEQ ID NO: 135)
AGACCAGGCGCATCAGTCAGGACGTTGGGAAGAAAAATCCCAGAGGGG
GTAA
8,119 core
(SEQ ID NO: 136)
CATAAGGGAACCGCAACATTATTACAGGTAGAAAGATTCCAGACGACG
ATAA
9,40 core
(SEQ ID NO: 137)
TACAGAGGCTTTGTCGCTGAGGCTTGCAGGGAGTTAAAGCCCAATAGG
AACC
9,72 core
(SEQ ID NO: 138)
GGGTAAAATACGTGTTGCGCCGACAATGACAACAACCATACAACGCCT
GTAG
9,104 core
(SEQ ID NO: 139)
AAAAGAATACACTGGTTTATCAGCTTGCTTTCGAGGTGAAAGTTTTGT
CGTC
9,136 core
(SEQ ID NO: 140)
CGAAACAAAGTACCACGTTGAAAATCTCCAAAAAAAAGGTGCTAAACA
ACTT
10,55 core
(SEQ ID NO: 141)
CCGATATATTCGGAGGACTAAAGACTTTTTCATGAGGAAATCTTGACA
AGAA
10,87 core
(SEQ ID NO: 142)
CTTGATACCGATAAATGCCACTACGAAGGCACCAACCTAATGAACGGT
GTAC
10,119 core
(SEQ ID NO: 143)
CTTTAATTGTATCAAAACACTCATCTTTGACCCCCAGCGGCAGACGGT
CAAT
11,40 core
(SEQ ID NO: 144)
CATGTACCGTAACAGTACCGCCACCCTCAGAACCGCCACAGCATTGAC
AGGA
11,72 core
(SEQ ID NO: 145)
CATTCCACAGACAGGTTGATATAAGTATAGCCCGGAATAACAAATAAA
TCCT
11,104 core
(SEQ ID NO: 146)
TTTCCAGACGTTATTAGGATTAGCGGGGTTTTGCTCAGTCGTTCCAGT
AAGC
11,136 core
(SEQ ID NO: 147)
TCAACAGTTTCAGATTATTCTGAAACATGAAAGTATTAAATAAGTTTT
AACG
12,55 core
(SEQ ID NO: 148)
ACTCAGGAGGTTTACTGAGTTTCGTCACCAGTACAAACTCGCCCACGC
ATAA
12,87 core
(SEQ ID NO: 149)
GTGCCGTCGAGAGGCCCTCATAGTTAGCGTAACGATCTAATTTCTTAA
ACAG
12,119 core
(SEQ ID NO: 150)
CTCAAGAGAAGGAGTAAATGAATTTTCTGTATGGGATTTCTCCAAAAG
GAGC
13,40 core
(SEQ ID NO: 151)
GGTTGAGGCAGGTGCCGCCACCCTCAGAACCGCCACCCTTTTGTCACA
ATCA
13,72 core
(SEQ ID NO: 152)
CATTAAAGCCAGACATAATCAAAATCACCGGAACCAGAGGGGCGACAT
TCAA
13,104 core
(SEQ ID NO: 153)
GTCATACATGGCTTTTTCATCGGCATTTTCGGTCATAGCTCATTAAAG
GTGA
13,136 core
(SEQ ID NO: 154)
GGGTCAGTGCCTTGTAATCAGTAGCGACAGAATCAAGTTGCCAGCAAA
ATCA
14,55 core
(SEQ ID NO: 155)
CGCCTCCCTCAGACAGACGATTGGCCTTGATATTCACAAGGTGTATCA
CCGT
14,87 core
(SEQ ID NO: 156)
TTTGCCATCTTTTATGGAAAGCGCAGTCTCTGAATTTACACCAGGCGG
ATAA
14,119 core
(SEQ ID NO: 157)
AGACTGTAGCGCGTTTGATGATACAGGAGTGTACTGGTAGAGGCTGAG
ACTC
15,40 core
(SEQ ID NO: 158)
ATAGAAAATTCATGTTAGCAAACGTAGAAAATACATACAGCCAGTTAC
AAAA
15,72 core
(SEQ ID NO: 159)
CCGATTGAGGGAGAACGGAATACCCAAAAGAACTGGCATGATTTTTTG
TTTA
15,104 core
(SEQ ID NO: 160)
ATTATCACCGTCAAAGCAGATAGCCGAACAAAGTTACCAACATAAAAA
CAGG
15,136 core
(SEQ ID NO: 161)
CCAGTAGCACCATGAAACAATGAAATAGCAATAGCTATCCAAAGTCAG
AGGG
16,55 core
(SEQ ID NO: 162)
TATTACGCAGTATATGGTTTACCAGCGCCAAAGACAAAACCACCACCG
GAAC
16,87 core
(SEQ ID NO: 163)
GAAACGCAATAATGGAAGGTAAATATTGACGGAAATTATCCCCTTATT
AGCG
16,119 core
(SEQ ID NO: 164)
TTTTAAGAAAAGTCCGACTTGAGCCATTTGGGAATTAGATGCCTTTAG
CGTC
17,40 core
(SEQ ID NO: 165)
TAAACAGCCATATAGTTGCTATTTTGCACCCAGCTACAAAATAAGAGA
ATAT
17,72 core
(SEQ ID NO: 166)
ACGTCAAAAATGATTTTAGCGAACCTCCCGACTTGCGGGCGACAATAA
ACAA
17,104 core
(SEQ ID NO: 167)
GAAGCGCATTAGAGCAAGCAAATCAGATATAGAAGGCTTTAGATAAGT
CCTG
17,136 core
(SEQ ID NO: 168)
TAATTGAGCGCTAAACAAGCAAGCCGTTTTTATTTTCATTAGAAACCA
ATCA
18,55 core
(SEQ ID NO: 169)
TTAAATCAAGATTTATTTATCCCAATCCAAATAAGAAACGATTAAGAC
TCCT
18,87 core
(SEQ ID NO: 170)
AGAACGCGAGGCGAAATAGCAGCCTTTACAGAGAGAATAGAAGGAAAC
CGAG
18,119 core
(SEQ ID NO: 171)
ACCGCGCCCAATACGGGAGAATTAACTGAACACCCTGAATTACCGAAG
CCCT
19,40 core
(SEQ ID NO: 172)
AAAGTACCGACAAAACAGTAGGGCTTAATTGAGAATCGCCTGTAAATC
GTCG
19,72 core
(SEQ ID NO: 173)
CATGTTCAGCTAAGTTTAGTATCATATGCGTTATACAAACGATAGCTT
AGAT
19,104 core
(SEQ ID NO: 174)
AACAAGAAAAATATAAGGCGTTAAATAAGAATAAACACCCATAGGTCT
GAGA
19,136 core
(SEQ ID NO: 175)
ATAATCGGCTGTCTTTCATCTTCTGACCTAAATTTAATGACTATATGT
AAAT
20,55 core
(SEQ ID NO: 176)
AAAGCCAACGCTCAAGGTAAAGTAATTCTGTCCAGACGAAGGTTTTGA
AGCC
20,87 core
(SEQ ID NO: 177)
CTAGAAAAAGCCTTGCAGAACGCGCCTGTTTATCAACAAATCCGGTAT
TCTA
20,119 core
(SEQ ID NO: 178)
ACCGTGTGATAAAATATCCCATCCTAATTTACGAGCATGCGTAGGAAT
CATT
21,40 core
(SEQ ID NO: 179)
CTATTAATTAATTTTTAACAATTTCATTTGAATTACCTTTGGCCCTGA
GAGA
21,72 core
(SEQ ID NO: 180)
TAAGACGCTGAGACTGAGCAAAAGAAGATGATGAAACAAGAAAATCCT
GTTT
21,104 core
(SEQ ID NO: 181)
GACTACCTTTTTATTGAATACCAAGTTACAAAATCGCGCAAAGAATAG
CCCG
21,136 core
(SEQ ID NO: 182)
GCTGATGCAAATCGTAACAGTACCTTTTACATCGGGAGACACTATTAA
AGAA
22,55 core
(SEQ ID NO: 183)
AAAATTAATTACATTCCCTTAGAATCCTTGAAAACATAGTTCTTACCA
GTAT
22,87 core
(SEQ ID NO: 184)
TCATTTCAATTACAGAGTCAATAGTGAATTTATCAAAATGGAATCATA
ATTA
22,119 core
(SEQ ID NO: 185)
TCGCCTGATTGCTACCTCCGGCTTAGGTTGGGTTATATAGTTTGAAAT
ACCG
2,23 Green trigger
(SEQ ID NO: 186)
TTAAAATTCGCATTAAATTTTTGTTAAATCAGCTCATTTTGTAAAACG
ACGG
4,23 Green trigger
(SEQ ID NO: 187)
AGGCAAGGCAAAGAATTAGCAAAATTAAGCAATAAAGCCCGTTAATAT
TTTG
6,23 Green trigger
(SEQ ID NO: 188)
CATAAATCAAAAATCAGGTCTTTACCCTGACTATTATAGCAATAAATC
ATAC
8,23 Green trigger
(SEQ ID NO: 189)
AGGCTTGCCCTGACGAGAAACACCAGAACGAGTAGTAAAAAACGAGAA
TGAC
10,23 Green trigger
(SEQ ID NO: 190)
GATCGTCACCCTCAGCAGCGAAAGACAGCATCGGAACGACATTCAGTG
AATA
12,23 Green trigger
(SEQ ID NO: 191)
ACCCTCAGAGCCACCACCCTCATTTTCAGGGATAGCAAGGCCGCTTTT
GCGG
14,23 Green trigger
(SEQ ID NO: 192)
CTCAGAGCCGCCACCAGAACCACCACCAGAGCCGCCGCCCCTCAGAAC
CGCC
16,23 Green trigger
(SEQ ID NO: 193)
ATATAAAAGAAACGCAAAGACACCACGGAATAAGTTTATCAGAGCCAC
CACC
18,23 Green trigger
(SEQ ID NO: 194)
CTTACCAACGCTAACGAGCGTCTTTCCAGAGCCTAATTTTAAAGGTGG
CAAC
20,23 Green trigger
(SEQ ID NO: 195)
GCCAACATGTAATTTAGGCAGAGGCATTTTCGAGCCAGTTTTTATCCT
GAAT
22,23 Green trigger
(SEQ ID NO: 196)
AGTACATAAATCAATATATGTGAGTGAATAACCTTGCTTCATATTTAA
CAAC
2,151 Green trigger
(SEQ ID NO: 197)
ATTAATGCCGGAGCAGTTTGAGGGGACGACGACAGTATCGGCCTCAGG
AAGA
4,151 Green trigger
(SEQ ID NO: 198)
ATATGCAACTAAAATCACCATCAATATGATATTCAACCGTTCTAGCTG
ATAA
6,151 Green trigger
(SEQ ID NO: 199)
AGGCATAGTAAGATAATTGCTGAATATAATGCTGTAGCTCAACATGTT
TTAA
8,151 Green trigger
(SEQ ID NO: 200)
ATCCGCGACCTGCTTCAACTAATGCAGATACATAACGCCAAAAGGAAT
TACG
10,151 Green trigger
(SEQ ID NO: 201)
ATAATAATTTTTTAACGGAGATTTGTATCATCGCCTGATAAATTGTGT
CGAA
12,151 Green trigger
(SEQ ID NO: 202)
TATTTCGGAACCTCGGAGTGAGAATAGAAAGGAACAACTAAAGGAATT
GCGA
14,151 Green trigger
(SEQ ID NO: 203)
CGATAGCAGCACCGAGTAACAGTGCCCGTATAAACAGTTAATGCCCCC
TGCC
16,151 Green trigger
(SEQ ID NO: 204)
ATAATAAGAGCAATACCATTAGCAAGGCCGGAAACGTCACCAATGAAA
CCAT
18,151 Green trigger
(SEQ ID NO: 205)
CGCACTCATCGAGATATCAGAGAGATAACCCACAAGAATTGAGTTAAG
CCCA
20,151 Green trigger
(SEQ ID NO: 206)
ATATTTTAGTTAATTTCCTTATCATTCCAAGAACGGGTATTAAACCAA
GTAC
22,151 Green trigger
(SEQ ID NO: 207)
AGATGAATATACACAATCGCAAGACAAAGAACGCGAGAAAACTTTTTC
AAAT
23,40 Red Trigger
(SEQ ID NO: 208)
GTTGCAGCAAGCGGTCCACGCTGGTTTGCCCCAGCAGGCACATCAAGA
AAAC
23,72 Red Trigger
(SEQ ID NO: 209)
GATGGTGGTTCCGAAATCGGCAAAATCCCTTATAAATCAAGAGGCGAA
TTAT
23,104 Red Trigger
(SEQ ID NO: 210)
AGATAGGGTTGAGTGTTGTTCCAGTTTGGAACAAGAGTCAACAATAAC
GGAT
0,23 Red Trigger
(SEQ ID NO: 211)
CCAGTGCCAAGCTTTCTCAGGAGAAGCCAGGGTGGATGTTCTTCTAAG
TGGT
0,55 Red Trigger
(SEQ ID NO: 212)
TTAAGTTGGGTAACGCCAGGGTTTTCCCAGTCACGACGTTTTAACCAA
TAGG
0,87 Red Trigger
(SEQ ID NO: 213)
GGCCTCTTCGCTATTACGCCAGCTGGCGAAAGGGGGATGCCAGCTTTC
ATCA
0,119 Red Trigger
(SEQ ID NO: 214)
AGCGCCATTCGCCATTCAGGCTGCGCAACTGTTGGGAAGGTGGGAACA
AACG
0,151 Red Trigger
(SEQ ID NO: 215)
TCGCACTCCAGCCAGCTTTCCGGCACCGCTTCTGGTGCCTGGGCGCAT
CGTA
2,23 Green-triggertoehold
(SEQ ID NO: 216)
TTAAAATTCGCATTAAATTTTTGTTAAATCAGCTCATTTTGTAAAACG
ACGGAACTTGACCT
4,23 Green-triggertoehold
(SEQ ID NO: 217)
AGGCAAGGCAAAGAATTAGCAAAATTAAGCAATAAAGCCCGTTAATAT
TTTGTGCTCGTGTT
6,23 Green-triggertoehold
(SEQ ID NO: 218)
CATAAATCAAAAATCAGGTCTTTACCCTGACTATTATAGCAATAAATC
ATACGCACGCCAAA
8,23 Green-triggertoehold
(SEQ ID NO: 219)
AGGCTTGCCCTGACGAGAAACACCAGAACGAGTAGTAAAAAACGAGAA
TGACGAGATGCTTC
10,23 Green-triggertoehold
(SEQ ID NO: 220)
GATCGTCACCCTCAGCAGCGAAAGACAGCATCGGAACGACATTCAGTG
AATATTGTGGAACT
12,23 Green-triggertoehold
(SEQ ID NO: 221)
ACCCTCAGAGCCACCACCCTCATTTTCAGGGATAGCAAGGCCGCTTTT
GCGGCGACAACGCA
14,23 Green-triggertoehold
(SEQ ID NO: 222)
CTCAGAGCCGCCACCAGAACCACCACCAGAGCCGCCGCCCCTCAGAAC
CGCCGGATCTACGT
16,23 Green-triggertoehold
(SEQ ID NO: 223)
ATATAAAAGAAACGCAAAGACACCACGGAATAAGTTTATCAGAGCCAC
CACCCGGAGTTGCT
18,23 Green-triggertoehold
(SEQ ID NO: 224)
CTTACCAACGCTAACGAGCGTCTTTCCAGAGCCTAATTTTAAAGGTGG
CAACGCGACATTGG
20,23 Green-triggertoehold
(SEQ ID NO: 225)
GCCAACATGTAATTTAGGCAGAGGCATTTTCGAGCCAGTTTTTATCCT
GAATGATCTGTCAG
22,23 Green-triggertoehold
(SEQ ID NO: 226)
AGTACATAAATCAATATATGTGAGTGAATAACCTTGCTTCATATTTAA
CAACCGAAAAACAT
2,151 Green-triggertoehold
(SEQ ID NO: 227)
ATTAATGCCGGAGCAGTTTGAGGGGACGACGACAGTATCGGCCTCAGG
AAGACCGTCCCCGA
4,151 Green-triggertoehold
(SEQ ID NO: 228)
ATATGCAACTAAAATCACCATCAATATGATATTCAACCGTTCTAGCTG
ATAAGGCGGACACT
6,151 Green-triggertoehold
(SEQ ID NO: 229)
AGGCATAGTAAGATAATTGCTGAATATAATGCTGTAGCTCAACATGTT
TTAAGATTGACGCG
8,151 Green-triggertoehold
(SEQ ID NO: 230)
ATCCGCGACCTGCTTCAACTAATGCAGATACATAACGCCAAAAGGAAT
TACGGTTTTGTAGA
10,151 Green-triggertoehold
(SEQ ID NO: 231)
ATAATAATTTTTTAACGGAGATTTGTATCATCGCCTGATAAATTGTGT
CGAAAGGTTAGGGG
12,151 Green-triggertoehold
(SEQ ID NO: 232)
TATTTCGGAACCTCGGAGTGAGAATAGAAAGGAACAACTAAAGGAATT
GCGAAATAGGTTAG
14,151 Green-triggertoehold
(SEQ ID NO: 233)
CGATAGCAGCACCGAGTAACAGTGCCCGTATAAACAGTTAATGCCCCC
TGCCATTGAGTGGC
16,151 Green-triggertoehold
(SEQ ID NO: 234)
ATAATAAGAGCAATACCATTAGCAAGGCCGGAAACGTCACCAATGAAA
CCATTTAAGAATGT
18,151 Green-triggertoehold
(SEQ ID NO: 235)
CGCACTCATCGAGATATCAGAGAGATAACCCACAAGAATTGAGTTAAG
CCCAATTATAGTGT
20,151 Green-triggertoehold
(SEQ ID NO: 236)
ATATTTTAGTTAATTTCCTTATCATTCCAAGAACGGGTATTAAACCAA
GTACAGTAATCTCT
22,151 Green-triggertoehold
(SEQ ID NO: 237)
AGATGAATATACACAATCGCAAGACAAAGAACGCGAGAAAACTTTTTC
AAATGATTAACGGT
2,23-r Green-triggerremove
(SEQ ID NO: 238)
AGGTCAAGTTCCGTCGTTTTACAAAATGAGCTGATTTAACAAAAATTT
AATGCGAATTTTAA
4,23-r Green-triggerremove
(SEQ ID NO: 239)
AACACGAGCACAAAATATTAACGGGCTTTATTGCTTAATTTTGCTAAT
TCTTTGCCTTGCCT
6,23-r Green-triggerremove
(SEQ ID NO: 240)
TTTGGCGTGCGTATGATTTATTGCTATAATAGTCAGGGTAAAGACCTG
ATTTTTGATTTATG
8,23-r Green-triggerremove
(SEQ ID NO: 241)
GAAGCATCTCGTCATTCTCGTTTTTTACTACTCGTTCTGGTGTTTCTC
GTCAGGGCAAGCCT
10,23-r Green-triggerremove
(SEQ ID NO: 242)
AGTTCCACAATATTCACTGAATGTCGTTCCGATGCTGTCTTTCGCTGC
TGAGGGTGACGATC
12,23-r Green-triggerremove
(SEQ ID NO: 243)
TGCGTTGTCGCCGCAAAAGCGGCCTTGCTATCCCTGAAAATGAGGGTG
GTGGCTCTGAGGGT
14,23-r Green-triggerremove
(SEQ ID NO: 244)
ACGTAGATCCGGCGGTTCTGAGGGGCGGCGGCTCTGGTGGTGGTTCTG
GTGGCGGCTCTGAG
16,23-r Green-triggerremove
(SEQ ID NO: 245)
AGCAACTCCGGGTGGTGGCTCTGATAAACTTATTCCGTGGTGTCTTTG
CGTTTCTTTTATAT
18,23-r Green-triggerremove
(SEQ ID NO: 246)
CCAATGTCGCGTTGCCACCTTTAAAATTAGGCTCTGGAAAGACGCTCG
TTAGCGTTGGTAAG
20,23-r Green-triggerremove
(SEQ ID NO: 247)
CTGACAGATCATTCAGGATAAAAACTGGCTCGAAAATGCCTCTGCCTA
AATTACATGTTGGC
22,23-r Green-triggerremove
(SEQ ID NO: 248)
ATGTTTTTCGGTTGTTAAATATGAAGCAAGGTTATTCACTCACATATA
TTGATTTATGTACT
21,51-r Green-triggerremove
(SEQ ID NO: 249)
TCGGGGACGGTCTTCCTGAGGCCGATACTGTCGTCGTCCCCTCAAACT
GCTCCGGCATTAAT
41,51-r Green-triggerremove
(SEQ ID NO: 250)
AGTGTCCGCCTTATCAGCTAGAACGGTTGAATATCATATTGATGGTGA
TTTTAGTTGCATAT
6,151-r Green-triggerremove
(SEQ ID NO: 251)
CGCGTCAATCTTAAAACATGTTGAGCTACAGCATTATATTCAGCAATT
ATCTTACTATGCCT
8,151-r Green-triggerremove
(SEQ ID NO: 252)
TCTACAAAACCGTAATTCCTTTTGGCGTTATGTATCTGCATTAGTTGA
AGCAGGTCGCGGAT
10,151-r Green-triggerremove
(SEQ ID NO: 253)
CCCCTAACCTTTCGACACAATTTATCAGGCGATGATACAAATCTCCGT
TAAAAAATTATTAT
12,151-r Green-triggerremove
(SEQ ID NO: 254)
CTAACCTATTTCGCAATTCCTTTAGTTGTTCCTTTCTATTCTCACTCC
GAGGTTCCGAAATA
14,151-r Green-triggerremove
(SEQ ID NO: 255)
GCCACTCAATGGCAGGGGGCATTAACTGTTTATACGGGCACTGTTACT
CGGTGCTGCTATCG
16,151-r Green-triggerremove
(SEQ ID NO: 256)
ACATTCTTAAATGGTTTCATTGGTGACGTTTCCGGCCTTGCTAATGGT
ATTGCTCTTATTAT
18,151-r Green-triggerremove
(SEQ ID NO: 257)
ACACTATAATTGGGCTTAACTCAATTCTTGTGGGTTATCTCTCTGATA
TCTCGATGAGTGCG
20,151-r Green-triggerremove
(SEQ ID NO: 258)
AGAGATTACTGTACTTGGTTTAATACCCGTTCTTGGAATGATAAGGAA
ATTAACTAAAATAT
22,151-r Green-triggerremove
(SEQ ID NO: 259)
ACCGTTAATCATTTGAAAAAGTTTTCTCGCGTTCTTTGTCTTGCGATT
GTGTATATTCATCT
23,40 Red-triggertoehold
(SEQ ID NO: 260)
GTTGCAGCAAGCGGTCCACGCTGGTTTGCCCCAGCAGGCACATCAAGA
AAACCTCCCCGGGT
23,72 Red-triggertoehold
(SEQ ID NO: 261)
GATGGTGGTTCCGAAATCGGCAAAATCCCTTATAAATCAAGAGGCGAA
TTATGTGGCTCCTT
23,104 Red-triggertoehold
(SEQ ID NO: 262)
AGATAGGGTTGAGTGTTGTTCCAGTTTGGAACAAGAGTCAACAATAAC
GGATCATCTGACAA
23,136 Red-triggertoehold
(SEQ ID NO: 263)
CGTGGACTCCAACGTCAAAGGGCGAAAAACCGTCTATCACAGGTTTAA
CGTCCTATCGCCAT
0,55 Red-triggertoehold
(SEQ ID NO: 264)
TTAAGTTGGGTAACGCCAGGGTTTTCCCAGTCACGACGTTTTAACCAA
TAGGCGATTGTTTC
0,87 Red-triggertoehold
(SEQ ID NO: 265)
GGCCTCTTCGCTATTACGCCAGCTGGCGAAAGGGGGATGCCAGCTTTC
ATCATGCGGACGGT
0,119 Red-triggertoehold
(SEQ ID NO: 266)
AGCGCCATTCGCCATTCAGGCTGCGCAACTGTTGGGAAGGTGGGAACA
AACGGTTGTCCTCA
0,151 Red-triggertoehold
(SEQ ID NO: 267)
TCGCACTCCAGCCAGCTTTCCGGCACCGCTTCTGGTGCCTGGGCGCAT
CGTATAGTTTGGGC
23,40-r Red-triggerremove
(SEQ ID NO: 268)
ACCCGGGGAGGTTTTCTTGATGTGCCTGCTGGGGCAAACCAGCGTGGA
CCGCTTGCTGCAAC
23,72-r Red-triggerremove
(SEQ ID NO: 269)
AAGGAGCCACATAATTCGCCTCTTGATTTATAAGGGATTTTGCCGATT
TCGGAACCACCATC
23,104-r Red-triggerremove
(SEQ ID NO: 270)
TTGTCAGATGATCCGTTATTGTTGACTCTTGTTCCAAACTGGAACAAC
ACTCAACCCTATCT
23,136-r Red-triggerremove
(SEQ ID NO: 271)
ATGGCGATAGGACGTTAAACCTGTGATAGACGGTTTTTCGCCCTTTGA
CGTTGGAGTCCACG
0,55-r Red-triggerremove
(SEQ ID NO: 272)
GAAACAATCGCCTATTGGTTAAAACGTCGTGACTGGGAAAACCCTGGC
GTTACCCAACTTAA
0,87-r Red-triggerremove
(SEQ ID NO: 273)
ACCGTCCGCATGATGAAAGCTGGCATCCCCCTTTCGCCAGCTGGCGTA
ATAGCGAAGAGGCC
0,119-r Red-triggerremove
(SEQ ID NO: 274)
TGAGGACAACCGTTTGTTCCCACCTTCCCAACAGTTGCGCAGCCTGAA
TGGCGAATGGCGCT
0,151-r Red-triggerremove
(SEQ ID NO: 275)
GCCCAAACTATACGATGCGCCCAGGCACCAGAAGCGGTGCCGGAAAGC
TGGCTGGAGTGCGA
0,55 RO52-Tube_linker
(SEQ ID NO: 276)
TTAAGTTGGGTAAGTCCACGCTGGTTTGCCCCAGCAGGCACATCAAGA
AAAC
0,87 RO52-Tube_linker
(SEQ ID NO: 277)
GGCCTCTTCGCTAAAATCGGCAAAATCCCTTATAAATCAAGAGGCGAA
TTAT
0,119 RO52-Tube_linker
(SEQ ID NO: 278)
AGCGCCATTCGCCTGTTGTTCCAGTTTGGAACAAGAGTCAACAATAAC
GGAT
23,40 RO52-Tube_linker
(SEQ ID NO: 279)
GTTGCAGCAAGCGCGCCAGGGTTTTCCCAGTCACGACGTTTTAACCAA
TAGG
23,72 RO52-Tube_linker
(SEQ ID NO: 280)
GATGGTGGTTCCGTTACGCCAGCTGGCGAAAGGGGGATGCCAGCTTTC
ATCA
23,104 RO52-Tube_linker
(SEQ ID NO: 281)
AGATAGGGTTGAGATTCAGGCTGCGCAACTGTTGGGAAGGTGGGAACA
AACG
23,136 RO52-Tube_linker
(SEQ ID NO: 282)
CGTGGACTCCAACAGCTTTCCGGCACCGCTTCTGGTGCCTGGGCGCAT
CGTA
Sequences of the 3D 42-bp DNA-brick relay array.
0,0
(SEQ ID NO: 283)
TTTACCCACGACAGTTTACGATGGCATGGATGTCCTCGATCCTGTCAA
ACTTATTT
0,42
(SEQ ID NO: 284)
TTTTCCTTCCGAAGCGTGCAAATCCGTCCGAGTCTCACATCCTCACGG
CCCTATTT
0,84
(SEQ ID NO: 285)
TTTAACCTACTCTATCCACCACGGGTTGGACCGGGTACGAACATACCG
TAGTCTTCAGGGTTTT
0,49
(SEQ ID NO: 286)
TTTCGGAAGGAGGTGATCTCGTCGCATCCATGGATCGGAGTTGACAGG
CTTAACACTTT
0,91
(SEQ ID NO: 287)
TTTAGTAGGTTGCTAATCCATACTCTCGGACGCTTAAGAGCCGTGAGG
TTCCCGTATTT
0,125
(SEQ ID NO: 288)
TTTATACATCCAAACTGGTCCAACGTGATAGACGGTATGTTAGGCTCC
TTT
1,0
(SEQ ID NO: 289)
TTTCCTTGGGAGGGCTTCATCATGCCATCGTAAACTGTCGTGGGTTTT
1,7
(SEQ ID NO: 290)
TTTTCCCAAGGAGCTAATACTCCAAATCGATTGATCATGGCAAAGGGC
ATTTAGGCCCTGTTTT
1,49
(SEQ ID NO: 291)
TTTGGACACGAACGCTGACGAAAAAAATGGAGGCTTCAATAACCGCCC
ATCACTAATCCGATTT
1,91
(SEQ ID NO: 292)
TTTCATACATGCACGCACACTATTGTCTTTTCGATGTAGCAAATCCCA
CGACTGGATGGCTCAATTCGCTTT
2,49
(SEQ ID NO: 293)
TTTGTCAGCGTGAGATTCCGTACTCCATGATCCTGGTTCCGCCTAAAT
GTAATTCGTTT
2,91
(SEQ ID NO: 294)
TTTTGTGCGTGGCTTACGCGCTGAATTGAAGCTCGTAGTCTTAGTGAT
GGCGGGTATTT
2,125
(SEQ ID NO: 295)
TTTCTTGCGAGGGTCCGCTACATCGCACTTCGTCCAGTCGCACCTGTG
TTT
3,0
(SEQ ID NO: 296)
TTTCTAGTCCACATCGCGGCGCATAATCGATTTGGAGTATTAGCTTTT
3,7
(SEQ ID NO: 297)
TTTTGGACTAGGTGAACCGAGTTGACTTTCTGGCCAACTGCCACACAC
AGGGGACCTGTGTTTT
3,49
(SEQ ID NO: 298)
TTTCGCGAATGCTTAATCCTTCTGGTCGTAATTTGTCCAAATTTGCAC
TGCAGGCCGCCGCTTT
3,91
(SEQ ID NO: 299)
TTTACTAGGTAATTTAGATTTGCGTCCCGGTTGCGTCTCTGCAAGTGC
GCGGCTTTGGTAATACTCTACTTT
4,49
(SEQ ID NO: 300)
TTTGGATTAAGGTGGCTGAATGTGCAGTTGGCAAATTCACGGTCCCCT
CATGAATTTTT
4,91
(SEQ ID NO: 301)
TTTATCTAAATTGATAAGCGAGTATTGGACAAATGAGCAGGGCCTGCA
TCTCGGATTTT
4,125
(SEQ ID NO: 302)
TTTGGGGATTGACGCAAGAGACGCTATATATGAAAGCCGCGACAATTA
TTT
5,0
(SEQ ID NO: 303)
TTTACACCTCACGAATGGCTCAAGCAGAAAGTCAACTCGGTTCACTTT
5,7
(SEQ ID NO: 304)
TTTTGAGGTGTACGAGGGCACGAAGCGGTAACCGAAGCCCCTGCACTC
CAGAAAGTGGTTATTT
5,49
(SEQ ID NO: 305)
TTTTCTCGAGGACAGGTGAAAGAAGATGTGAATTGACATTGGGTTACA
TTGACAGAGAGATTTT
5,91
(SEQ ID NO: 306)
TTTCCGTCGCGACACCGCCGGAGTTCGTCTACTGATTAACGGTACGTA
GCGTTGTAGCACCGTTGAGCATTT
6,49
(SEQ ID NO: 307)
TTTTCACCTGTGAACAAAGTGCAAGGGCTTCGGATTCGTAACTTTCTG
TGGAAGAGTATCACAGTGCGATTT
6,91
(SEQ ID NO: 308)
TTTGGCGGTGTGGGTGACGATTATAATGTCAACTTTAAGTTCTGTCAA
CTGCAATTGCGTCAGCGGTTGTTT
6,125
(SEQ ID NO: 309)
TTTGACCGTAGTCGAAGTTAATCAATTAAAAATACAACGCCCCTATTT
ACGGCTTAACTGATTT
7,0
(SEQ ID NO: 310)
TTTAAGACATAAAGCAAGTATTGTGTTACCGCTTCGTGCCCTCGTTTT
7,41
(SEQ ID NO: 311)
TTTTATCGCGATACTATACGAATCACAATACTTGCTTTATGTCTTTTT
7,83
(SEQ ID NO: 312)
TTTTGTCAGCACTTGAACTTAAAGTACGTGCGACGTCTTT
7,125
(SEQ ID NO: 313)
TTTTCTGAGAACGGTTTTTTTAATAAGGCCCAACCCGTTT
8,0
(SEQ ID NO: 314)
TTTCACACTCATGTACCCATTT
8,42
(SEQ ID NO: 315)
TTTGCCTGCAAGAGTTCCTGTGTGGTGTTT
8,84
(SEQ ID NO: 316)
TTTAGTGGAAACAGTAAGTGAATTTATTTT
8,54
(SEQ ID NO: 317)
TTTTCCACTTGCAGGCCAACACTTTGAGACGACGAGATCACCTTT
8,96
(SEQ ID NO: 318)
TTTTCACTTTTCCACTCCTCCTAATTCTAAGTATGGATTAGCTTT
8,125
(SEQ ID NO: 319)
TTTCCCATAGACCGAGAGTTTGGATGTATTTT
9,0
(SEQ ID NO: 320)
TTTATGGTGCGCTTGAGCTGGAAAAATGGGTATTT
9,34
(SEQ ID NO: 321)
TTTGCTAGTCATATGGAGTAGGCGACCAAACCGCTATTTATTT
9,76
(SEQ ID NO: 322)
TTTCTAGGATGAGATTACTGTCATTTCCGACTCGGGGCAATTT
9,12
(SEQ ID NO: 323)
TTTCGTGACGCACCATGCACGAACATTTTTCCTTT
9,54
(SEQ ID NO: 324)
TTTACGGTACTCCATAGACTACGAAGCGGTTTTGGCATGCTTT
9,96
(SEQ ID NO: 325)
TTTGAACGAGTAATCTGAGTGTTCCCGAGTCGAATGGAGGTTT
10,34
(SEQ ID NO: 326)
TTTTAGTCTGTTGACTAGCTAAGTTCCCCACGCCGCCTCGTGTCCAAC
GGTGGCACCTGATTTGCACGCTTTTT
10,76
(SEQ ID NO: 327)
TTTTAGCTTAGCATCCTAGTAGGGATAACATTTTCAGCATGTATGGAA
AGCAAAGGTTCCGTGGTGGATAGTTT
10,118
(SEQ ID NO: 328)
TTTAGTCTAACACCCTGAAGACTATAAGCTGTGGGGATTT
10,54
(SEQ ID NO: 329)
TTTTCAGTTCGTAGTCACAGACTACATTTAGTACGGAATCTCGGCGGC
GTGGGGACTCCGATCCATGATGAAGCCCTTT
10,96
(SEQ ID NO: 330)
TTTTGTGCGAACACTCCTAAGCTACCCACTCAGCGCGTAAGCCTGAAA
ATGTTATCTCTTAAGAGGTGCCACCGTTTTT
10,125
(SEQ ID NO: 331)
TTTGTTAGACTCACACGGACCCTCGCAAGTCCCCACAGCTTATCTATC
ACAACCTTTGCTTTCTTT
11,0
(SEQ ID NO: 332)
TTTCTACCGAGGGAGGTGCACCTCGAATCTCATTT
11,34
(SEQ ID NO: 333)
TTTGGCGGGTAGCCTCGTGATAAGCCTTATCTTGCACTGCTTT
11,76
(SEQ ID NO: 334)
TTTCGATTTAGAGCTTGAAGGTATAAGGAATAAATGTTGTTTT
11,12
(SEQ ID NO: 335)
TTTTACGCCTCGGTAGTGTCCCTGTTCGAGGTTTT
11,54
(SEQ ID NO: 336)
TTTCTAACCACGAGGCGCCTACCCGCAAGATACATACTTTTTT
11,96
(SEQ ID NO: 337)
TTTGCACATTCAAGCTGCCTGGACATTTATTCGTGTGGCATTT
12,34
(SEQ ID NO: 338)
TTTTAGACACGTACCCGCCACAGGGCGGTGACCGCGGCATTCGCGACC
TATGCCAAAACTCCATTTTTTTCTTT
12,76
(SEQ ID NO: 339)
TTTGTACGACACTAAATCGTCGGATATGCCCGGAGTGTACCTAGTGTA
ATAGCGGCTTGAAAAGACAATAGTTT
12,118
(SEQ ID NO: 340)
TTTCGGTCTTTGCGAATTGAGCCATGCTAAACCGCAATTT
12,54
(SEQ ID NO: 341)
TTTACCACGGGTAGGCCGTGTCTAACTTACACATTCAGCCACCCGCGG
TCACCGCGGAACCAGATGCGCCGCGATGTTT
12,96
(SEQ ID NO: 342)
TTTGACAAGTCCAGGCTGTCGTACGTACCTACTCGCTTATCACACTCC
GGGCATAGACTACGATTTTGGCATAGGTTTT
12,125
(SEQ ID NO: 343)
TTTAAAGACCGTTGTTTGCGTCAATCCCCTTGCGGTTTAGCACGAAGT
GCAAGCCGCTATTACTTT
13,0
(SEQ ID NO: 344)
TTTGTATACTGGGAAGACTTTGTCTTTATCGTTTT
13,34
(SEQ ID NO: 345)
TTTCGATACTTCTCTACGGCTCGGAAATTATAGGTTTATTTTT
13,76
(SEQ ID NO: 346)
TTTTATTCTTTACTATTTATTGTTTCAATTTCCTCCGCCATTT
3,12
(SEQ ID NO: 347)
TTTAGGTTCAGTATACCCCGGAGGAAAGACAATTT 1
13,54
(SEQ ID NO: 348)
TTTTACACCCGTAGAGGGTCTAAAACCTATAAAAGAACCTTTT
13,96
(SEQ ID NO: 349)
TTTTAAGGTAAATAGTGACGGATTGAGGAAATATTGTTCTTTT
14,34
(SEQ ID NO: 350)
TTTTCTCTGGGAAGTATCGACACAGTGTAGTCTCTACCCTCGAGATCG
CTACCGGGGAATTACGACCAGAATTT
14,76
(SEQ ID NO: 351)
TTTCAATTATAAAAGAATAGCGGCGTCCATCAGAGCCCGCGACGGTTA
CTCATCGGCCAACCGGGACGCAATTT
14,118
(SEQ ID NO: 352)
TTTGGTCCCGTGTAGAGTATTACCGTACTTCATTGTGTTT
14,54
(SEQ ID NO: 353)
TTTCTCGCTTTAGACCCCCAGAGAGTTAATTGCACTTTGTTCGTAGAG
ACTACACGTGAATTTCTTGAGCCATTCGTTT
14,96
(SEQ ID NO: 354)
TTTCATCGAATCCGTCTATAATTGGACGGATAATCGTCACCCGGCTCT
GATGGACCTGCTCATTCCCCGGTAGCGATTT
14,125
(SEQ ID NO: 355)
TTTACGGGACCGAGAGTTCGACTACGGTCCACAATGAAGTACCATATA
TAGGCCGATGAGTAATTT
15,0
(SEQ ID NO: 356)
TTTTCGCACTGTCGCATGTTTT
15,34
(SEQ ID NO: 357)
TTTCAAGATTCCAACCGCTCTTGGTTCTTT
15,76
(SEQ ID NO: 358)
TTTACAGAGGATCAGTTAATCTGTGCTTTT
15,41
(SEQ ID NO: 359)
TTTGAATCTTGTAACCTAGTATCGCGATAGCGTAAACGACGTCGCACG
TATTCACATCTTCTTTTT
15,83
(SEQ ID NO: 360)
TTTTCCTCTGTATCTCTCAAGTGCTGACATGGGGTCCCGGGTTGGGCC
TTGTAGACGAACTCCTTT
15,125
(SEQ ID NO: 361)
TTTTGCTCAACGGTGCAACCGTTCTCAGATTT
16,0
(SEQ ID NO: 362)
TTTTGGGTACACCATGCCCACAGCCTTAAGTGGTGGTACAAACGCAAG
GATCTTTACGCGTTATGAACCTTT
16,42
(SEQ ID NO: 363)
TTTAGGAACTCCGTGTTCGTATTCTGGCGTTAGATCGCAGGCAGGGAT
TCCCTTGTGACGACGAGATTCTTT
16,84
(SEQ ID NO: 364)
TTTACTTACTGTTCACCACCAGTTACTAATTGCACGCTACTCTTGAGG
CGCTGTGTTTTTCTTT
16,33
(SEQ ID NO: 365)
TTTCTCTTCACTTAAGATAGCCAGCTTGCGTTAGCAACGTAGACTAAT
TGAATGACAACTTTTT
16,75
(SEQ ID NO: 366)
TTTGTTCCTAACGCCAATGTGGCAATCCCTGCTCGGCTGCATGGTGGT
TCATATGCGTTCCTTT
16,125
(SEQ ID NO: 367)
TTTACCGTTCCCAGGGCAATTAGTCCACAAGACCTCAAGACCAGTCGA
GACATGAATCTCGACAGTGGATTT
17,0
(SEQ ID NO: 368)
TTTAGCTCAAGGCGGATAACGGGTGCTGTGGGCATGGGTATACCGGGA
CAATCGAGGATCTCAAAGTGTTGTTT
17,55
(SEQ ID NO: 369)
TTTAGCGTGGAGAATACGAACACGGTGGACGAGCTAGATGTGAGATAG
AATTAGGAGGTTT
17,97
(SEQ ID NO: 370)
TTTTCCATGTGAACTGGTGGTGAAAGTGAGGAGGGCATCGTACCCCTC
GGTCTATGGGTTT
17,33
(SEQ ID NO: 371)
TTTAAGACCTGGCTATACCCGTTATCCGCTCACGGTGTTAAGTGTCCC
GGTATACTGAGTGTGTTT
17,75
(SEQ ID NO: 372)
TTTAGAACTGCCACATTCCACGCTAGGATACCGTTACGGGAACTAGCT
CGTTT
17,117
(SEQ ID NO: 373)
TTTCCCAGTCTTGTGGCACATGGAACAGCCGTTCGGAGCCTATGCCCT
CCTTT
18,13
(SEQ ID NO: 374)
TTTGTAATCGCCTGATAACATCCAGTATGGCGGCGGAGGATGCGCCCC
TGCGATAGTTT
18,55
(SEQ ID NO: 375)
TTTGACATCGCTGGTTTCAGCTCGCGCAGAGAGGCCTATCGGTGGCAG
ATCGTTGCTTT
18,97
(SEQ ID NO: 376)
TTTAAGTTACGACCATCGGGTCATACGGGGAGGAGACAGAATCTACTC
TTT
18,33
(SEQ ID NO: 377)
TTTCATTAGTTATCAGCCCGTGGGCGCCGCCAGAGACGGGTATGTGAT
GGTTTGTGCTGAGTTT
18,75
(SEQ ID NO: 378)
TTTGTCGATGAAACCAAGATGCATGCCTCTCTATCCCGACTTCAACTA
TCGCACAAAGCATTTT
18,125
(SEQ ID NO: 379)
TTTACCCTTACCACCCCCGATGGTATAGAGAGCTCCTCCCTTTAATGC
CTACCGCAACGATAGTACCGTTTT
19,0
(SEQ ID NO: 380)
TTTGCACCTCCATTCCGACGGGGGGCGATTACTACCCCTCTGTTCTAC
AAGCCCTTTGAAATGTTT
19,55
(SEQ ID NO: 381)
TTTCATAATCCGCGATGTCTAAATACTGACCAGATGAGGGCGGTTGTG
GGTTT
19,97
(SEQ ID NO: 382)
TTTGGTATAGTCGTAACTTTTGCCGCACAAGCACACATGGGATTTGTG
TGTTT
19,33
(SEQ ID NO: 383)
TTTGAATTCCCACGGGCCCCCGTCGGAATGCGTACGAATTACTTGTAG
AACAGAGGTTCGTGCTTT
19,75
(SEQ ID NO: 384)
TTTGAGACATGCATCTGGATTATGCGGATGTTAGTACCCGCCTCATCT
GGTTT
19,117
(SEQ ID NO: 385)
TTTTGAATCTCTCTATACTATACCGCTCCTGTGCCACAGGTGTGTGTG
CTTTT
20,13
(SEQ ID NO: 386)
TTTACGTAGGGAATTCTACGTTTACAACTGGGAAGCACTGCGAGAAAA
GCTAGCCCTTT
20,55
(SEQ ID NO: 387)
TTTTTGGTAGGATGGCAGTTGACAACACAGTGTCAGGGATGCTGGCCA
ATAAGTCATTT
20,97
(SEQ ID NO: 388)
TTTTTCACGACTGTTTTTACAAGTACTACTTGCAATTACGCACACAAT
TTT
20,33
(SEQ ID NO: 389)
TTTAGGGAGTAGAATTGGTTTCAGCTTCCCAGATAACCGCAAGAAGTT
GCCGGGCCCCACGTTT
20,75
(SEQ ID NO: 390)
TTTGCAGCACTGCCATTCTGGCTATGACACTGTAGACAATGAATGGGG
CTAGCAACAGGGTTTT
20,125
(SEQ ID NO: 391)
TTTTTTAAAAGCAGTTTAAAAACAGGTCTCGTTTGCAAGTGCCTTCGT
CCAGATGACTTATTTTCAAGTTTT
21,0
(SEQ ID NO: 392)
TTTAGTCTTCCAACTGAAGATAACCCCTACGTTGAGAGCGTCTGTCAA
AGGTGTGTGGTAAGTTTT
21,55
(SEQ ID NO: 393)
TTTTTCTCAGGCCTACCAAGCAGTGTGGTACTTTCCAGTGCAAATGGT
ACTTT
21,97
(SEQ ID NO: 394)
TTTTGAGGCCGGTCGTGAAACAACTTGTCGATAGGTGGCACTTGCAAC
AATTT
21,33
(SEQ ID NO: 395)
TTTGGACACTGAAACCGTTATCTTCAGTTAACCTAATTCATGCTTTGA
CAGACGCCAGGGACATTT
21,75
(SEQ ID NO: 396)
TTTTGTCGTAGCCAGACCTGAGAACGCGCGTGTAATCCGAGATGGAAA
GTTTT
21,117
(SEQ ID NO: 397)
TTTGCGAGACGAGACCCGGCCTCACCTACCCTTATAATTGTCCACCTA
TCTTT
22,13
(SEQ ID NO: 398)
TTTAATATGCTTGTAACCCACGACGTTCTCTTAAGTTGTAGTATCGTT
TATAGCGCTTT
22,55
(SEQ ID NO: 399)
TTTGCACTCACCCCCCGGTGAGCGGGTTGTTTTCGTGCCGCCTTCCTT
GGACAGAATTT
22,97
(SEQ ID NO: 400)
TTTAAATAATCCTTATAAGCCTTCCTGCAATTTGTGAATGACTTTTCC
TTT
22,33
(SEQ ID NO: 401)
TTTGACCAGGGTTACACGCACACTCTTAAGAGATAAGTTCAACCACCC
GAACGTTT
22,75
(SEQ ID NO: 402)
TTTCCAGTACCGGGGGATAACAGTCGAAAACAGCACACTCTCGAAGCG
CTATATTT
22,125
(SEQ ID NO: 403)
TTTGGCAATTGTTCGCCTTATAAGGAGTCCGCACAAATTGAGGTGCGG
GGTGCTTCTGTCCTTT
23,0
(SEQ ID NO: 404)
TTTACATGCGAAGGTCTTGTCGTAAGCATATTACGATCATCACACTTG
TCGAGTGCAGTTAACTTT
23,55
(SEQ ID NO: 405)
TTTACACGAGTGTGAGTGCAATAAGCGAGGCTGGAGATGTAACCCCCG
TCTTT
23,97
(SEQ ID NO: 406)
TTTGCGGAGCTGATTATTTTGGCGCGATGACCCTCTCTACGTACCCTC
TCTTT
23,41
(SEQ ID NO: 407)
TTTGCGCCAAGACCTAAGTGTGCGTACGACAAGACCTTGATACTCTTC
CAGACAAGTGTGATGCCTCCGGGTTT
23,83
(SEQ ID NO: 408)
TTTTTAAAAGCTTCTCACTGTTATACTCGTGTATTCTGACGCAATTGC
AGTCTCCAGCTTT
23,125
(SEQ ID NO: 409)
TTTCATATCTGGAACCGCGGACTCAGCTCCGCCTATTGCCGTAAATAG
GGGAGAGGGTTTT
24,0
(SEQ ID NO: 410)
TTTACCGGGTTCTTGTTGTGGAGGTGTACCACGAGTGATGTCCCGTTT
24,50
(SEQ ID NO: 411)
TTTTGCTATGAGATACCTGCGATCTGATGCTCTGTCTTTT
24,92
(SEQ ID NO: 412)
TTTCCATGGAAATTGAGTAGCGTGGGGTTTCATGCATTTT
24,125
(SEQ ID NO: 413)
TTTATGCATGAAACCCCCCTGGGAACGGTTTT
25,0
(SEQ ID NO: 414)
TTTATTCAATTAGTCTACGTTGCTCCTCCACAACAAGAACCCGGTTTT
25,50
(SEQ ID NO: 415)
TTTACCATGCAGCCGAGTATCTCATAGCAGCCGAGGACGGGACATCAC
TCAAGAGCACCACACTTT
25,92
(SEQ ID NO: 416)
TTTATGTCTCGACTGGTCAATTTCCATGGGCGGCCTTAGACAGAGCAT
CAGGAACATAAATTCTTT
26,8
(SEQ ID NO: 417)
TTTAGATTTCCGATGTTACTGGATATGCCCCGCTAGTACGCGTAAAGA
TCGTCTTCTGGCTGTGGTCGCCTATCCTTTT
26,50
(SEQ ID NO: 418)
TTTTACGTACCGTTACGCGCGAGCGCCATAAATCGACTCGTCACAAGG
GAGTTCTGGAGTGGTGAAATGACGCTGTTTT
26,92
(SEQ ID NO: 419)
TTTATAATGGTATCTCCGTATGACTGTGGGATCAACCGAAAAACACAG
CGCTGGGGCAGTAGATTT
26,125
(SEQ ID NO: 420)
TTTGGTTGATCCCACAGGGTGGTAAGGGTTCTACTGCTTT
27,0
(SEQ ID NO: 421)
TTTAAACCATCACATACCCGTCTCACATCGGAAATCTAAGTTGTCTTT
27,50
(SEQ ID NO: 422)
TTTTTGAAGTCGGGATGTAACGGTACGTAGGAACGCAACTAGCGGGGC
ATTAATGGCATGCCAACAGCCAGTTT
27,92
(SEQ ID NO: 423)
TTTGGTAGGCATTAAAGAGATACCATTATTCCACTGTGTCGATTTATG
GCTCGACCCTCCATTACCACTCCTTT
28,8
(SEQ ID NO: 424)
TTTGAGTTACATTCTCTTGTAAACTAGGCCCTGTAGGGGGGCGCATCC
TCAATTCCTCGGGGCAGGCTTATATCCGTTT
28,50
(SEQ ID NO: 425)
TTTGACTATACCTGTTTGTTGTCAGCTGGTGTGCATGCTGCCACCGAT
AGGTCTCTGACCTTCCTTATACCGGAGCTTT
28,92
(SEQ ID NO: 426)
TTTGAATTCCGTGCTGAGTACTTGAAAGCAACGACAGGAGTAGATTCT
GTATTCAAGATGATTTTT
28,125
(SEQ ID NO: 427)
TTTCTGTCGTTGCTTTAACTGCTTTTAAAAATCATCTTTT
29,0
(SEQ ID NO: 428)
TTTCCGGCAACTTCTTGCGGTTATGAGAATGTAACTCCTCAGCACTTT
29,50
(SEQ ID NO: 429)
TTTCATTCATTGTCTAAACAGGTATAGTCATGCTTTGCCTACAGGGCC
TATCCCTAAAGTATGGCCCCGAGTTT
29,92
(SEQ ID NO: 430)
TTTTCTGGACGAAGGCCAGCACGGAATTCACGGTACTCATGCACACCA
GCGCTGCTGCCACACGAAGGTCATTT
30,8
(SEQ ID NO: 431)
TTTCGCCACCCCTTCCAACGTCGTGTTCTTAGACGCCTTTTCTCGCAG
TGTGTCCTATGAATGTTTCCGAGGCGCGTTT
30,50
(SEQ ID NO: 432)
TTTTACGTGGGGGTATACCCGCTCATACGAAGGTTGGTGGCCAGCATC
CCCGACACATATCTGTGAAACAAGTAGGTTT
30,92
(SEQ ID NO: 433)
TTTTTTCAACGGAAGACAGGAAGGAATTCTAGAGGGCATTGTGTGCGT
AACTCGCGTGCACCCTTT
30,125
(SEQ ID NO: 434)
TTTGCCCTCTAGAATTGCGAACAATTGCCGGGTGCACTTT
31,0
(SEQ ID NO: 435)
TTTCGTTCGGGTGGTTGAACTTATGGAAGGGGTGGCGCGTGGGGCTTT
31,50
(SEQ ID NO: 436)
TTTTTCGAGAGTGTGCATACCCCCACGTAACCCTGTTGGCGTCTAAGA
ACTGGTCAGGTTCTTCATTCATATTT
31,92
(SEQ ID NO: 437)
TTTGCACCCCGCACCTTCTTCCGTTGAAAACTTGAAACCAACCTTCGT
ATACTGGAGAACAATCAGATATGTTT
31,41
(SEQ ID NO: 438)
TTTAACGATACTACAATAGGTCTTGGCGCGAACCAAGAGAATTTT
31,83
(SEQ ID NO: 439)
TTTAAGGAAGGCGGCAGAGAAGCTTTTAAAGCACAGAAATAGTTT
31,125
(SEQ ID NO: 440)
TTTGGAAAAGTCATTCGGTTCCAGATATGTTT

1. A double helix array of nucleic acids comprising intersections of four double stranded arms,
wherein a first double stranded arm and a second double stranded arm share a first polynucleotide strand,
wherein the second double stranded arm and a third double stranded arm share a second polynucleotide strand,
wherein the third double stranded arm and a fourth double stranded arm share a third polynucleotide strand, and
wherein the fourth double stranded arm and the first double strand arm share a fourth polynucleotide strand; and
wherein the first double stranded arm and the second double stranded arm form a first ring, and the third double stranded arm and the fourth double stranded arm form a second ring.

2. The double helix array of claim 1, wherein the second ring comprises a segment that is single stranded.

3. The double helix array of claim 2, wherein the second ring is on the boundary of the array.

4. The double helix array of claim 1, wherein the second double stranded arm and the third double stranded arm form a third ring, and the first double stranded arm and the fourth double stranded arm form a fourth ring.

5. The double helix array of claim 4, wherein the boundary of the array comprises terminal 5′ single stranded polynucleotide sequence and/or a terminal 3′ single stranded polynucleotide sequence comprising segments of poly-T.

6. The double helix array of claim 1 comprising four or more intersections.

7. The double helix array of claim 1, wherein the first polynucleotide strand substantially hybridizes with the array to form a double helix, and optionally comprises a terminal 5′ single stranded polynucleotide segment and/or a terminal 3′ single stranded polynucleotide segment that does not hybridize with the array.

8. The double helix array of claim 4, wherein the first polynucleotide strand substantially hybridizes with the array to form a double helix, and comprises a terminal 5′ single stranded polynucleotide segment that does not hybridize with the array and a terminal 3′ single stranded polynucleotide segment that does not hybridize with the array, and further comprises a fifth polynucleotide segment wherein the fifth polynucleotide segment hybridizes with the terminal 5′ single stranded polynucleotide segment and hybridizes with the terminal 3′ single stranded polynucleotide segment of the first polynucleotide strand.

9. The double helix array of claim 1, further linked covalently or through hydrogen bonding to a ligand, receptor, drug, antibody, aptamer, or combinations thereof.

10. A solid substrate linked covalently or through hydrogen bonding to the double helix array of claim 1.

11. The solid substrate of claim 10 made from glass, plastic, metal, stone, ceramic minerals, or combinations thereof.

12. The solid substrate of claim 10, comprising a plurality of particles, zones or wells.

13. The solid substrate of claim 12, wherein with approximately one double helix array per zone or well.

14. An aqueous solution comprising the double helix array of claim 1 and formamide.

15. The aqueous solution of claim 14, heated to above 40 degrees Celsius.

16. A tube structure comprising the double helix array of claim 1.

17. A method of transforming the confirmation of a double helix array comprising mixing the double helix array of claim 2 and an added single stranded nucleic acid which is configure to hybridize with the segment that is single stranded on the second ring under conditions such that the added single stranded nucleic acid bends or straightens the segment that is single stranded altering the conformation of the double helix array.


SEQUENCE LISTING (440 sequences)

SEQ ID NO: 1 - DNA, 64 bp, Artificial
  Synthetic construct
tacggttttagcatcagagggtccggaactaagaaggaggtgatctcgttgctttatgtcttga

SEQ ID NO: 2 - DNA, 64 bp, Artificial
  Synthetic construct
ggatcacattatcgccgttcttgaacccgtgtatccatactctcggacacgctatcgcgatact

SEQ ID NO: 3 - DNA, 64 bp, Artificial
  Synthetic construct
ctcagggacccataattacgtccggcggcgatgtccaacccgtggtggcacttgaacttaaagt

SEQ ID NO: 4 - DNA, 64 bp, Artificial
  Synthetic construct
gagtcatatggccctgtcaactgaaagaccggacgtgcgacgtcgtttggatttgcacgcttcg

SEQ ID NO: 5 - DNA, 64 bp, Artificial
  Synthetic construct
cacaaccggacgttcatggaattaggaagacccgggaccccatgtcagatagagtaggttgcta

SEQ ID NO: 6 - DNA, 64 bp, Artificial
  Synthetic construct
tctatcttaatcctcctgagcgaatagttaaatacccgccacagggccgtgaacaaagtgcaag

SEQ ID NO: 7 - DNA, 64 bp, Artificial
  Synthetic construct
ttggcgttcacttggcttagctcctagagtttgcctcggtaggttagaccgtactccatgatca

SEQ ID NO: 8 - DNA, 64 bp, Artificial
  Synthetic construct
ttgacgtgtaagtcggcctgctcgatctacacgatgaagccctcccaattaataaggcccaacc

SEQ ID NO: 9 - DNA, 64 bp, Artificial
  Synthetic construct
agcattaaacaggttatcaccttttcactcctggacacgaggcggcgttttccattggacggcc

SEQ ID NO: 10 - DNA, 64 bp, Artificial
  Synthetic construct
gtgcacatcagagatgctgggagagatgcatgtctgagaacggtttttggatacatccaaactg

SEQ ID NO: 11 - DNA, 64 bp, Artificial
  Synthetic construct
cccgtagcatggggagacgcagagaacgcagtgagctgattagaaataggggactccgatccat

SEQ ID NO: 12 - DNA, 64 bp, Artificial
  Synthetic construct
gcagtgccacaattcgttaccgagttctttaaactctctctatggaggaagaggtgccaccgtt

SEQ ID NO: 13 - DNA, 64 bp, Artificial
  Synthetic construct
atatctgcctaagctaggagctctagcgatggggattagtgatggcggtctttggatccttatt

SEQ ID NO: 14 - DNA, 64 bp, Artificial
  Synthetic construct
tctaaacgcagggccaccagataccctaccacgcacctgtggcacattctagatccctgcggtt

SEQ ID NO: 15 - DNA, 64 bp, Artificial
  Synthetic construct
taggggtgtggagtgcgacagtttgaatcgatatctatcacaacctttggcagcagcactgcca

SEQ ID NO: 16 - DNA, 64 bp, Artificial
  Synthetic construct
tgcgacaaggtacacggttctaaggttggcgacatttaattaaatatgcaagctctaaatcgtc

SEQ ID NO: 17 - DNA, 64 bp, Artificial
  Synthetic construct
atctcctttttacccatgtctctctatgtctccaacatttattcgtgtgctttccatacatgct

SEQ ID NO: 18 - DNA, 64 bp, Artificial
  Synthetic construct
actcttcgaaaatttcgacctcaccactttaccttttaaaagcagtttagcttccccacagctt

SEQ ID NO: 19 - DNA, 64 bp, Artificial
  Synthetic construct
ggtgtctttcaccaatgtgttctgaccccgtcacgccagggacagcgattctaggaaacgtaat

SEQ ID NO: 20 - DNA, 64 bp, Artificial
  Synthetic construct
ctgcgttaacgtcaactcattagcggactattgccgtgtctaacttacgcaaaacgaattgctt

SEQ ID NO: 21 - DNA, 64 bp, Artificial
  Synthetic construct
caccttacaccgtcaagtgtctttatccgcctgtaccatttgcactggatcggttgaggggaag

SEQ ID NO: 22 - DNA, 64 bp, Artificial
  Synthetic construct
gattaccgtacggtttgtaatttccctcctctcgaacattaaaaacttcacacacctttgacag

SEQ ID NO: 23 - DNA, 64 bp, Artificial
  Synthetic construct
gcgaccaaagtagctgactcctcaatgtaagacaattacttgtgtagtaaagtaccacgggtag

SEQ ID NO: 24 - DNA, 64 bp, Artificial
  Synthetic construct
ccgttttcaatgctggacatccgtttcgtactagacctgagaacgcgctaaggggccgattagt

SEQ ID NO: 25 - DNA, 64 bp, Artificial
  Synthetic construct
tctagcaacgccatcctggcgataaatctgtaggacactgaaaccgttgaaaaacacagcgcct

SEQ ID NO: 26 - DNA, 64 bp, Artificial
  Synthetic construct
gtcttagatgagcacgggaggtaagctgagcctactcaatttccatggtgtacccacatcagct

SEQ ID NO: 27 - DNA, 64 bp, Artificial
  Synthetic construct
gaacatttgatccgggtagcccccaccattcctagactaattgaatatgtggtgctcttcactt

SEQ ID NO: 28 - DNA, 64 bp, Artificial
  Synthetic construct
cgttgctgtcggccgtgtatcgaactatcactaaggctgtgggcatgggcggccttagacagag

SEQ ID NO: 29 - DNA, 64 bp, Artificial
  Synthetic construct
cacgtggaggtaaggcagtcgaccacacgtacaacacggagttcctgtgcatgaaaccccacgc

SEQ ID NO: 30 - DNA, 64 bp, Artificial
  Synthetic construct
taaggagccccgtccgacgactcatgaactttaagtgaatttatgttcccttgcgttagcaacg

SEQ ID NO: 31 - DNA, 64 bp, Artificial
  Synthetic construct
cgcgagggcacgccggtgaagaacgccaatagacctacttgtttcacacgtaacggtacgtagg

SEQ ID NO: 32 - DNA, 64 bp, Artificial
  Synthetic construct
gatatggacaaacaactgcccccgggcaccctacaagggtgcacgcgattattccactgtgtcg

SEQ ID NO: 33 - DNA, 64 bp, Artificial
  Synthetic construct
tcatggcacttaaggcaatataggcgtattagcaagggaatccctgctcttccgttgaaaactt

SEQ ID NO: 34 - DNA, 64 bp, Artificial
  Synthetic construct
gtaacagatgtcacagaatcgcccctcgttattcatacggagataccagacgagacccggcctc

SEQ ID NO: 35 - DNA, 64 bp, Artificial
  Synthetic construct
ttcagtagacctgcttgggtaacataacaaattagaattccttcctgtcggctgcatggtggtt

SEQ ID NO: 36 - DNA, 64 bp, Artificial
  Synthetic construct
tcatcaggcggtttcgttttcgatcggggtaaaagagataagttcaacatgaatctcgtcgtca

SEQ ID NO: 37 - DNA, 64 bp, Artificial
  Synthetic construct
agggctgccacgattatccctacgaccggttactgaccagggttacaagccatacatactggag

SEQ ID NO: 38 - DNA, 64 bp, Artificial
  Synthetic construct
ttgttaaaaaatccaaccgtgaacgttaatgcggggggtgagtgcaatcgatacctcatagaat

SEQ ID NO: 39 - DNA, 64 bp, Artificial
  Synthetic construct
acccttcgtgacgctctcggctgtttttgatattttggcggaggaaatgccgtgaattcgtctg

SEQ ID NO: 40 - DNA, 64 bp, Artificial
  Synthetic construct
gatccatttccgagcgctttcccgacgcgcgccacgttcttcggggtcaaacctataaaagaac

SEQ ID NO: 41 - DNA, 64 bp, Artificial
  Synthetic construct
tagataaatatggtgtgaagatgactatttgacaacctccacataagtattgttctccagtacc

SEQ ID NO: 42 - DNA, 64 bp, Artificial
  Synthetic construct
cactccgacaatcagtataggaactcgatgacctgtccaaggaaggcgcatatccagtaacatc

SEQ ID NO: 43 - DNA, 64 bp, Artificial
  Synthetic construct
caaaaaccccccctgatgccgacgggcggcatctctcgaagcgctatacgctgtcataagttcc

SEQ ID NO: 44 - DNA, 64 bp, Artificial
  Synthetic construct
tgatgtaaatggtatattgctatcttagggccgggagctcgacgtggtcattcaaataagtcga

SEQ ID NO: 45 - DNA, 64 bp, Artificial
  Synthetic construct
gccaacatattctaatcgagccgactggcgaccgctgccaacgctcatcgtgttgcgtcaggat

SEQ ID NO: 46 - DNA, 64 bp, Artificial
  Synthetic construct
cgctatctccatggctttgtcctattgtgtactagtccacatggtccgcgttgcatctcacagt

SEQ ID NO: 47 - DNA, 64 bp, Artificial
  Synthetic construct
ttcgtgactaatcgatattccggcccagcgtacgggataagaaccaacaatgtggcccaacggt

SEQ ID NO: 48 - DNA, 64 bp, Artificial
  Synthetic construct
cctcgccagccagcaaagagcccggaaagaagtctcattttaagcatctatgcttcagaatatc

SEQ ID NO: 49 - DNA, 64 bp, Artificial
  Synthetic construct
cacaacacagcggcatgacggcatgctcagacaagtcattcacaaattgaaggcgatccatagc

SEQ ID NO: 50 - DNA, 64 bp, Artificial
  Synthetic construct
acatcgttgctgactactataggtcagacgaataagaacggctccccccgagaagattggtacg

SEQ ID NO: 51 - DNA, 64 bp, Artificial
  Synthetic construct
gttgggcaattactagacttcgggtcaagggtcttaggagacgctatttccatttgaattgtaa

SEQ ID NO: 52 - DNA, 64 bp, Artificial
  Synthetic construct
atgaaatattctttcaaaaccacgaaaccatcaacgtcaggcacaactgtcagaacctgaggaa

SEQ ID NO: 53 - DNA, 64 bp, Artificial
  Synthetic construct
gtggaaggtgcccatgtgcccacattggaacgtctgcattggcccatgtctcaagctcagcccc

SEQ ID NO: 54 - DNA, 64 bp, Artificial
  Synthetic construct
aggtgaagatatctggacccaatcgggtaatcggctaatgagtcacccgcgcttttgtcactcg

SEQ ID NO: 55 - DNA, 72 bp, Artificial
  Synthetic construct
ttttgggcctccgtagtgtgatacgaatcacaatactcgcatccatgccatcaccaaggcgccactggtttt

SEQ ID NO: 56 - DNA, 72 bp, Artificial
  Synthetic construct
ttttcattgggggcaggttatcgtcagcgtgagattctcacacaaatcccataaaccattatgagatgtttt

SEQ ID NO: 57 - DNA, 72 bp, Artificial
  Synthetic construct
ttttcgtgacactatgagaatgaacgtttatgaaaagtactaaccacgaggcttcgctccgacctctatttt

SEQ ID NO: 58 - DNA, 72 bp, Artificial
  Synthetic construct
ttttgatccaatgttagacaggatttccctccaaccattgagccatccagtcggagagccagggcaagtttt

SEQ ID NO: 59 - DNA, 72 bp, Artificial
  Synthetic construct
ttttggttagacccagtgtcggaaatctaagttgtcatcttcagttggaagagatcaggcgttcgatctttt

SEQ ID NO: 60 - DNA, 72 bp, Artificial
  Synthetic construct
tttttcagagcgtcgcccaaatttatggcgctcgcggatatgtgtcgtagccaccacgctgattccattttt

SEQ ID NO: 61 - DNA, 72 bp, Artificial
  Synthetic construct
tttttcacacagtccaatcggaaccgtgaaccctcagcatattacgataaagatttatgtccctgctatttt

SEQ ID NO: 62 - DNA, 72 bp, Artificial
  Synthetic construct
ttttcagagggcaaatccaggcgtgtctgaataaggaacgatactacaactttaggggacctcggcaatttt

SEQ ID NO: 63 - DNA, 72 bp, Artificial
  Synthetic construct
ttttagcaacctatctgtctgtctataaaggctcaagaggtgcggggtgcttacgccatcagcgccagtttt

SEQ ID NO: 64 - DNA, 72 bp, Artificial
  Synthetic construct
ttttaatattaacagcgccgacatcttctttcaccttaaatgtaattcgtacaacagctcacgtcaattttt

SEQ ID NO: 65 - DNA, 72 bp, Artificial
  Synthetic construct
tttttgagagacccccagatgaaaatgttatctcttgctaccctgtgcttaacagtatacctcatggctttt

SEQ ID NO: 66 - DNA, 72 bp, Artificial
  Synthetic construct
ttttaatgcccttggagtgcatcgatttggagtattaaaaacagtcgtgaaacgactcaggcgccaagtttt

SEQ ID NO: 67 - DNA, 72 bp, Artificial
  Synthetic construct
ttttgtctatactgggggcgacgatgatggttaaatctcggaaacattcatatggggagttaggctcctttt

SEQ ID NO: 68 - DNA, 72 bp, Artificial
  Synthetic construct
ttttgctcgagtaatagtgtcataacgcgtaaagatctaacgccagaatacggtctacgtacagagactttt

SEQ ID NO: 69 - DNA, 72 bp, Artificial
  Synthetic construct
tttttggaaggaatatggcccaagaccagtcgagaccacccgaacggccctcatccacgattttcacttttt

SEQ ID NO: 70 - DNA, 72 bp, Artificial
  Synthetic construct
tttttcccgtgaggggccgaaacgcaactagcgggggcacgaaaacagcacaaccgccttgggttcgatttt

SEQ ID NO: 71 - DNA, 72 bp, Artificial
  Synthetic construct
ttttgctgcgtaaacggaaacatcgctacgcgggaacccctcatatactcttcgatggcgcgcacccctttt

SEQ ID NO: 72 - DNA, 72 bp, Artificial
  Synthetic construct
ttttctccctacctattcggtacggcggatgatgcggatctttttccaagccgcagtaaaaatgcgattttt

SEQ ID NO: 73 - DNA, 36 bp, Artificial
  Synthetic construct
ttttcgccgtaagcgctggaggcggtgtgggtgacg

SEQ ID NO: 74 - DNA, 64 bp, Artificial
  Synthetic construct
attataatgtcaattcacttcctagaatgcgggatacgggcctcacatccgtagtcgaagttaa

SEQ ID NO: 75 - DNA, 64 bp, Artificial
  Synthetic construct
tcagtagacgaactcctttgggcggaggacaggaacgtgaagcacgatcaaccgccctcatctg

SEQ ID NO: 76 - DNA, 64 bp, Artificial
  Synthetic construct
gtcagttcgtagtcactgcccacattaggctctccgacatccatacctgtgtgcttgtgcgaac

SEQ ID NO: 77 - DNA, 36 bp, Artificial
  Synthetic construct
actcctaagctacccaaagaaatgataaatggtttt

SEQ ID NO: 78 - DNA, 36 bp, Artificial
  Synthetic construct
ttatacaggggtagtcggatacctcgaaggcctttt

SEQ ID NO: 79 - DNA, 64 bp, Artificial
  Synthetic construct
gttcggcattgcacccaacacagtgttggaccgtgccgacatgaactgtcgaacacgttgcccc

SEQ ID NO: 80 - DNA, 64 bp, Artificial
  Synthetic construct
gtttaatagttgtgtagagctggacgtggaacaagacctggcgctgctcagacgtcacatgtag

SEQ ID NO: 81 - DNA, 64 bp, Artificial
  Synthetic construct
gtttctcggcccatcaacgacagctcccagtaggggcttgtgtctgattcacggtagtaccacg

SEQ ID NO: 82 - DNA, 36 bp, Artificial
  Synthetic construct
ttttgccaatacgtaccatctaacttaaaagtcaat

SEQ ID NO: 83 - DNA, 52 bp, Artificial
  Synthetic construct
ttttcgccgtaagcgctggaattataatgtcaattcacttcctagaatgcgg

SEQ ID NO: 84 - DNA, 64 bp, Artificial
  Synthetic construct
gagggtccggaactaagaaggaggtgatctcgtcagtagacgaactcctttgggcggaggacag

SEQ ID NO: 85 - DNA, 64 bp, Artificial
  Synthetic construct
gttcttgaacccgtgtatccatactctcggacgtcagttcgtagtcactgcccacattaggctc

SEQ ID NO: 86 - DNA, 64 bp, Artificial
  Synthetic construct
tacgtccggcggcgatgtccaacccgtggtggaagaaatgataaatggaatattaacagcgccg

SEQ ID NO: 87 - DNA, 64 bp, Artificial
  Synthetic construct
ttatacaggggtagtcacgacagctcccagtaagcaacctatctgtcttctcattttaagcatc

SEQ ID NO: 88 - DNA, 64 bp, Artificial
  Synthetic construct
gttcggcattgcacccatgaaatattctttcaaaaccacgaaaccatctcgaacacgttgcccc

SEQ ID NO: 89 - DNA, 64 bp, Artificial
  Synthetic construct
gtttaatagttgtgtagtggaaggtgcccatgtgcccacattggaacgcagacgtcacatgtag

SEQ ID NO: 90 - DNA, 52 bp, Artificial
  Synthetic construct
ttttaggtgaagatatctggacccaatcgggtaatctcacggtagtaccacg

SEQ ID NO: 91 - DNA, 64 bp, Artificial
  Synthetic construct
gtgtgcttgtgcgaacactcctaagctacccagagtcatatggccctgaccaaggcgccactgg

SEQ ID NO: 92 - DNA, 64 bp, Artificial
  Synthetic construct
tacccgccacagggcctaaatgtaattcgtacgtgcacatcagagatggggcctccgtagtgtg

SEQ ID NO: 93 - DNA, 64 bp, Artificial
  Synthetic construct
actctctctatggagggctaccctgtgcttaatgcgacaaggtacacgcattgggggcaggtta

SEQ ID NO: 94 - DNA, 64 bp, Artificial
  Synthetic construct
cttttaaaagcagtttaaaaacagtcgtgaaagattaccgtacggtttcgtgacactatgagaa

SEQ ID NO: 95 - DNA, 64 bp, Artificial
  Synthetic construct
agacctgagaacgcgcctcggaaacattcatacgttgctgtcggccgtgatccaatgttagaca

SEQ ID NO: 96 - DNA, 64 bp, Artificial
  Synthetic construct
aagtgaatttatgttcctaacgccagaatacggtaacagatgtcacagggttagacccagtgtc

SEQ ID NO: 97 - DNA, 64 bp, Artificial
  Synthetic construct
aagagataagttcaaccacccgaacggccctcgatccatttccgagcgtcagagcgtcgcccaa

SEQ ID NO: 98 - DNA, 64 bp, Artificial
  Synthetic construct
ctgtccaaggaaggcggcacgaaaacagcacacgctatctccatggcttcacacagtccaatcg

SEQ ID NO: 99 - DNA, 48 bp, Artificial
  Synthetic construct
cccctcatatactcttgtgccgacatgaactgcagagggcaaatccag

SEQ ID NO: 100 - DNA, 48 bp, Artificial
  Synthetic construct
caaccgccctcatctggtgaacaaagtgcaagtgagagacccccagat

SEQ ID NO: 101 - DNA, 64 bp, Artificial
  Synthetic construct
cgggaccccatgtcagcacttgaacttaaagtagcattaaacaggttaaatgcccttggagtgc

SEQ ID NO: 102 - DNA, 64 bp, Artificial
  Synthetic construct
gagctgattagaaatatttccattggacggcctaggggtgtggagtgcgtctatactgggggcg

SEQ ID NO: 103 - DNA, 64 bp, Artificial
  Synthetic construct
caacatttattcgtgtggcagcagcactgccacaccttacaccgtcaagctcgagtaatagtgt

SEQ ID NO: 104 - DNA, 64 bp, Artificial
  Synthetic construct
caattacttgtgtagtatcggttgaggggaaggaacatttgatccgggtggaaggaatatggcc

SEQ ID NO: 105 - DNA, 64 bp, Artificial
  Synthetic construct
aacacggagttcctgtgtggtgctcttcactttcatggcacttaaggctcccgtgaggggccga

SEQ ID NO: 106 - DNA, 64 bp, Artificial
  Synthetic construct
tagaattccttcctgtcttccgttgaaaacttacccttcgtgacgctcgctgcgtaaacggaaa

SEQ ID NO: 107 - DNA, 64 bp, Artificial
  Synthetic construct
caacctccacataagtgccgtgaattcgtctggccaacatattctaatctccctacctattcgg

SEQ ID NO: 108 - DNA, 64 bp, Artificial
  Synthetic construct
cgggataagaaccaaccgtgttgcgtcaggatgttgggcaattactagtccatttgaattgtaa

SEQ ID NO: 109 - DNA, 52 bp, Artificial
  Synthetic construct
aacgccatcaaaagtataagcaaatatttaaattgtaaatcagagcataaag

SEQ ID NO: 110 - DNA, 52 bp, Artificial
  Synthetic construct
acattaaatgtgaaatcatatgtaccccggttgataatccttttgcgggaga

SEQ ID NO: 111 - DNA, 52 bp, Artificial
  Synthetic construct
gcggattgaccgtgtctggagcaaacaagagaatcgatgctcatatatttta

SEQ ID NO: 112 - DNA, 52 bp, Artificial
  Synthetic construct
accgtgcatctgcagggtagctatttttgagagatctacgggtgagaaaggc

SEQ ID NO: 113 - DNA, 52 bp, Artificial
  Synthetic construct
aaacaggaagattataattcgcgtctggccttcctgtagtgctgcaaggcga

SEQ ID NO: 114 - DNA, 52 bp, Artificial
  Synthetic construct
aaactagcatgtcgcgagtaacaacccgtcggattctccggcgatcggtgcg

SEQ ID NO: 115 - DNA, 52 bp, Artificial
  Synthetic construct
tcattgcctgagaaatgggataggtcacgttggtgtagaggaaaccaggcaa

SEQ ID NO: 116 - DNA, 52 bp, Artificial
  Synthetic construct
ctaaatcggttgtctactaatagtagtagcattaacatctcagaagcaaagc

SEQ ID NO: 117 - DNA, 52 bp, Artificial
  Synthetic construct
agcctttatttcactgtttagctatattttcatttggggtcaaatatcgcgt

SEQ ID NO: 118 - DNA, 52 bp, Artificial
  Synthetic construct
aatgcaatgcctgaacgagtagatttagtttgaccattactccaacaggtca

SEQ ID NO: 119 - DNA, 52 bp, Artificial
  Synthetic construct
cggagacagtcaagtacggtgtctggaagtttcattccagtcatttttgcgg

SEQ ID NO: 120 - DNA, 52 bp, Artificial
  Synthetic construct
ggtggcatcaattaccaaaaacattatgaccctgtaataagaaaagccccaa

SEQ ID NO: 121 - DNA, 52 bp, Artificial
  Synthetic construct
aatggtcaataacacgcaaggataaaaatttttagaaccaacggtaatcgta

SEQ ID NO: 122 - DNA, 52 bp, Artificial
  Synthetic construct
tcccaattctgcgagtaatgtgtaggtaaagattcaaaaaaaggctatcagg

SEQ ID NO: 123 - DNA, 52 bp, Artificial
  Synthetic construct
ggattgcatcaaaccctcaaatgctttaaacagttcagattgggcttgagat

SEQ ID NO: 124 - DNA, 52 bp, Artificial
  Synthetic construct
tttaattcgagctagactggatagcgtccaatactgcggcgattttaagaac

SEQ ID NO: 125 - DNA, 52 bp, Artificial
  Synthetic construct
ggattagagagtagagaggcttttgcaaaagaagttttgtacgttaataaaa

SEQ ID NO: 126 - DNA, 52 bp, Artificial
  Synthetic construct
atggcttagagctgcaacactatcataaccctcgtttacatcagttgagatt

SEQ ID NO: 127 - DNA, 52 bp, Artificial
  Synthetic construct
attcattgaatccaagattaagaggaagcccgaaagactcgcgagctgaaaa

SEQ ID NO: 128 - DNA, 52 bp, Artificial
  Synthetic construct
tagtaaaatgttttcaaagcgaaccagaccggaagcaaagatacatttcgca

SEQ ID NO: 129 - DNA, 52 bp, Artificial
  Synthetic construct
aaaccaaaatagccctttaattgctccttttgataagagtataacagttgat

SEQ ID NO: 130 - DNA, 52 bp, Artificial
  Synthetic construct
ggtttaatttcaaacccaaatcaacgtaacaaagctgctgggtagcaacggc

SEQ ID NO: 131 - DNA, 52 bp, Artificial
  Synthetic construct
tggctcattatacaggctggctgaccttcatcaagagtagtttccattaaac

SEQ ID NO: 132 - DNA, 52 bp, Artificial
  Synthetic construct
cgaactaacggaaaactgaccaactttgaaagaggacagaaacgaaagaggc

SEQ ID NO: 133 - DNA, 52 bp, Artificial
  Synthetic construct
taggaataccacatccatgttacttagccggaacgaggcattataccaagcg

SEQ ID NO: 134 - DNA, 52 bp, Artificial
  Synthetic construct
ccggatattcattctttaatcattgtgaattaccttatgaatcgtcataaat

SEQ ID NO: 135 - DNA, 52 bp, Artificial
  Synthetic construct
agaccaggcgcatcagtcaggacgttgggaagaaaaatcccagagggggtaa

SEQ ID NO: 136 - DNA, 52 bp, Artificial
  Synthetic construct
cataagggaaccgcaacattattacaggtagaaagattccagacgacgataa

SEQ ID NO: 137 - DNA, 52 bp, Artificial
  Synthetic construct
tacagaggctttgtcgctgaggcttgcagggagttaaagcccaataggaacc

SEQ ID NO: 138 - DNA, 52 bp, Artificial
  Synthetic construct
gggtaaaatacgtgttgcgccgacaatgacaacaaccatacaacgcctgtag

SEQ ID NO: 139 - DNA, 52 bp, Artificial
  Synthetic construct
aaaagaatacactggtttatcagcttgctttcgaggtgaaagttttgtcgtc

SEQ ID NO: 140 - DNA, 52 bp, Artificial
  Synthetic construct
cgaaacaaagtaccacgttgaaaatctccaaaaaaaaggtgctaaacaactt

SEQ ID NO: 141 - DNA, 52 bp, Artificial
  Synthetic construct
ccgatatattcggaggactaaagactttttcatgaggaaatcttgacaagaa

SEQ ID NO: 142 - DNA, 52 bp, Artificial
  Synthetic construct
cttgataccgataaatgccactacgaaggcaccaacctaatgaacggtgtac

SEQ ID NO: 143 - DNA, 52 bp, Artificial
  Synthetic construct
ctttaattgtatcaaaacactcatctttgacccccagcggcagacggtcaat

SEQ ID NO: 144 - DNA, 52 bp, Artificial
  Synthetic construct
catgtaccgtaacagtaccgccaccctcagaaccgccacagcattgacagga

SEQ ID NO: 145 - DNA, 52 bp, Artificial
  Synthetic construct
cattccacagacaggttgatataagtatagcccggaataacaaataaatcct

SEQ ID NO: 146 - DNA, 52 bp, Artificial
  Synthetic construct
tttccagacgttattaggattagcggggttttgctcagtcgttccagtaagc

SEQ ID NO: 147 - DNA, 52 bp, Artificial
  Synthetic construct
tcaacagtttcagattattctgaaacatgaaagtattaaataagttttaacg

SEQ ID NO: 148 - DNA, 52 bp, Artificial
  Synthetic construct
actcaggaggtttactgagtttcgtcaccagtacaaactcgcccacgcataa

SEQ ID NO: 149 - DNA, 52 bp, Artificial
  Synthetic construct
gtgccgtcgagaggccctcatagttagcgtaacgatctaatttcttaaacag

SEQ ID NO: 150 - DNA, 52 bp, Artificial
  Synthetic construct
ctcaagagaaggagtaaatgaattttctgtatgggatttctccaaaaggagc

SEQ ID NO: 151 - DNA, 52 bp, Artificial
  Synthetic construct
ggttgaggcaggtgccgccaccctcagaaccgccacccttttgtcacaatca

SEQ ID NO: 152 - DNA, 52 bp, Artificial
  Synthetic construct
cattaaagccagacataatcaaaatcaccggaaccagaggggcgacattcaa

SEQ ID NO: 153 - DNA, 52 bp, Artificial
  Synthetic construct
gtcatacatggctttttcatcggcattttcggtcatagctcattaaaggtga

SEQ ID NO: 154 - DNA, 52 bp, Artificial
  Synthetic construct
gggtcagtgccttgtaatcagtagcgacagaatcaagttgccagcaaaatca

SEQ ID NO: 155 - DNA, 52 bp, Artificial
  Synthetic construct
cgcctccctcagacagacgattggccttgatattcacaaggtgtatcaccgt

SEQ ID NO: 156 - DNA, 52 bp, Artificial
  Synthetic construct
tttgccatcttttatggaaagcgcagtctctgaatttacaccaggcggataa

SEQ ID NO: 157 - DNA, 52 bp, Artificial
  Synthetic construct
agactgtagcgcgtttgatgatacaggagtgtactggtagaggctgagactc

SEQ ID NO: 158 - DNA, 52 bp, Artificial
  Synthetic construct
atagaaaattcatgttagcaaacgtagaaaatacatacagccagttacaaaa

SEQ ID NO: 159 - DNA, 52 bp, Artificial
  Synthetic construct
ccgattgagggagaacggaatacccaaaagaactggcatgattttttgttta

SEQ ID NO: 160 - DNA, 52 bp, Artificial
  Synthetic construct
attatcaccgtcaaagcagatagccgaacaaagttaccaacataaaaacagg

SEQ ID NO: 161 - DNA, 52 bp, Artificial
  Synthetic construct
ccagtagcaccatgaaacaatgaaatagcaatagctatccaaagtcagaggg

SEQ ID NO: 162 - DNA, 52 bp, Artificial
  Synthetic construct
tattacgcagtatatggtttaccagcgccaaagacaaaaccaccaccggaac

SEQ ID NO: 163 - DNA, 52 bp, Artificial
  Synthetic construct
gaaacgcaataatggaaggtaaatattgacggaaattatccccttattagcg

SEQ ID NO: 164 - DNA, 52 bp, Artificial
  Synthetic construct
ttttaagaaaagtccgacttgagccatttgggaattagatgcctttagcgtc

SEQ ID NO: 165 - DNA, 52 bp, Artificial
  Synthetic construct
taaacagccatatagttgctattttgcacccagctacaaaataagagaatat

SEQ ID NO: 166 - DNA, 52 bp, Artificial
  Synthetic construct
acgtcaaaaatgattttagcgaacctcccgacttgcgggcgacaataaacaa

SEQ ID NO: 167 - DNA, 52 bp, Artificial
  Synthetic construct
gaagcgcattagagcaagcaaatcagatatagaaggctttagataagtcctg

SEQ ID NO: 168 - DNA, 52 bp, Artificial
  Synthetic construct
taattgagcgctaaacaagcaagccgtttttattttcattagaaaccaatca

SEQ ID NO: 169 - DNA, 52 bp, Artificial
  Synthetic construct
ttaaatcaagatttatttatcccaatccaaataagaaacgattaagactcct

SEQ ID NO: 170 - DNA, 52 bp, Artificial
  Synthetic construct
agaacgcgaggcgaaatagcagcctttacagagagaatagaaggaaaccgag

SEQ ID NO: 171 - DNA, 52 bp, Artificial
  Synthetic construct
accgcgcccaatacgggagaattaactgaacaccctgaattaccgaagccct

SEQ ID NO: 172 - DNA, 52 bp, Artificial
  Synthetic construct
aaagtaccgacaaaacagtagggcttaattgagaatcgcctgtaaatcgtcg

SEQ ID NO: 173 - DNA, 52 bp, Artificial
  Synthetic construct
catgttcagctaagtttagtatcatatgcgttatacaaacgatagcttagat

SEQ ID NO: 174 - DNA, 52 bp, Artificial
  Synthetic construct
aacaagaaaaatataaggcgttaaataagaataaacacccataggtctgaga

SEQ ID NO: 175 - DNA, 52 bp, Artificial
  Synthetic construct
ataatcggctgtctttcatcttctgacctaaatttaatgactatatgtaaat

SEQ ID NO: 176 - DNA, 52 bp, Artificial
  Synthetic construct
aaagccaacgctcaaggtaaagtaattctgtccagacgaaggttttgaagcc

SEQ ID NO: 177 - DNA, 52 bp, Artificial
  Synthetic construct
ctagaaaaagccttgcagaacgcgcctgtttatcaacaaatccggtattcta

SEQ ID NO: 178 - DNA, 52 bp, Artificial
  Synthetic construct
accgtgtgataaaatatcccatcctaatttacgagcatgcgtaggaatcatt

SEQ ID NO: 179 - DNA, 52 bp, Artificial
  Synthetic construct
ctattaattaatttttaacaatttcatttgaattacctttggccctgagaga

SEQ ID NO: 180 - DNA, 52 bp, Artificial
  Synthetic construct
taagacgctgagactgagcaaaagaagatgatgaaacaagaaaatcctgttt

SEQ ID NO: 181 - DNA, 52 bp, Artificial
  Synthetic construct
gactacctttttattgaataccaagttacaaaatcgcgcaaagaatagcccg

SEQ ID NO: 182 - DNA, 52 bp, Artificial
  Synthetic construct
gctgatgcaaatcgtaacagtaccttttacatcgggagacactattaaagaa

SEQ ID NO: 183 - DNA, 52 bp, Artificial
  Synthetic construct
aaaattaattacattcccttagaatccttgaaaacatagttcttaccagtat

SEQ ID NO: 184 - DNA, 52 bp, Artificial
  Synthetic construct
tcatttcaattacagagtcaatagtgaatttatcaaaatggaatcataatta

SEQ ID NO: 185 - DNA, 52 bp, Artificial
  Synthetic construct
tcgcctgattgctacctccggcttaggttgggttatatagtttgaaataccg

SEQ ID NO: 186 - DNA, 52 bp, Artificial
  Synthetic construct
ttaaaattcgcattaaatttttgttaaatcagctcattttgtaaaacgacgg

SEQ ID NO: 187 - DNA, 52 bp, Artificial
  Synthetic construct
aggcaaggcaaagaattagcaaaattaagcaataaagcccgttaatattttg

SEQ ID NO: 188 - DNA, 52 bp, Artificial
  Synthetic construct
cataaatcaaaaatcaggtctttaccctgactattatagcaataaatcatac

SEQ ID NO: 189 - DNA, 52 bp, Artificial
  Synthetic construct
aggcttgccctgacgagaaacaccagaacgagtagtaaaaaacgagaatgac

SEQ ID NO: 190 - DNA, 52 bp, Artificial
  Synthetic construct
gatcgtcaccctcagcagcgaaagacagcatcggaacgacattcagtgaata

SEQ ID NO: 191 - DNA, 52 bp, Artificial
  Synthetic construct
accctcagagccaccaccctcattttcagggatagcaaggccgcttttgcgg

SEQ ID NO: 192 - DNA, 52 bp, Artificial
  Synthetic construct
ctcagagccgccaccagaaccaccaccagagccgccgcccctcagaaccgcc

SEQ ID NO: 193 - DNA, 52 bp, Artificial
  Synthetic construct
atataaaagaaacgcaaagacaccacggaataagtttatcagagccaccacc

SEQ ID NO: 194 - DNA, 52 bp, Artificial
  Synthetic construct
cttaccaacgctaacgagcgtctttccagagcctaattttaaaggtggcaac

SEQ ID NO: 195 - DNA, 52 bp, Artificial
  Synthetic construct
gccaacatgtaatttaggcagaggcattttcgagccagtttttatcctgaat

SEQ ID NO: 196 - DNA, 52 bp, Artificial
  Synthetic construct
agtacataaatcaatatatgtgagtgaataaccttgcttcatatttaacaac

SEQ ID NO: 197 - DNA, 52 bp, Artificial
  Synthetic construct
attaatgccggagcagtttgaggggacgacgacagtatcggcctcaggaaga

SEQ ID NO: 198 - DNA, 52 bp, Artificial
  Synthetic construct
atatgcaactaaaatcaccatcaatatgatattcaaccgttctagctgataa

SEQ ID NO: 199 - DNA, 52 bp, Artificial
  Synthetic construct
aggcatagtaagataattgctgaatataatgctgtagctcaacatgttttaa

SEQ ID NO: 200 - DNA, 52 bp, Artificial
  Synthetic construct
atccgcgacctgcttcaactaatgcagatacataacgccaaaaggaattacg

SEQ ID NO: 201 - DNA, 52 bp, Artificial
  Synthetic construct
ataataattttttaacggagatttgtatcatcgcctgataaattgtgtcgaa

SEQ ID NO: 202 - DNA, 52 bp, Artificial
  Synthetic construct
tatttcggaacctcggagtgagaatagaaaggaacaactaaaggaattgcga

SEQ ID NO: 203 - DNA, 52 bp, Artificial
  Synthetic construct
cgatagcagcaccgagtaacagtgcccgtataaacagttaatgccccctgcc

SEQ ID NO: 204 - DNA, 52 bp, Artificial
  Synthetic construct
ataataagagcaataccattagcaaggccggaaacgtcaccaatgaaaccat

SEQ ID NO: 205 - DNA, 52 bp, Artificial
  Synthetic construct
cgcactcatcgagatatcagagagataacccacaagaattgagttaagccca

SEQ ID NO: 206 - DNA, 52 bp, Artificial
  Synthetic construct
atattttagttaatttccttatcattccaagaacgggtattaaaccaagtac

SEQ ID NO: 207 - DNA, 52 bp, Artificial
  Synthetic construct
agatgaatatacacaatcgcaagacaaagaacgcgagaaaactttttcaaat

SEQ ID NO: 208 - DNA, 52 bp, Artificial
  Synthetic construct
gttgcagcaagcggtccacgctggtttgccccagcaggcacatcaagaaaac

SEQ ID NO: 209 - DNA, 52 bp, Artificial
  Synthetic construct
gatggtggttccgaaatcggcaaaatcccttataaatcaagaggcgaattat

SEQ ID NO: 210 - DNA, 52 bp, Artificial
  Synthetic construct
agatagggttgagtgttgttccagtttggaacaagagtcaacaataacggat

SEQ ID NO: 211 - DNA, 52 bp, Artificial
  Synthetic construct
ccagtgccaagctttctcaggagaagccagggtggatgttcttctaagtggt

SEQ ID NO: 212 - DNA, 52 bp, Artificial
  Synthetic construct
ttaagttgggtaacgccagggttttcccagtcacgacgttttaaccaatagg

SEQ ID NO: 213 - DNA, 52 bp, Artificial
  Synthetic construct
ggcctcttcgctattacgccagctggcgaaagggggatgccagctttcatca

SEQ ID NO: 214 - DNA, 52 bp, Artificial
  Synthetic construct
agcgccattcgccattcaggctgcgcaactgttgggaaggtgggaacaaacg

SEQ ID NO: 215 - DNA, 52 bp, Artificial
  Synthetic construct
tcgcactccagccagctttccggcaccgcttctggtgcctgggcgcatcgta

SEQ ID NO: 216 - DNA, 62 bp, Artificial
  Synthetic construct
ttaaaattcgcattaaatttttgttaaatcagctcattttgtaaaacgacggaacttgacct

SEQ ID NO: 217 - DNA, 62 bp, Artificial
  Synthetic construct
aggcaaggcaaagaattagcaaaattaagcaataaagcccgttaatattttgtgctcgtgtt

SEQ ID NO: 218 - DNA, 62 bp, Artificial
  Synthetic construct
cataaatcaaaaatcaggtctttaccctgactattatagcaataaatcatacgcacgccaaa

SEQ ID NO: 219 - DNA, 62 bp, Artificial
  Synthetic construct
aggcttgccctgacgagaaacaccagaacgagtagtaaaaaacgagaatgacgagatgcttc

SEQ ID NO: 220 - DNA, 62 bp, Artificial
  Synthetic construct
gatcgtcaccctcagcagcgaaagacagcatcggaacgacattcagtgaatattgtggaact

SEQ ID NO: 221 - DNA, 62 bp, Artificial
  Synthetic construct
accctcagagccaccaccctcattttcagggatagcaaggccgcttttgcggcgacaacgca

SEQ ID NO: 222 - DNA, 62 bp, Artificial
  Synthetic construct
ctcagagccgccaccagaaccaccaccagagccgccgcccctcagaaccgccggatctacgt

SEQ ID NO: 223 - DNA, 62 bp, Artificial
  Synthetic construct
atataaaagaaacgcaaagacaccacggaataagtttatcagagccaccacccggagttgct

SEQ ID NO: 224 - DNA, 62 bp, Artificial
  Synthetic construct
cttaccaacgctaacgagcgtctttccagagcctaattttaaaggtggcaacgcgacattgg

SEQ ID NO: 225 - DNA, 62 bp, Artificial
  Synthetic construct
gccaacatgtaatttaggcagaggcattttcgagccagtttttatcctgaatgatctgtcag

SEQ ID NO: 226 - DNA, 62 bp, Artificial
  Synthetic construct
agtacataaatcaatatatgtgagtgaataaccttgcttcatatttaacaaccgaaaaacat

SEQ ID NO: 227 - DNA, 62 bp, Artificial
  Synthetic construct
attaatgccggagcagtttgaggggacgacgacagtatcggcctcaggaagaccgtccccga

SEQ ID NO: 228 - DNA, 62 bp, Artificial
  Synthetic construct
atatgcaactaaaatcaccatcaatatgatattcaaccgttctagctgataaggcggacact

SEQ ID NO: 229 - DNA, 62 bp, Artificial
  Synthetic construct
aggcatagtaagataattgctgaatataatgctgtagctcaacatgttttaagattgacgcg

SEQ ID NO: 230 - DNA, 62 bp, Artificial
  Synthetic construct
atccgcgacctgcttcaactaatgcagatacataacgccaaaaggaattacggttttgtaga

SEQ ID NO: 231 - DNA, 62 bp, Artificial
  Synthetic construct
ataataattttttaacggagatttgtatcatcgcctgataaattgtgtcgaaaggttagggg

SEQ ID NO: 232 - DNA, 62 bp, Artificial
  Synthetic construct
tatttcggaacctcggagtgagaatagaaaggaacaactaaaggaattgcgaaataggttag

SEQ ID NO: 233 - DNA, 62 bp, Artificial
  Synthetic construct
cgatagcagcaccgagtaacagtgcccgtataaacagttaatgccccctgccattgagtggc

SEQ ID NO: 234 - DNA, 62 bp, Artificial
  Synthetic construct
ataataagagcaataccattagcaaggccggaaacgtcaccaatgaaaccatttaagaatgt

SEQ ID NO: 235 - DNA, 62 bp, Artificial
  Synthetic construct
cgcactcatcgagatatcagagagataacccacaagaattgagttaagcccaattatagtgt

SEQ ID NO: 236 - DNA, 62 bp, Artificial
  Synthetic construct
atattttagttaatttccttatcattccaagaacgggtattaaaccaagtacagtaatctct

SEQ ID NO: 237 - DNA, 62 bp, Artificial
  Synthetic construct
agatgaatatacacaatcgcaagacaaagaacgcgagaaaactttttcaaatgattaacggt

SEQ ID NO: 238 - DNA, 62 bp, Artificial
  Synthetic construct
aggtcaagttccgtcgttttacaaaatgagctgatttaacaaaaatttaatgcgaattttaa

SEQ ID NO: 239 - DNA, 62 bp, Artificial
  Synthetic construct
aacacgagcacaaaatattaacgggctttattgcttaattttgctaattctttgccttgcct

SEQ ID NO: 240 - DNA, 62 bp, Artificial
  Synthetic construct
tttggcgtgcgtatgatttattgctataatagtcagggtaaagacctgatttttgatttatg

SEQ ID NO: 241 - DNA, 62 bp, Artificial
  Synthetic construct
gaagcatctcgtcattctcgttttttactactcgttctggtgtttctcgtcagggcaagcct

SEQ ID NO: 242 - DNA, 62 bp, Artificial
  Synthetic construct
agttccacaatattcactgaatgtcgttccgatgctgtctttcgctgctgagggtgacgatc

SEQ ID NO: 243 - DNA, 62 bp, Artificial
  Synthetic construct
tgcgttgtcgccgcaaaagcggccttgctatccctgaaaatgagggtggtggctctgagggt

SEQ ID NO: 244 - DNA, 62 bp, Artificial
  Synthetic construct
acgtagatccggcggttctgaggggcggcggctctggtggtggttctggtggcggctctgag

SEQ ID NO: 245 - DNA, 62 bp, Artificial
  Synthetic construct
agcaactccgggtggtggctctgataaacttattccgtggtgtctttgcgtttcttttatat

SEQ ID NO: 246 - DNA, 62 bp, Artificial
  Synthetic construct
ccaatgtcgcgttgccacctttaaaattaggctctggaaagacgctcgttagcgttggtaag

SEQ ID NO: 247 - DNA, 62 bp, Artificial
  Synthetic construct
ctgacagatcattcaggataaaaactggctcgaaaatgcctctgcctaaattacatgttggc

SEQ ID NO: 248 - DNA, 62 bp, Artificial
  Synthetic construct
atgtttttcggttgttaaatatgaagcaaggttattcactcacatatattgatttatgtact

SEQ ID NO: 249 - DNA, 62 bp, Artificial
  Synthetic construct
tcggggacggtcttcctgaggccgatactgtcgtcgtcccctcaaactgctccggcattaat

SEQ ID NO: 250 - DNA, 62 bp, Artificial
  Synthetic construct
agtgtccgccttatcagctagaacggttgaatatcatattgatggtgattttagttgcatat

SEQ ID NO: 251 - DNA, 62 bp, Artificial
  Synthetic construct
cgcgtcaatcttaaaacatgttgagctacagcattatattcagcaattatcttactatgcct

SEQ ID NO: 252 - DNA, 62 bp, Artificial
  Synthetic construct
tctacaaaaccgtaattccttttggcgttatgtatctgcattagttgaagcaggtcgcggat

SEQ ID NO: 253 - DNA, 62 bp, Artificial
  Synthetic construct
cccctaacctttcgacacaatttatcaggcgatgatacaaatctccgttaaaaaattattat

SEQ ID NO: 254 - DNA, 62 bp, Artificial
  Synthetic construct
ctaacctatttcgcaattcctttagttgttcctttctattctcactccgaggttccgaaata

SEQ ID NO: 255 - DNA, 62 bp, Artificial
  Synthetic construct
gccactcaatggcagggggcattaactgtttatacgggcactgttactcggtgctgctatcg

SEQ ID NO: 256 - DNA, 62 bp, Artificial
  Synthetic construct
acattcttaaatggtttcattggtgacgtttccggccttgctaatggtattgctcttattat

SEQ ID NO: 257 - DNA, 62 bp, Artificial
  Synthetic construct
acactataattgggcttaactcaattcttgtgggttatctctctgatatctcgatgagtgcg

SEQ ID NO: 258 - DNA, 62 bp, Artificial
  Synthetic construct
agagattactgtacttggtttaatacccgttcttggaatgataaggaaattaactaaaatat

SEQ ID NO: 259 - DNA, 62 bp, Artificial
  Synthetic construct
accgttaatcatttgaaaaagttttctcgcgttctttgtcttgcgattgtgtatattcatct

SEQ ID NO: 260 - DNA, 62 bp, Artificial
  Synthetic construct
gttgcagcaagcggtccacgctggtttgccccagcaggcacatcaagaaaacctccccgggt

SEQ ID NO: 261 - DNA, 62 bp, Artificial
  Synthetic construct
gatggtggttccgaaatcggcaaaatcccttataaatcaagaggcgaattatgtggctcctt

SEQ ID NO: 262 - DNA, 62 bp, Artificial
  Synthetic construct
agatagggttgagtgttgttccagtttggaacaagagtcaacaataacggatcatctgacaa

SEQ ID NO: 263 - DNA, 62 bp, Artificial
  Synthetic construct
cgtggactccaacgtcaaagggcgaaaaaccgtctatcacaggtttaacgtcctatcgccat

SEQ ID NO: 264 - DNA, 62 bp, Artificial
  Synthetic construct
ttaagttgggtaacgccagggttttcccagtcacgacgttttaaccaataggcgattgtttc

SEQ ID NO: 265 - DNA, 62 bp, Artificial
  Synthetic construct
ggcctcttcgctattacgccagctggcgaaagggggatgccagctttcatcatgcggacggt

SEQ ID NO: 266 - DNA, 62 bp, Artificial
  Synthetic construct
agcgccattcgccattcaggctgcgcaactgttgggaaggtgggaacaaacggttgtcctca

SEQ ID NO: 267 - DNA, 62 bp, Artificial
  Synthetic construct
tcgcactccagccagctttccggcaccgcttctggtgcctgggcgcatcgtatagtttgggc

SEQ ID NO: 268 - DNA, 62 bp, Artificial
  Synthetic construct
acccggggaggttttcttgatgtgcctgctggggcaaaccagcgtggaccgcttgctgcaac

SEQ ID NO: 269 - DNA, 62 bp, Artificial
  Synthetic construct
aaggagccacataattcgcctcttgatttataagggattttgccgatttcggaaccaccatc

SEQ ID NO: 270 - DNA, 62 bp, Artificial
  Synthetic construct
ttgtcagatgatccgttattgttgactcttgttccaaactggaacaacactcaaccctatct

SEQ ID NO: 271 - DNA, 62 bp, Artificial
  Synthetic construct
atggcgataggacgttaaacctgtgatagacggtttttcgccctttgacgttggagtccacg

SEQ ID NO: 272 - DNA, 62 bp, Artificial
  Synthetic construct
gaaacaatcgcctattggttaaaacgtcgtgactgggaaaaccctggcgttacccaacttaa

SEQ ID NO: 273 - DNA, 62 bp, Artificial
  Synthetic construct
accgtccgcatgatgaaagctggcatccccctttcgccagctggcgtaatagcgaagaggcc

SEQ ID NO: 274 - DNA, 62 bp, Artificial
  Synthetic construct
tgaggacaaccgtttgttcccaccttcccaacagttgcgcagcctgaatggcgaatggcgct

SEQ ID NO: 275 - DNA, 62 bp, Artificial
  Synthetic construct
gcccaaactatacgatgcgcccaggcaccagaagcggtgccggaaagctggctggagtgcga

SEQ ID NO: 276 - DNA, 52 bp, Artificial
  Synthetic construct
ttaagttgggtaagtccacgctggtttgccccagcaggcacatcaagaaaac

SEQ ID NO: 277 - DNA, 52 bp, Artificial
  Synthetic construct
ggcctcttcgctaaaatcggcaaaatcccttataaatcaagaggcgaattat

SEQ ID NO: 278 - DNA, 52 bp, Artificial
  Synthetic construct
agcgccattcgcctgttgttccagtttggaacaagagtcaacaataacggat

SEQ ID NO: 279 - DNA, 52 bp, Artificial
  Synthetic construct
gttgcagcaagcgcgccagggttttcccagtcacgacgttttaaccaatagg

SEQ ID NO: 280 - DNA, 52 bp, Artificial
  Synthetic construct
gatggtggttccgttacgccagctggcgaaagggggatgccagctttcatca

SEQ ID NO: 281 - DNA, 52 bp, Artificial
  Synthetic construct
agatagggttgagattcaggctgcgcaactgttgggaaggtgggaacaaacg

SEQ ID NO: 282 - DNA, 52 bp, Artificial
  Synthetic construct
cgtggactccaacagctttccggcaccgcttctggtgcctgggcgcatcgta

SEQ ID NO: 283 - DNA, 56 bp, Artificial
  Synthetic construct
tttacccacgacagtttacgatggcatggatgtcctcgatcctgtcaaacttattt

SEQ ID NO: 284 - DNA, 56 bp, Artificial
  Synthetic construct
ttttccttccgaagcgtgcaaatccgtccgagtctcacatcctcacggccctattt

SEQ ID NO: 285 - DNA, 64 bp, Artificial
  Synthetic construct
tttaacctactctatccaccacgggttggaccgggtacgaacataccgtagtcttcagggtttt

SEQ ID NO: 286 - DNA, 59 bp, Artificial
  Synthetic construct
tttcggaaggaggtgatctcgtcgcatccatggatcggagttgacaggcttaacacttt

SEQ ID NO: 287 - DNA, 59 bp, Artificial
  Synthetic construct
tttagtaggttgctaatccatactctcggacgcttaagagccgtgaggttcccgtattt

SEQ ID NO: 288 - DNA, 51 bp, Artificial
  Synthetic construct
tttatacatccaaactggtccaacgtgatagacggtatgttaggctccttt

SEQ ID NO: 289 - DNA, 48 bp, Artificial
  Synthetic construct
tttccttgggagggcttcatcatgccatcgtaaactgtcgtgggtttt

SEQ ID NO: 290 - DNA, 64 bp, Artificial
  Synthetic construct
ttttcccaaggagctaatactccaaatcgattgatcatggcaaagggcatttaggccctgtttt

SEQ ID NO: 291 - DNA, 64 bp, Artificial
  Synthetic construct
tttggacacgaacgctgacgaaaaaaatggaggcttcaataaccgcccatcactaatccgattt

SEQ ID NO: 292 - DNA, 72 bp, Artificial
  Synthetic construct
tttcatacatgcacgcacactattgtcttttcgatgtagcaaatcccacgactggatggctcaattcgcttt

SEQ ID NO: 293 - DNA, 59 bp, Artificial
  Synthetic construct
tttgtcagcgtgagattccgtactccatgatcctggttccgcctaaatgtaattcgttt

SEQ ID NO: 294 - DNA, 59 bp, Artificial
  Synthetic construct
ttttgtgcgtggcttacgcgctgaattgaagctcgtagtcttagtgatggcgggtattt

SEQ ID NO: 295 - DNA, 51 bp, Artificial
  Synthetic construct
tttcttgcgagggtccgctacatcgcacttcgtccagtcgcacctgtgttt

SEQ ID NO: 296 - DNA, 48 bp, Artificial
  Synthetic construct
tttctagtccacatcgcggcgcataatcgatttggagtattagctttt

SEQ ID NO: 297 - DNA, 64 bp, Artificial
  Synthetic construct
ttttggactaggtgaaccgagttgactttctggccaactgccacacacaggggacctgtgtttt

SEQ ID NO: 298 - DNA, 64 bp, Artificial
  Synthetic construct
tttcgcgaatgcttaatccttctggtcgtaatttgtccaaatttgcactgcaggccgccgcttt

SEQ ID NO: 299 - DNA, 72 bp, Artificial
  Synthetic construct
tttactaggtaatttagatttgcgtcccggttgcgtctctgcaagtgcgcggctttggtaatactctacttt

SEQ ID NO: 300 - DNA, 59 bp, Artificial
  Synthetic construct
tttggattaaggtggctgaatgtgcagttggcaaattcacggtcccctcatgaattttt

SEQ ID NO: 301 - DNA, 59 bp, Artificial
  Synthetic construct
tttatctaaattgataagcgagtattggacaaatgagcagggcctgcatctcggatttt

SEQ ID NO: 302 - DNA, 51 bp, Artificial
  Synthetic construct
tttggggattgacgcaagagacgctatatatgaaagccgcgacaattattt

SEQ ID NO: 303 - DNA, 48 bp, Artificial
  Synthetic construct
tttacacctcacgaatggctcaagcagaaagtcaactcggttcacttt

SEQ ID NO: 304 - DNA, 64 bp, Artificial
  Synthetic construct
ttttgaggtgtacgagggcacgaagcggtaaccgaagcccctgcactccagaaagtggttattt

SEQ ID NO: 305 - DNA, 64 bp, Artificial
  Synthetic construct
ttttctcgaggacaggtgaaagaagatgtgaattgacattgggttacattgacagagagatttt

SEQ ID NO: 306 - DNA, 72 bp, Artificial
  Synthetic construct
tttccgtcgcgacaccgccggagttcgtctactgattaacggtacgtagcgttgtagcaccgttgagcattt

SEQ ID NO: 307 - DNA, 72 bp, Artificial
  Synthetic construct
ttttcacctgtgaacaaagtgcaagggcttcggattcgtaactttctgtggaagagtatcacagtgcgattt

SEQ ID NO: 308 - DNA, 72 bp, Artificial
  Synthetic construct
tttggcggtgtgggtgacgattataatgtcaactttaagttctgtcaactgcaattgcgtcagcggttgttt

SEQ ID NO: 309 - DNA, 64 bp, Artificial
  Synthetic construct
tttgaccgtagtcgaagttaatcaattaaaaatacaacgcccctatttacggcttaactgattt

SEQ ID NO: 310 - DNA, 48 bp, Artificial
  Synthetic construct
tttaagacataaagcaagtattgtgttaccgcttcgtgccctcgtttt

SEQ ID NO: 311 - DNA, 48 bp, Artificial
  Synthetic construct
ttttatcgcgatactatacgaatcacaatacttgctttatgtcttttt

SEQ ID NO: 312 - DNA, 40 bp, Artificial
  Synthetic construct
ttttgtcagcacttgaacttaaagtacgtgcgacgtcttt

SEQ ID NO: 313 - DNA, 40 bp, Artificial
  Synthetic construct
ttttctgagaacggtttttttaataaggcccaacccgttt

SEQ ID NO: 314 - DNA, 22 bp, Artificial
  Synthetic construct
tttcacactcatgtacccattt

SEQ ID NO: 315 - DNA, 30 bp, Artificial
  Synthetic construct
tttgcctgcaagagttcctgtgtggtgttt

SEQ ID NO: 316 - DNA, 30 bp, Artificial
  Synthetic construct
tttagtggaaacagtaagtgaatttatttt

SEQ ID NO: 317 - DNA, 45 bp, Artificial
  Synthetic construct
ttttccacttgcaggccaacactttgagacgacgagatcaccttt

SEQ ID NO: 318 - DNA, 45 bp, Artificial
  Synthetic construct
ttttcacttttccactcctcctaattctaagtatggattagcttt

SEQ ID NO: 319 - DNA, 32 bp, Artificial
  Synthetic construct
tttcccatagaccgagagtttggatgtatttt

SEQ ID NO: 320 - DNA, 35 bp, Artificial
  Synthetic construct
tttatggtgcgcttgagctggaaaaatgggtattt

SEQ ID NO: 321 - DNA, 43 bp, Artificial
  Synthetic construct
tttgctagtcatatggagtaggcgaccaaaccgctatttattt

SEQ ID NO: 322 - DNA, 43 bp, Artificial
  Synthetic construct
tttctaggatgagattactgtcatttccgactcggggcaattt

SEQ ID NO: 323 - DNA, 35 bp, Artificial
  Synthetic construct
tttcgtgacgcaccatgcacgaacatttttccttt

SEQ ID NO: 324 - DNA, 43 bp, Artificial
  Synthetic construct
tttacggtactccatagactacgaagcggttttggcatgcttt

SEQ ID NO: 325 - DNA, 43 bp, Artificial
  Synthetic construct
tttgaacgagtaatctgagtgttcccgagtcgaatggaggttt

SEQ ID NO: 326 - DNA, 74 bp, Artificial
  Synthetic construct
ttttagtctgttgactagctaagttccccacgccgcctcgtgtccaacggtggcacctgatttgcacgcttttt

SEQ ID NO: 327 - DNA, 74 bp, Artificial
  Synthetic construct
ttttagcttagcatcctagtagggataacattttcagcatgtatggaaagcaaaggttccgtggtggatagttt

SEQ ID NO: 328 - DNA, 40 bp, Artificial
  Synthetic construct
tttagtctaacaccctgaagactataagctgtggggattt

SEQ ID NO: 329 - DNA, 79 bp, Artificial
  Synthetic construct
ttttcagttcgtagtcacagactacatttagtacggaatctcggcggcgtggggactccgatccatgatgaagcccttt

SEQ ID NO: 330 - DNA, 79 bp, Artificial
  Synthetic construct
ttttgtgcgaacactcctaagctacccactcagcgcgtaagcctgaaaatgttatctcttaagaggtgccaccgttttt

SEQ ID NO: 331 - DNA, 66 bp, Artificial
  Synthetic construct
tttgttagactcacacggaccctcgcaagtccccacagcttatctatcacaacctttgctttcttt

SEQ ID NO: 332 - DNA, 35 bp, Artificial
  Synthetic construct
tttctaccgagggaggtgcacctcgaatctcattt

SEQ ID NO: 333 - DNA, 43 bp, Artificial
  Synthetic construct
tttggcgggtagcctcgtgataagccttatcttgcactgcttt

SEQ ID NO: 334 - DNA, 43 bp, Artificial
  Synthetic construct
tttcgatttagagcttgaaggtataaggaataaatgttgtttt

SEQ ID NO: 335 - DNA, 35 bp, Artificial
  Synthetic construct
ttttacgcctcggtagtgtccctgttcgaggtttt

SEQ ID NO: 336 - DNA, 43 bp, Artificial
  Synthetic construct
tttctaaccacgaggcgcctacccgcaagatacatactttttt

SEQ ID NO: 337 - DNA, 43 bp, Artificial
  Synthetic construct
tttgcacattcaagctgcctggacatttattcgtgtggcattt

SEQ ID NO: 338 - DNA, 74 bp, Artificial
  Synthetic construct
ttttagacacgtacccgccacagggcggtgaccgcggcattcgcgacctatgccaaaactccatttttttcttt

SEQ ID NO: 339 - DNA, 74 bp, Artificial
  Synthetic construct
tttgtacgacactaaatcgtcggatatgcccggagtgtacctagtgtaatagcggcttgaaaagacaatagttt

SEQ ID NO: 340 - DNA, 40 bp, Artificial
  Synthetic construct
tttcggtctttgcgaattgagccatgctaaaccgcaattt

SEQ ID NO: 341 - DNA, 79 bp, Artificial
  Synthetic construct
tttaccacgggtaggccgtgtctaacttacacattcagccacccgcggtcaccgcggaaccagatgcgccgcgatgttt

SEQ ID NO: 342 - DNA, 79 bp, Artificial
  Synthetic construct
tttgacaagtccaggctgtcgtacgtacctactcgcttatcacactccgggcatagactacgattttggcataggtttt

SEQ ID NO: 343 - DNA, 66 bp, Artificial
  Synthetic construct
tttaaagaccgttgtttgcgtcaatccccttgcggtttagcacgaagtgcaagccgctattacttt

SEQ ID NO: 344 - DNA, 35 bp, Artificial
  Synthetic construct
tttgtatactgggaagactttgtctttatcgtttt

SEQ ID NO: 345 - DNA, 43 bp, Artificial
  Synthetic construct
tttcgatacttctctacggctcggaaattataggtttattttt

SEQ ID NO: 346 - DNA, 43 bp, Artificial
  Synthetic construct
ttttattctttactatttattgtttcaatttcctccgccattt

SEQ ID NO: 347 - DNA, 35 bp, Artificial
  Synthetic construct
tttaggttcagtataccccggaggaaagacaattt

SEQ ID NO: 348 - DNA, 43 bp, Artificial
  Synthetic construct
ttttacacccgtagagggtctaaaacctataaaagaacctttt

SEQ ID NO: 349 - DNA, 43 bp, Artificial
  Synthetic construct
ttttaaggtaaatagtgacggattgaggaaatattgttctttt

SEQ ID NO: 350 - DNA, 74 bp, Artificial
  Synthetic construct
ttttctctgggaagtatcgacacagtgtagtctctaccctcgagatcgctaccggggaattacgaccagaattt

SEQ ID NO: 351 - DNA, 74 bp, Artificial
  Synthetic construct
tttcaattataaaagaatagcggcgtccatcagagcccgcgacggttactcatcggccaaccgggacgcaattt

SEQ ID NO: 352 - DNA, 40 bp, Artificial
  Synthetic construct
tttggtcccgtgtagagtattaccgtacttcattgtgttt

SEQ ID NO: 353 - DNA, 79 bp, Artificial
  Synthetic construct
tttctcgctttagacccccagagagttaattgcactttgttcgtagagactacacgtgaatttcttgagccattcgttt

SEQ ID NO: 354 - DNA, 79 bp, Artificial
  Synthetic construct
tttcatcgaatccgtctataattggacggataatcgtcacccggctctgatggacctgctcattccccggtagcgattt

SEQ ID NO: 355 - DNA, 66 bp, Artificial
  Synthetic construct
tttacgggaccgagagttcgactacggtccacaatgaagtaccatatataggccgatgagtaattt

SEQ ID NO: 356 - DNA, 22 bp, Artificial
  Synthetic construct
ttttcgcactgtcgcatgtttt

SEQ ID NO: 357 - DNA, 30 bp, Artificial
  Synthetic construct
tttcaagattccaaccgctcttggttcttt

SEQ ID NO: 358 - DNA, 30 bp, Artificial
  Synthetic construct
tttacagaggatcagttaatctgtgctttt

SEQ ID NO: 359 - DNA, 66 bp, Artificial
  Synthetic construct
tttgaatcttgtaacctagtatcgcgatagcgtaaacgacgtcgcacgtattcacatcttcttttt

SEQ ID NO: 360 - DNA, 66 bp, Artificial
  Synthetic construct
ttttcctctgtatctctcaagtgctgacatggggtcccgggttgggccttgtagacgaactccttt

SEQ ID NO: 361 - DNA, 32 bp, Artificial
  Synthetic construct
ttttgctcaacggtgcaaccgttctcagattt

SEQ ID NO: 362 - DNA, 72 bp, Artificial
  Synthetic construct
ttttgggtacaccatgcccacagccttaagtggtggtacaaacgcaaggatctttacgcgttatgaaccttt

SEQ ID NO: 363 - DNA, 72 bp, Artificial
  Synthetic construct
tttaggaactccgtgttcgtattctggcgttagatcgcaggcagggattcccttgtgacgacgagattcttt

SEQ ID NO: 364 - DNA, 64 bp, Artificial
  Synthetic construct
tttacttactgttcaccaccagttactaattgcacgctactcttgaggcgctgtgtttttcttt

SEQ ID NO: 365 - DNA, 64 bp, Artificial
  Synthetic construct
tttctcttcacttaagatagccagcttgcgttagcaacgtagactaattgaatgacaacttttt

SEQ ID NO: 366 - DNA, 64 bp, Artificial
  Synthetic construct
tttgttcctaacgccaatgtggcaatccctgctcggctgcatggtggttcatatgcgttccttt

SEQ ID NO: 367 - DNA, 72 bp, Artificial
  Synthetic construct
tttaccgttcccagggcaattagtccacaagacctcaagaccagtcgagacatgaatctcgacagtggattt

SEQ ID NO: 368 - DNA, 74 bp, Artificial
  Synthetic construct
tttagctcaaggcggataacgggtgctgtgggcatgggtataccgggacaatcgaggatctcaaagtgttgttt

SEQ ID NO: 369 - DNA, 61 bp, Artificial
  Synthetic construct
tttagcgtggagaatacgaacacggtggacgagctagatgtgagatagaattaggaggttt

SEQ ID NO: 370 - DNA, 61 bp, Artificial
  Synthetic construct
ttttccatgtgaactggtggtgaaagtgaggagggcatcgtacccctcggtctatgggttt

SEQ ID NO: 371 - DNA, 66 bp, Artificial
  Synthetic construct
tttaagacctggctatacccgttatccgctcacggtgttaagtgtcccggtatactgagtgtgttt

SEQ ID NO: 372 - DNA, 53 bp, Artificial
  Synthetic construct
tttagaactgccacattccacgctaggataccgttacgggaactagctcgttt

SEQ ID NO: 373 - DNA, 53 bp, Artificial
  Synthetic construct
tttcccagtcttgtggcacatggaacagccgttcggagcctatgccctccttt

SEQ ID NO: 374 - DNA, 59 bp, Artificial
  Synthetic construct
tttgtaatcgcctgataacatccagtatggcggcggaggatgcgcccctgcgatagttt

SEQ ID NO: 375 - DNA, 59 bp, Artificial
  Synthetic construct
tttgacatcgctggtttcagctcgcgcagagaggcctatcggtggcagatcgttgcttt

SEQ ID NO: 376 - DNA, 51 bp, Artificial
  Synthetic construct
tttaagttacgaccatcgggtcatacggggaggagacagaatctactcttt

SEQ ID NO: 377 - DNA, 64 bp, Artificial
  Synthetic construct
tttcattagttatcagcccgtgggcgccgccagagacgggtatgtgatggtttgtgctgagttt

SEQ ID NO: 378 - DNA, 64 bp, Artificial
  Synthetic construct
tttgtcgatgaaaccaagatgcatgcctctctatcccgacttcaactatcgcacaaagcatttt

SEQ ID NO: 379 - DNA, 72 bp, Artificial
  Synthetic construct
tttacccttaccacccccgatggtatagagagctcctccctttaatgcctaccgcaacgatagtaccgtttt

SEQ ID NO: 380 - DNA, 66 bp, Artificial
  Synthetic construct
tttgcacctccattccgacggggggcgattactacccctctgttctacaagccctttgaaatgttt

SEQ ID NO: 381 - DNA, 53 bp, Artificial
  Synthetic construct
tttcataatccgcgatgtctaaatactgaccagatgagggcggttgtgggttt

SEQ ID NO: 382 - DNA, 53 bp, Artificial
  Synthetic construct
tttggtatagtcgtaacttttgccgcacaagcacacatgggatttgtgtgttt

SEQ ID NO: 383 - DNA, 66 bp, Artificial
  Synthetic construct
tttgaattcccacgggcccccgtcggaatgcgtacgaattacttgtagaacagaggttcgtgcttt

SEQ ID NO: 384 - DNA, 53 bp, Artificial
  Synthetic construct
tttgagacatgcatctggattatgcggatgttagtacccgcctcatctggttt

SEQ ID NO: 385 - DNA, 53 bp, Artificial
  Synthetic construct
ttttgaatctctctatactataccgctcctgtgccacaggtgtgtgtgctttt

SEQ ID NO: 386 - DNA, 59 bp, Artificial
  Synthetic construct
tttacgtagggaattctacgtttacaactgggaagcactgcgagaaaagctagcccttt

SEQ ID NO: 387 - DNA, 59 bp, Artificial
  Synthetic construct
tttttggtaggatggcagttgacaacacagtgtcagggatgctggccaataagtcattt

SEQ ID NO: 388 - DNA, 51 bp, Artificial
  Synthetic construct
tttttcacgactgtttttacaagtactacttgcaattacgcacacaatttt

SEQ ID NO: 389 - DNA, 64 bp, Artificial
  Synthetic construct
tttagggagtagaattggtttcagcttcccagataaccgcaagaagttgccgggccccacgttt

SEQ ID NO: 390 - DNA, 64 bp, Artificial
  Synthetic construct
tttgcagcactgccattctggctatgacactgtagacaatgaatggggctagcaacagggtttt

SEQ ID NO: 391 - DNA, 72 bp, Artificial
  Synthetic construct
ttttttaaaagcagtttaaaaacaggtctcgtttgcaagtgccttcgtccagatgacttattttcaagtttt

SEQ ID NO: 392 - DNA, 66 bp, Artificial
  Synthetic construct
tttagtcttccaactgaagataacccctacgttgagagcgtctgtcaaaggtgtgtggtaagtttt

SEQ ID NO: 393 - DNA, 53 bp, Artificial
  Synthetic construct
tttttctcaggcctaccaagcagtgtggtactttccagtgcaaatggtacttt

SEQ ID NO: 394 - DNA, 53 bp, Artificial
  Synthetic construct
ttttgaggccggtcgtgaaacaacttgtcgataggtggcacttgcaacaattt

SEQ ID NO: 395 - DNA, 66 bp, Artificial
  Synthetic construct
tttggacactgaaaccgttatcttcagttaacctaattcatgctttgacagacgccagggacattt

SEQ ID NO: 396 - DNA, 53 bp, Artificial
  Synthetic construct
ttttgtcgtagccagacctgagaacgcgcgtgtaatccgagatggaaagtttt

SEQ ID NO: 397 - DNA, 53 bp, Artificial
  Synthetic construct
tttgcgagacgagacccggcctcacctacccttataattgtccacctatcttt

SEQ ID NO: 398 - DNA, 59 bp, Artificial
  Synthetic construct
tttaatatgcttgtaacccacgacgttctcttaagttgtagtatcgtttatagcgcttt

SEQ ID NO: 399 - DNA, 59 bp, Artificial
  Synthetic construct
tttgcactcaccccccggtgagcgggttgttttcgtgccgccttccttggacagaattt

SEQ ID NO: 400 - DNA, 51 bp, Artificial
  Synthetic construct
tttaaataatccttataagccttcctgcaatttgtgaatgacttttccttt

SEQ ID NO: 401 - DNA, 56 bp, Artificial
  Synthetic construct
tttgaccagggttacacgcacactcttaagagataagttcaaccacccgaacgttt

SEQ ID NO: 402 - DNA, 56 bp, Artificial
  Synthetic construct
tttccagtaccgggggataacagtcgaaaacagcacactctcgaagcgctatattt

SEQ ID NO: 403 - DNA, 64 bp, Artificial
  Synthetic construct
tttggcaattgttcgccttataaggagtccgcacaaattgaggtgcggggtgcttctgtccttt

SEQ ID NO: 404 - DNA, 66 bp, Artificial
  Synthetic construct
tttacatgcgaaggtcttgtcgtaagcatattacgatcatcacacttgtcgagtgcagttaacttt

SEQ ID NO: 405 - DNA, 53 bp, Artificial
  Synthetic construct
tttacacgagtgtgagtgcaataagcgaggctggagatgtaacccccgtcttt

SEQ ID NO: 406 - DNA, 53 bp, Artificial
  Synthetic construct
tttgcggagctgattattttggcgcgatgaccctctctacgtaccctctcttt

SEQ ID NO: 407 - DNA, 74 bp, Artificial
  Synthetic construct
tttgcgccaagacctaagtgtgcgtacgacaagaccttgatactcttccagacaagtgtgatgcctccgggttt

SEQ ID NO: 408 - DNA, 61 bp, Artificial
  Synthetic construct
tttttaaaagcttctcactgttatactcgtgtattctgacgcaattgcagtctccagcttt

SEQ ID NO: 409 - DNA, 61 bp, Artificial
  Synthetic construct
tttcatatctggaaccgcggactcagctccgcctattgccgtaaataggggagagggtttt

SEQ ID NO: 410 - DNA, 48 bp, Artificial
  Synthetic construct
tttaccgggttcttgttgtggaggtgtaccacgagtgatgtcccgttt

SEQ ID NO: 411 - DNA, 40 bp, Artificial
  Synthetic construct
ttttgctatgagatacctgcgatctgatgctctgtctttt

SEQ ID NO: 412 - DNA, 40 bp, Artificial
  Synthetic construct
tttccatggaaattgagtagcgtggggtttcatgcatttt

SEQ ID NO: 413 - DNA, 32 bp, Artificial
  Synthetic construct
tttatgcatgaaacccccctgggaacggtttt

SEQ ID NO: 414 - DNA, 48 bp, Artificial
  Synthetic construct
tttattcaattagtctacgttgctcctccacaacaagaacccggtttt

SEQ ID NO: 415 - DNA, 66 bp, Artificial
  Synthetic construct
tttaccatgcagccgagtatctcatagcagccgaggacgggacatcactcaagagcaccacacttt

SEQ ID NO: 416 - DNA, 66 bp, Artificial
  Synthetic construct
tttatgtctcgactggtcaatttccatgggcggccttagacagagcatcaggaacataaattcttt

SEQ ID NO: 417 - DNA, 79 bp, Artificial
  Synthetic construct
tttagatttccgatgttactggatatgccccgctagtacgcgtaaagatcgtcttctggctgtggtcgcctatcctttt

SEQ ID NO: 418 - DNA, 79 bp, Artificial
  Synthetic construct
ttttacgtaccgttacgcgcgagcgccataaatcgactcgtcacaagggagttctggagtggtgaaatgacgctgtttt

SEQ ID NO: 419 - DNA, 66 bp, Artificial
  Synthetic construct
tttataatggtatctccgtatgactgtgggatcaaccgaaaaacacagcgctggggcagtagattt

SEQ ID NO: 420 - DNA, 40 bp, Artificial
  Synthetic construct
tttggttgatcccacagggtggtaagggttctactgcttt

SEQ ID NO: 421 - DNA, 48 bp, Artificial
  Synthetic construct
tttaaaccatcacatacccgtctcacatcggaaatctaagttgtcttt

SEQ ID NO: 422 - DNA, 74 bp, Artificial
  Synthetic construct
tttttgaagtcgggatgtaacggtacgtaggaacgcaactagcggggcattaatggcatgccaacagccagttt

SEQ ID NO: 423 - DNA, 74 bp, Artificial
  Synthetic construct
tttggtaggcattaaagagataccattattccactgtgtcgatttatggctcgaccctccattaccactccttt

SEQ ID NO: 424 - DNA, 79 bp, Artificial
  Synthetic construct
tttgagttacattctcttgtaaactaggccctgtaggggggcgcatcctcaattcctcggggcaggcttatatccgttt

SEQ ID NO: 425 - DNA, 79 bp, Artificial
  Synthetic construct
tttgactatacctgtttgttgtcagctggtgtgcatgctgccaccgataggtctctgaccttccttataccggagcttt

SEQ ID NO: 426 - DNA, 66 bp, Artificial
  Synthetic construct
tttgaattccgtgctgagtacttgaaagcaacgacaggagtagattctgtattcaagatgattttt

SEQ ID NO: 427 - DNA, 40 bp, Artificial
  Synthetic construct
tttctgtcgttgctttaactgcttttaaaaatcatctttt

SEQ ID NO: 428 - DNA, 48 bp, Artificial
  Synthetic construct
tttccggcaacttcttgcggttatgagaatgtaactcctcagcacttt

SEQ ID NO: 429 - DNA, 74 bp, Artificial
  Synthetic construct
tttcattcattgtctaaacaggtatagtcatgctttgcctacagggcctatccctaaagtatggccccgagttt

SEQ ID NO: 430 - DNA, 74 bp, Artificial
  Synthetic construct
ttttctggacgaaggccagcacggaattcacggtactcatgcacaccagcgctgctgccacacgaaggtcattt

SEQ ID NO: 431 - DNA, 79 bp, Artificial
  Synthetic construct
tttcgccaccccttccaacgtcgtgttcttagacgccttttctcgcagtgtgtcctatgaatgtttccgaggcgcgttt

SEQ ID NO: 432 - DNA, 79 bp, Artificial
  Synthetic construct
ttttacgtgggggtatacccgctcatacgaaggttggtggccagcatccccgacacatatctgtgaaacaagtaggttt

SEQ ID NO: 433 - DNA, 66 bp, Artificial
  Synthetic construct
ttttttcaacggaagacaggaaggaattctagagggcattgtgtgcgtaactcgcgtgcacccttt

SEQ ID NO: 434 - DNA, 40 bp, Artificial
  Synthetic construct
tttgccctctagaattgcgaacaattgccgggtgcacttt

SEQ ID NO: 435 - DNA, 48 bp, Artificial
  Synthetic construct
tttcgttcgggtggttgaacttatggaaggggtggcgcgtggggcttt

SEQ ID NO: 436 - DNA, 74 bp, Artificial
  Synthetic construct
tttttcgagagtgtgcatacccccacgtaaccctgttggcgtctaagaactggtcaggttcttcattcatattt

SEQ ID NO: 437 - DNA, 74 bp, Artificial
  Synthetic construct
tttgcaccccgcaccttcttccgttgaaaacttgaaaccaaccttcgtatactggagaacaatcagatatgttt

SEQ ID NO: 438 - DNA, 45 bp, Artificial
  Synthetic construct
tttaacgatactacaataggtcttggcgcgaaccaagagaatttt

SEQ ID NO: 439 - DNA, 45 bp, Artificial
  Synthetic construct
tttaaggaaggcggcagagaagcttttaaagcacagaaatagttt

SEQ ID NO: 440 - DNA, 32 bp, Artificial
  Synthetic construct
tttggaaaagtcattcggttccagatatgttt